(19) United States
(12) Patent Application Publication
(10) Pub. No.: US 2008/0260738 A1
(43) Pub. Date: Oct. 23, 2008

(54) SINGLE CHAIN FC, METHODS OF MAKING AND METHODS OF TREATMENT

(76) Inventors: Margaret D. Moore, Seattle, WA (US); Marshall D. Snavely, Redmond, WA (US); Brian A. Fox, Seattle, WA (US); Gabriela H. Hoyos, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/106,081

(22) Filed: Apr. 18, 2008

Related U.S. Application Data
(60) Provisional application No. 60/912,647, filed on Apr. 18, 2007.
(60) Provisional application No. 60/914,682, filed on Apr. 27, 2007.

(51) Int. Cl. A61K 39/395; C07K 16/18; C12N 15/11; C12N 15/00; A61P 43/00; C12N 1/14; C12N 5/06; C12P 21/04

(52) U.S. Cl. 424/134.1; 530/387.1; 530/387.3; 530/391.1; 536/23.53; 435/320.1; 435/255.1; 435/326; 435/69.6; 424/130.1; 424/138.1

(57) ABSTRACT

The present invention relates generally to scFc molecules. The scFc molecules comprise at least two Fc regions and at least one linker, and can be produced in a variety of single chain configurations. The scFc molecules can further comprise at least one binding entity and/or at least one functional molecule. Binding entities can be fused to the scFc molecule in a variety of configurations. The present invention also relates generally to methods for making such molecules and methods for their use. The scFc molecules provided herein can be recombinantly produced. Also provided are monovalent forms of the scFc molecules that have an equivalent or superior ADCC and/or CDC response than do bivalent molecules targeting the same antigens. Provided herein are improved antigen binding compositions. Methods for using the scFc molecules of the present inventions are provided

### CROSS REFERENCE TO RELATED APPLICATIONS

This application claims the benefit of U.S. Provisional Application Ser. No.60/912,647, filed Apr. 18, 2007 and U.S. Provisional Application Ser. No. 60/914,682 filed Apr. 27, 2008, both of which are incorporated herein by reference.

### FIELD OF THE INVENTION

The present invention relates generally to methods of making and using single chain Fc molecules. These molecules can also comprise binding entities, payload molecules, and entities to improve stability, solubility and half life.

### BACKGROUND OF THE INVENTION

The Fc portion of an antibody molecule includes the CH2 and CH3 domains of the heavy chain and a portion of the hinge region. It was originally defined by digestion of an IgG molecule with papain. Fc is responsible for two of the highly desirable properties of an IgG: recruitment of effector function and a long serum half life. The ability to kill target cells to which an antibody is attached stems from the activation of immune effector pathway (ADCC) and the complement pathway (CDC) through binding of Fc to Fc receptors and the complement protein, C1q, respectively. The binding is mediated by residues located primarily in the lower hinge region and upper CH2 domain (Wines, et al., J. Immunol. (2000) 164, 5313; Woof and Burton, Nature Reviews (2004) 4, 1.). The long half life in serum demonstrated by IgG is mediated through a pH dependant interaction between amino acids in the CH2 and CH3 domains and the neonatal receptor, FcRn (Ghetie and Ward, Immunology Today (1997) 18, 592; Petkova, et al., Int. Immunol. (2006) 18, 1759).

Formation of a dimer, comprising two CH2-CH3 units, is required for the functions provided by intact Fc. Interchain disulfide bonds between cysteines in the hinge region help hold the two chains of the Fc molecule together to create a functional unit. However, even in the absence of the hinge region, the CH3 domains have a strong tendency to associate, leading to the formation of non-covalent dimers (Theis, et al. J. Mol. Biol. (1999) 293, 67; Chames and Baty, FEMS Micorobiol. Lett. (2000) 189, 1). The association between CH3 domains is random and largely independent of other structural domains to which they are attached. The random pairing of CH3 domains limits the types of binding entities that can be attached to the Fc and, unless the units attached to CH2-CH3 are identical, the product formed in a cell is a mixture of homodimers and heterodimers that are very difficult to separate biochemically.

Very few approaches have been developed to direct the pairing of Fc domains and retain effector function while avoiding random association. One method that can be applied to the production of non-random Fc pairing is disclosed in U.S. Pat. No. 5,731,168, which describes methods of preparing heteromultimeric polypeptides such as bispecific antibodies, bispecific immunoadhesins and antibody-immunoadhesin chimeras. This Patent teaches methods that involve introducing a protuberance at the interface of a first polypeptide and a corresponding cavity in the interface of a second polypeptide, such that the protuberance can be positioned in the cavity so as to promote heteromultimer formation and hinder homomultimer formation. “Protuberances” are constructed by replacing small amino acid side chains from the interface of the first polypeptide with larger side chains (e.g. tyrosine or tryptophan). Compensatory “cavities” of identical or similar size to the protuberances are created in the interface of the second polypeptide by replacing large amino acid side chains with smaller ones (e.g. alanine or threonine). The protuberance and cavity can be made by synthetic means such as altering the nucleic acid encoding the polypeptides or by peptide synthesis. None of the multispecific polypeptides produced by the above-described method retain effector function.

Another approach to pairing Fc molecules in a non-random manner was described by Ridgway et al. (“‘Knobs-into-holes’ Engineering of Antibody Ch3 Domains for Heavy Chain Heterodimerization” Protein Engineering 9(7):617-621 (1996)). This approach is based on compensating alterations of two specific, amino acid residues in the CH3 domain that direct formation of heterodimers and prevent formation of homodimers. While this approach can work very well under certain conditions, it has not proven to be generally useful. The formation of the heterodimer is never 100% due to the formation of stable half molecules and the incorrect pairing of heavy and light chains. In order to optimize production, either the light chains must be engineered as well or a pair of antibodies must be selected that share the same light chain. Both of these alternatives are technically demanding and can result in antibodies of lower affinity. See also, Carter, P. 2001. Bispecific human IgG by design. J. Immunol. Meth. 248: 7-15).

While the above described methods may work under certain sets of conditions, none of the methods have proven to be efficient methods of generating Fc molecules capable of forming multispecific, multivalent binding molecules, such as multivalent antibodies and antigen binding molecules, such as antibody fragments. Thus, there remains a need in the art for multispecific, multivalent binding molecules that retain effector function and can be developed into potent therapeutics, while being effectively and efficiently produced at large-scale in any number of available production systems.

### SUMMARY OF THE INVENTION

In one embodiment of the current invention there is provided an scFc polypeptide comprising at least two Fc monomers and a linker. The Fc monomers are joined by said linker to form a single polypeptide. Linkers are known in the art. Some preferred linkers include, but are not limited to: SEQ ID NO:52, SEQ ID NO:53, SEQ ID NO:54, SEQ ID NO:55, SEQ ID NO:56, SEQ ID NO:11 and SEQ ID NO:33. The linker joins the Fc monomers in a variety of configurations, for example, in such configurations as are illustrated in FIG. 1. The scFc polypeptide overcomes the problems in the art associated with dimerization of separate Fc monomers.

Fc monomers of the scFc polypeptide comprise amino acid sequences that are substantially identical to the amino acid sequences of Fc monomers known in the art. By way of example only, and not limitation, the amino acid sequence of an Fc monomer in the scFc polypeptide is preferably at least 80% identical, more preferably at least 85% identical, more preferably at least 90% identical, more preferably at least 95% identical and more preferably 100% identical to the amino acid sequence of an Fc. monomer selected from IgG1 Fc region, an IgG2 Fc region, an IgG3 Fc region, an IgG4 Fc region, an IgM Fc region, an IgA Fc region, an IgD Fc region, an IgE Fc region, Fc1, Fc4, Fc5, Fc6, Fc7, Fc8, Fc9, Fc1O, SEQ ID NO:2; SEQ ID NO:4; SEQ ID NO:22; and SEQ ID NO:31 and variants thereof. It is preferred that the Fc monomers of an scFc polypeptide each have an amino acid sequence that is at least 80% identical, more preferably at least 85% identical, more preferably at least 90% identical, more preferably at least 95% identical and more preferably 100% identical to the amino acid sequence of the respective Fc monomers making up an Fc molecule selected from IgG1 Fc monomers, an IgG2 Fc monomers, an IgG3 Fc monomers, an IgG4 Fc monomers, an IgM Fc monomers, an IgA Fc monomers, an IgD Fc monomers, an IgE Fc monomers, Fc1 monomers, Fc4 monomers, Fc5 monomers, Fc6 monomers, Fc7 monomers, Fc8 monomers, Fc9 monomers, Fc1O monomers, SEQ ID NO:2; SEQ ID NO:4; SEQ ID NO:22; and SEQ ID NO:31 and variants thereof. Fc monomers typically comprise two constant heavy regions, however, some comprise three constant heavy regions and Fc monomer variants may comprise one constant heavy region or fragments of constant heavy regions. Fc monomers may further comprise hinge regions. In one aspect of the current embodiment said scFc polypeptide comprises a first Fc monomer comprising a CH2 domain and a CH3 domain and a second Fc monomer comprising a CH2 domain and a CH3 domain. In a non-limiting example, said first Fc monomer and said second Fc monomer are arranged in an amino to carboxyl order selected from: a) Hinge-CH2-CH3-linker-Hinge-CH2-CH3; b) Hinge-CH2-CH3-linker-CH2-CH3; c) Hinge-CH2-linker-Hinge-CH2-CH3-linker-CH3; d) Hinge-CH2-linker-CH2-CH3-linker-CH3; e) linker-CH2-CH3-linker-CH2-CH3; and f) CH2-linker-CH2-CH3-linker-CH3.

In a further aspect of the preferred embodiment the scFc polypeptides further comprises one or more binding entities. Said binding entities can be fused to the scFc molecule using any technique known in the art. Preferably, the binding entities are fused to said scFc polypeptide using a linker, more preferably a polypeptide linker. In a preferred embodiment of this aspect, said binding entity is a scFv; a Fab; a tascFv, a biscFv, a diabody; a triabody; a single-domain antibody; and a recombinant antibody fragment. Alternatively, the binding entity is a soluble receptor or a ligand-binding fragment thereof. In a further aspect, an scFc polypeptide further comprises at least one functional molecule selected from: a therapeutic agent, a molecule that increases solubility, a molecule that improves stability, and a molecule that extends the half life of said scFc polypeptide, such as PEG.

In one non-limiting example, an scFc polypeptide comprising one or more binding is arranged in an amino to carboxyl order selected from: a) Fc monomer-binding entity-Fc monomer-binding entity; b) binding entity-Fc monomer-Fc monomer-binding entity; c) binding entity-binding entity-Fc monomer-Fc monomer; d) Fc monomer-Fc monomer-binding entity-binding entity; e)Fc monomer- binding entity- Fc monomer; b) binding entity-Fc monomer-Fc monomer; and c) Fc monomer-Fc monomer-binding entity. The Fc monomers and binding entities of these example scFc polypeptides are preferably linked using a linker, more preferably a polypeptide linker.

In one particular aspect, there is provided an scFc polypeptide, wherein each of said scFc polypeptide's two Fc monomers have a polypeptide sequence that is at least 90% identical to an Fc monomer polypeptide sequence of an Fc molecule selected from the group consisting of: SEQ ID NO:2; SEQ ID NO:4; SEQ ID NO:6; SEQ ID NO:8; SEQ ID NO:10; SEQ ID NO:22; SEQ ID NO:31; SEQ ID NO:57 and SEQ ID NO:58. Preferably, said scFc polypeptide further comprises at least one binding entity. More preferably, said polypeptide further comprises one binding entity that binds an antigen selected from: a PDGFR.beta. antigen, a VEGF-A antigen, a HER2/c-erb-2 antigen, an IL-17A antigen, and an IL-23 antigen, a CLA antigen. One such scFc polypeptide comprises a polypeptide sequence at least 95% identical to a sequence selected from: SEQ ID NO:4; SEQ ID NO:22; and SEQ ID NO:31, and a binding entity that is at least 90% identical to a polypeptide sequence selected from the group consisting of SEQ ID NO:44 and SEQ ID NO:38. Another such scFc polypeptide comprises a polypeptide sequence identical to a sequence selected from: SEQ ID NO:4; SEQ ID NO:22; and SEQ ID NO:31, and a binding entity that is at least 90% identical to a polypeptide sequence selected from the group consisting of SEQ ID NO:44 and SEQ ID NO:38. Another such scFc polypeptide comprises a polypeptide sequence identical to a sequence selected from: SEQ ID NO:4; SEQ ID NO:22; and SEQ ID NO:31, and a binding entity that is at least 95% identical to a polypeptide sequence selected from the group consisting of SEQ ID NO:44 and SEQ ID NO:38. Another such scFc polypeptide comprises a polypeptide sequence is at least 95% identical to a sequence selected from: SEQ ID NO:4; SEQ ID NO:22; and SEQ ID NO:31, and a binding entity that is at least 95% identical to a polypeptide sequence selected from the group consisting of SEQ ID NO:44 and SEQ ID NO:38. Another such scFc polypeptide comprises a polypeptide sequence at least 95% identical to a sequence selected from: SEQ ID NO:4; SEQ ID NO:22; and SEQ ID NO:31, and a binding entity that is identical to a polypeptide sequence selected from the group consisting of SEQ ID NO:44 and SEQ ID NO:38. Another such scFc polypeptide is SEQ ID NO:48. Another such scFc polypeptide is SEQ ID NO:64. Another such scFc polypeptide is SEQ ID NO:66. Another such scFc polypeptide is SEQ ID NO:68. Another such scFc polypeptide is at least 95% identical to SEQ ID NO:38. Another such scFc polypeptide is at least 95% identical to SEQ ID NO:48. Another such scFc polypeptide is at least 95% identical to SEQ ID NO:64. Another such scFc polypeptide is at least 95% identical to SEQ ID NO:66. Another such scFc polypeptide is at least 95% identical to SEQ ID NO:68. Another such scFc polypeptide is at least 90% identical to SEQ ID NO:38. Another such scFc polypeptide is at least 90% identical to SEQ ID NO:48. Another such scFc polypeptide is at least 90% identical to SEQ ID NO:64. Another such scFc polypeptide is at least 90% identical to SEQ ID NO:66. Another such scFc polypeptide is at least 90% identical to SEQ ID NO:68. Another such scFc polypeptide is at least 85% identical to SEQ ID NO:38. Another such scFc polypeptide is at least 85% identical to SEQ ID NO:48. Another such scFc polypeptide is at least 85% identical to SEQ ID NO:64. Another such scFc polypeptide is at least 85% identical to SEQ ID NO:66. Another such scFc polypeptide is at least 85% identical to SEQ ID NO:68.

In an alternative aspect, said d scFc polypeptide further comprises two binding entities. Preferably, said scFc polypeptide comprises said two binding entities and is configured as a tascFv-scFc; BiscFv-scFc; or bispecific-scFc. Each of said binding entities can target the same antigen or separate antigens. One such scFc polypeptide comprises two binding entities wherein a first binding entity binds a PDGFR.beta. antigen and a second binding entity binds a VEGF-A antigen. Another such scFc polypeptide comprises two binding entities wherein a first binding entity binds an IL-17A antigen and a second binding entity binds an IL-23 antigen.

In another embodiment there is provided a polynucleotide that encode an scFc molecule comprising at least two Fc monomers and a linker. Said polynucleotide molecules may further encode an scFc molecule comprising one or more binding entities or one or more functional molecules.

In one aspect, said polynucleotide is an element of an expression vector. In this aspect, said polynucleotide is preferably operably linked to additional elements comprising: a transcription promoter; and a transcription terminator. Other elements of expression vectors are known to those skilled in the art. One such expression vector comprises a polynucleotide that encodes in a single open reading frame a polypeptide with at least 90% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58. Another such expression vector comprises a polynucleotide that encodes in a single open reading frame a polypeptide with at least 95% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58. Another such expression vector comprises a polynucleotide that encodes in a single open reading frame a polypeptide with 100% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58.

In one aspect, the polynucleotide encodes an scFc molecule comprising at least two Fc monomers, a linker and one or more binding entities. One such polynucleotide is an element of an expression vector and is preferably operably linked to additional elements comprising: a transcription promoter; and a transcription terminator.

In this aspect, a binding entity of the one or more binding entities preferably binds an antigen selected from the group consisting of: PDGFR.beta., VEGF-A, HER2/c-erb-2, IL-17A, IL-23, and CLA. When the polynucleotide is encoding more than one binding entity, each binding entity may bind the same or separate antigens. One such polynucleotide is an element of an expression vector comprises a binding entity that is at least 85% identical to a polynucleotide sequence selected from the group consisting of SEQ ID NO:37 and SEQ ID NO:43. Another such polynucleotide is an element of an expression vector comprises a binding entity that is at least 90% identical to a polynucleotide sequence selected from the group consisting of SEQ ID NO:37 and SEQ ID NO:43. Another such polynucleotide is an element of an expression vector comprises a binding entity that is at least 95% identical to a polynucleotide sequence selected from the group consisting of SEQ ID NO:37 and SEQ ID NO:43. Another such polynucleotide is an element of an expression vector comprises a binding entity that is 100% identical to a polynucleotide sequence selected from the group consisting of SEQ ID NO:37 and SEQ ID NO:43. Another such polynucleotide is at least 85% identical to SEQ ID NO:47. Another such polynucleotide is at least 85% identical to SEQ ID NO:63. Another such polynucleotide is at least 85% identical to SEQ ID NO:65. Another such polynucleotide is at least 85% identical to SEQ ID NO:67. Another such polynucleotide is at least 90% identical to SEQ ID NO:47. Another such polynucleotide is at least 90% identical to SEQ ID NO:63. Another such polynucleotide is at least 90% identical to SEQ ID NO:65. Another such polynucleotide is at least 90% identical to SEQ ID NO:67. Another such polynucleotide is at least 95% identical to SEQ ID NO:47. Another such polynucleotide is at least 95% identical to SEQ ID NO:63. Another such polynucleotide is at least 95% identical to SEQ ID NO:65. Another such polynucleotide is at least 95% identical to SEQ ID NO:67. Another such polynucleotide is 100% identical to SEQ ID NO:47. Another such polynucleotide is 100% identical to SEQ ID NO:63. Another such polynucleotide is 100% identical to SEQ ID NO:65. Another such polynucleotide is 100% identical to SEQ ID NO:67.

In a further embodiment, there is provided a cultured cell, wherein said cultured cell comprises an exogenous polynucleotide encoding an scFc polypeptide. The scFc polypeptide comprises at least two Fc monomers and a linker. Said polynucleotide molecules may further encode an scFc molecule comprising one or more binding entities or one or more functional molecules.

In one aspect, said polynucleotide is an element of an expression vector. Thus, said cultured cell comprises an expression vector encoding an scFc molecule of the current invention. In this aspect, said polynucleotide is preferably operably linked to additional elements comprising: a transcription promoter; and a transcription terminator. Other elements of expression vectors are known to those skilled in the art. One such expression vector comprises a polynucleotide that encodes in a single open reading frame a polypeptide with at least 90% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58. Another such expression vector comprises a polynucleotide that encodes in a single open reading frame a polypeptide with at least 95% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58. Another such expression vector comprises a polynucleotide that encodes in a single open reading frame a polypeptide with 100% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58.

In one aspect, the polynucleotide encodes an scFc molecule comprising at least two Fc monomers, a linker and one or more binding entities. One such polynucleotide is an element of an expression vector and is preferably operably linked to additional elements comprising: a transcription promoter; and a transcription terminator.

In this aspect, a binding entity of the one or more binding entities preferably binds an antigen selected from the group consisting of: PDGFR.beta., VEGF-A, HER2/c-erb-2, IL-17A, IL-23, and CLA. When the polynucleotide is encoding more than one binding entity, each binding entity may bind the same or separate antigens. One such polynucleotide is an element of an expression vector comprises a binding entity that is at least 85% identical to a polynucleotide sequence selected from the group consisting of SEQ ID NO:37 and SEQ ID NO:43. Another such polynucleotide is an element of an expression vector comprises a binding entity that is at least 90% identical to a polynucleotide sequence selected from the group consisting of SEQ ID NO:37 and SEQ ID NO:43. Another such polynucleotide is an element of an expression vector comprises a binding entity that is at least 95% identical to a polynucleotide sequence selected from the group consisting of SEQ ID NO:37 and SEQ ID NO:43. Another such polynucleotide is an element of an expression vector comprises a binding entity that is 100% identical to a polynucleotide sequence selected from the group consisting of SEQ ID NO:37 and SEQ ID NO:43. Another such polynucleotide is at least 85% identical to SEQ ID NO:47. Another such polynucleotide is at least 85% identical to SEQ ID NO:63. Another such polynucleotide is at least 85% identical to SEQ ID NO:65. Another such polynucleotide is at least 85% identical to SEQ ID NO:67. Another such polynucleotide is at least 90% identical to SEQ ID NO:47. Another such polynucleotide is at least 90% identical to SEQ ID NO:63. Another such polynucleotide is at least 90% identical to SEQ ID NO:65. Another such polynucleotide is at least 90% identical to SEQ ID NO:67. Another such polynucleotide is at least 95% identical to SEQ ID NO:47. Another such polynucleotide is at least 95% identical to SEQ ID NO:63. Another such polynucleotide is at least 95% identical to SEQ ID NO:65. Another such polynucleotide is at least 95% identical to SEQ ID NO:67. Another such polynucleotide is 100% identical to SEQ ID NO:47. Another such polynucleotide is 100% identical to SEQ ID NO:63. Another such polynucleotide is 100% identical to SEQ ID NO:65. Another such polynucleotide is 100% identical to SEQ ID NO:67.

In a further aspect of this embodiment, said cultured cell expresses a sialyltransferase gene. This expressed sialyltransferase gene can be either endogenous or exogenous to said cultured cell. Thus, said cultured cell expresses a sialylated scFc polypeptide of the current invention. One such cell is a yeast cell that is engineered to express a sialyltransferase gene. Another such cell is a mammalian cell that is engineered to express a sialyltransferase gene. Another such cell is a Chinese Hamster Ovary cell that is engineered to express an alpha-2,6-sialyltransferase gene.

In another embodiment, there is provided a method of producing an scFc polypeptide comprising: culturing a cell under conditions wherein an scFc polynucleotide is expressed from an scFc expression vector; and recovering said expressed scFc. Preferably, the cell used in said method comprises an exogenous polynucleotide encoding an scFc polypeptide, wherein said scFc polypeptide comprises at least two Fc monomers and a linker. Said polynucleotide molecule may further encode an scFc molecule comprising one or more binding entities or one or more functional molecules.

In one aspect of this method for producing an scFc polypeptide, the cell comprises a polynucleotide that is an element of an expression vector and said polynucleotide is operably linked to additional elements comprising: a transcription promoter; and a transcription terminator. Other elements of expression vectors are known to those skilled in the art. One such expression vector comprises a polynucleotide that encodes in a single open reading frame a polypeptide with at least 90% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58. Another such expression vector comprises a polynucleotide that encodes in a single open reading frame a polypeptide with at least 95% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58. Another such expression vector comprises a polynucleotide that encodes in a single open reading frame a polypeptide with 100% sequence identity to a polypeptide selected from the group consisting of: SEQ ID NO:2; SEQ ID NO:4; SEQ ID NO:6; SEQ ID NO:8; SEQ ID NO:10; SEQ ID NO:22; SEQ ID NO:31, SEQ ID NO:57 and SEQ ID NO:58.

In one aspect, the polynucleotide encodes an scFc molecule comprising at least two Fc monomers, a linker and one or more binding entities. One such polynucleotide is an element of an expression vector and is preferably operably linked to additional elements comprising: a transcription promoter; and a transcription terminator.

In this aspect, a binding entity of the one or more binding entities preferably binds an antigen selected from the group consisting of: PDGFR.beta., VEGF-A, HER2/c-erb-2, IL-17A, IL-23, and CLA. When the polynucleotide is encoding more than one binding entity, each binding entity may bind the same or separate antigens. One such polynucleotide is an element of an expression vector comprises a binding entity that is at least 85% identical to a polynucleotide sequence selected from the group consisting of SEQ ID NO:37 and SEQ ID NO:43. Another such polynucleotide is an element of an expression vector comprises a binding entity that is at least 90% identical to a polynucleotide sequence selected from the group consisting of SEQ ID NO:37 and SEQ ID NO:43. Another such polynucleotide is an element of an expression vector comprises a binding entity that is at least 95% identical to a polynucleotide sequence selected from the group consisting of SEQ ID NO:37 and SEQ ID NO:43. Another such polynucleotide is an element of an expression vector comprises a binding entity that is 100% identical to a polynucleotide sequence selected from the group consisting of SEQ ID NO:37 and SEQ ID NO:43. Another such polynucleotide is at least 85% identical to SEQ ID NO:47. Another such polynucleotide is at least 85% identical to SEQ ID NO:63. Another such polynucleotide is at least 85% identical to SEQ ID NO:65. Another such polynucleotide is at least 85% identical to SEQ ID NO:67. Another such polynucleotide is at least 90% identical to SEQ ID NO:47. Another such polynucleotide is at least 90% identical to SEQ ID NO:63. Another such polynucleotide is at least 90% identical to SEQ ID NO:65. Another such polynucleotide is at least 90% identical to SEQ ID NO:67. Another such polynucleotide is at least 95% identical to SEQ ID NO:47. Another such polynucleotide is at least 95% identical to SEQ ID NO:63. Another such polynucleotide is at least 95% identical to SEQ ID NO:65. Another such polynucleotide is at least 95% identical to SEQ ID NO:67. Another such polynucleotide is 100% identical to SEQ ID NO:47. Another such polynucleotide is 100% identical to SEQ ID NO:63. Another such polynucleotide is 100% identical to SEQ ID NO:65. Another such polynucleotide is 100% identical to SEQ ID NO:67.

In a further aspect of this embodiment, the cell further expresses a sialyltransferase gene, thus the method provides for producing sialylated scFc polypeptides. This expressed sialyltransferase gene can be either endogenous or exogenous to said cell. One such cell is a yeast cell that is engineered to express a sialyltransferase gene. Another such cell is a mammalian cell that is engineered to express a sialyltransferase gene. Another such cell is a Chinese Hamster Ovary cell that is engineered to express an alpha-2,6-sialyltransferase gene.

In another aspect of the current invention there are provided methods of making a medicament for treating a disorder, pharmaceutical composition and methods of treating disorders.

In one aspect there is a method for treating an immune system disorder in a mammal suspected of suffering from such a disorder comprising administering to said mammal an scFc polypeptide described herein. One such scFc polypeptide comprises two Fc monomers, wherein said two Fc monomers have polypeptide sequences with at least 90% identity to the respective Fc monomer sequences in the group consisting of:; SEQ ID NO:4; SEQ ID NO:6; SEQ ID NO:8; SEQ ID NO:10; SEQ ID NO:22; SEQ ID NO:31 SEQ ID NO:57 and SEQ ID NO:58. Optionally, said scFc polypeptide comprises at least one binding entity, wherein a binding entity binds an antigen selected from the group consisting of: IL-17A, IL-23, and CLA.

In another aspect there is a method for treating a cancer in a mammal suspected of suffering from such a disorder comprising administering to said mammal an scFc polypeptide described herein. One such scFc polypeptide comprises two Fc monomers, wherein said two Fc monomers have polypeptide sequences with at least 90% identity to the respective Fc monomer sequences in the group consisting of:; SEQ ID NO:4; SEQ ID NO:6; SEQ ID NO:8; SEQ ID NO:10; SEQ ID NO:22; SEQ ID NO:31 SEQ ID NO:57 and SEQ ID NO:58. Optionally, said scFc polypeptide comprises at least one binding entity, wherein a binding entity binds an antigen selected from the group consisting of: PDGFR.beta., VEGF-A, and HER2/c-erb-2. One such scFc molecule comprises a binding entity that is about 95% identical to a polypeptide sequence selected from the group consisting of SEQ ID NO:44 and SEQ ID NO:38. Another such scFc molecule comprises a binding entity that is a polypeptide sequence selected from the group consisting of SEQ ID NO:44 and SEQ ID NO:38. Another such scFc molecule comprises a polypeptide sequence that is about 85% identical to SEQ ID NO:48; SEQ ID NO:64; SEQ ID NO:66 or SEQ ID NO:68. SEQ ID NO:38. Another such scFc molecule comprises a polypeptide sequence that is about 90% identical to SEQ ID NO:48; SEQ ID NO:64; SEQ ID NO:66 or SEQ ID NO:68. SEQ ID NO:38. Another such scFc molecule comprises a polypeptide sequence that is about 95% identical to SEQ ID NO:48; SEQ ID NO:64; SEQ ID NO:66 or SEQ ID NO:68. SEQ ID NO:38. Another such scFc molecule comprises a polypeptide sequence that is 100% identical to SEQ ID NO:48; SEQ ID NO:64; SEQ ID NO:66 or SEQ ID NO:68. SEQ ID NO:38.

In a further embodiment there is provided an scFc polypeptide and methods of its use to stimulate NK cells, to stimulate CDC, or to stimulate ADCC. Surprisingly, in many of these methods using an scFc polypeptide comprising a single binding entity (e.g., monovalent) the response is equal to or better than the response received from a bivalent molecule (e.g., a mAb or an Fc fusion molecule). Without being bound to any theory, this surprising result may be due to one or more of: not causing a dimerization of cell surface receptor antigens, which then leads to an internalization of the dimerized receptor and antibody, a 1:1 (scFc:antigen) equimolar ratio of scFc molecules to cell surface target antigens compared to a 1:2 (bivalent:antigen) molar ratio of bivalent molecules to cell surface target antigens; or a more flexible scFc structure compared to a bivalent structure, thereby providing the scFc molecule with a greater range of flexibility for making contact with a complement molecule and/or an Fc receptor of an NK cell. Other possibilities exist. At any rate, there is provided a method of stimulating NK cells in a mammal comprising admixing an scFc polypeptide with cells or tissues of said mammal. Such admixing can take place in vitro (ex vivo) or in vivo, via the administration of an scFc molecule to said mammal. There is also provided a method of stimulating CDC in a mammal comprising admixing an scFc polypeptide with breast cancer cells. Such admixing can take place in vitro (ex vivo) or in vivo, via the administration of an scFc molecule to said mammal. There is also provided a method of stimulating ADCC in a mammal comprising admixing an scFc polypeptide of claim 1 with breast cancer cells. Such admixing can take place in vitro (ex vivo) or in vivo, via the administration of an scFc molecule to said mammal. Preferably, the NK cell stimulation, CDC stimulation and/or ADCC stimulation will lyse a cancer cell. Preferably, a breast cancer cell. Alternatively, the NK cell stimulation, CDC stimulation and/or ADCC stimulation will lyse a cell involved in an immune system disorder. Thus, there is provided a method for treating a disorder, preferably a cancer or an immune system disorder, by administering an scFc molecule of the current invention to stimulate NK cells, CDC, ADCC or a combination thereof. More preferably, when a monovalent scFc molecule is administered such stimulation response is equal to or better than the response generated by a bivalent composition. One such scFc molecule comprises a binding entity that is about 95% identical to a polypeptide sequence selected from the group consisting of SEQ ID NO:44 and SEQ ID NO:38. Another such scFc molecule comprises a binding entity that is a polypeptide sequence selected from the group consisting of SEQ ID NO:44 and SEQ ID NO:38. Another such scFc molecule comprises a polypeptide sequence that is about 85% identical to SEQ ID NO:48; SEQ ID NO:64; SEQ ID NO:66 or SEQ ID NO:68. Another such scFc molecule comprises a polypeptide sequence that is about 90% identical to SEQ ID NO:48; SEQ ID NO:64; SEQ ID NO:66 or SEQ ID NO:68. Another such scFc molecule comprises a polypeptide sequence that is about 95% identical to SEQ ID NO:48; SEQ ID NO:64; SEQ ID NO:66 or SEQ ID NO:68. Another such scFc molecule comprises a polypeptide sequence that is 100% identical to SEQ ID NO:48; SEQ ID NO:64; SEQ ID NO:66 or SEQ ID NO:68.

In a further aspect there is a method for generating an improved pharmaceutical composition relative to a bivalent pharmaceutical composition, wherein said method comprises generating a monovalent scFc molecule targeting the same antigen as said bivalent molecule targets. One such improved pharmaceutical composition comprises a polypeptide sequence that is about 85% identical to SEQ ID NO:48; SEQ ID NO:64; SEQ ID NO:66 or SEQ ID NO:68. Another such improved pharmaceutical composition comprises a polypeptide sequence that is about 90% identical to SEQ ID NO:48; SEQ ID NO:64; SEQ ID NO:66 or SEQ ID NO:68. Another such improved pharmaceutical composition comprises a polypeptide sequence that is about 95% identical to SEQ ID NO:48; SEQ ID NO:64; SEQ ID NO:66 or SEQ ID NO:68. Another such improved pharmaceutical composition comprises a polypeptide sequence that is 100% identical to SEQ ID NO:48; SEQ ID NO:64; SEQ ID NO:66 or SEQ ID NO:68.

### BRIEF DESCRIPTION OF THE DRAWINGS

FIG. 1 (a)-(e) shows diagrammatic representations of the single chain Fc portion of the binding molecules of the present invention, with the hinge represented by light lines, the Gly-Ser linkers in heavy lines, inter-domain disulfide bonds in dashed lines, CD8 stalk in heavy beaded line, CH2 domains in striped ovals, and CH3 domains in gray ovals; (a) scFc10.1; (b) scFc10.2; (c) scFc10.3; (d) scFc10.4; (e) scFc10.5.

FIG. 2 shows the comparison of the wild type human .gamma.1 constant region Fc (also referred to as “Fc1”) with Fc4, Fc5, Fc6, Fc7, Fc8, Fc9, and Fc10. The human wild type .gamma. I constant region sequence was first described by Leroy Hood's group in Ellison et al., Nucl. Acids Res. 10:4071 (1982). EU Index positions 356, 358, and 431 define the G1m .gamma.1 haplotype. The wild type sequence shown here is of the G1m(1), positions 356 and 358, and nG1m(2), position 431, haplotype. The CH1 domain of the human .gamma.1 constant region is not part of the Fc and is therefore not shown. The locations of the hinge region, the CH2 domain, and the CH3 domain are indicated. The Cys residues normally involved in disulfide bonding to the light chain constant region (LC) and heavy chain constant region (HC) are indicated. A “.” indicates identity to wild type at that position. Only locations where the Fc variants differ from wild type are shown, otherwise the Fc sequences match the wild type sequence shown. The sequence positions are numbered according to the universally accepted EU Index numbering system for immunoglobulin proteins. * * * indicates the location of the carboxyl terminus and is included to clarify the difference in the carboxyl terminus of Fc6 relative to the other Fc versions.

FIGS. 3 (a) and (b) shows annotated cDNA sequence (and corresponding amino acid sequence) of the scFc10.1 intermediate construct (SEQ ID NOs: 1 and 2) and final scFc10.1 construct (SEQ ID NOs:3 and 4). The heavy chain constant regions are denoted as CH2 and CH3.

FIGS. 4 (a) and (b) shows annotated cDNA sequence (and corresponding amino acid sequence) of the scFc10.2 intermediate construct (SEQ ID NOs:19 and 20) and final scFc10.2 construct (SEQ ID NOs:21 and 22). The heavy chain constant regions are denoted as CH2 and CH3.

FIGS. 5 (a) and (b) shows annotated cDNA sequence (and corresponding amino acid sequence) of the scFc10.3 intermediate construct (SEQ ID NOs: 28 and 29) and final scFc10.3 construct (SEQ ID NOs: 30 and 3 1). The heavy chain constant regions are denoted as CH2 and CH3.

FIG. 6 (a)-(c) shows that the addition of single chain Fc molecules ((a) is scFc10.1, (b) is scFc10.2, and (c) is scFc10.3) does not block immune complex precipitation in an anti-OVA/OVA immune complex precipitation assay based upon MOller NPH (1979) Immunology 38: 631-640 and Gavin AL et al., (1995) Clin Exp Immunol 102: 620-625.

FIG. 7 shows the results from an assay measuring IL-6 and TNF.alpha. accumulation from MC/9 cells incubated with anti-OVA/OVA immune complexes in the presence of increasing amounts of scFc10.1, scFc10.2 and scFc10.3. The results show that scFc10.1 was most potent at blocking immune complex-mediated cytokine secretion, scFc10.3 was slightly less potent and scFc10.2 showed little or no inhibition of IL-6 and TNF.alpha. secretion.

FIG. 8 shows that human NK cells stimulated with human IL-21 in combination with scFc10.1, scFc10.2, or scFc10.3 produced 2-3 times more IFN-.gamma. than NK cells stimulated with IL-21 alone.

FIG. 9 depicts some possible scFc fusion points.

FIG. 10 (a)-(b) are CDC assays comparing cytolysis by herceptin; herceptin scFv-scFc; herceptin scFv-Fc10; human Fc10; and scFc alone, when the complement source is freshly thawed human serum (a), or freeze thawed human serum (b).

FIG. 11 (a)-(b) are ADCC assays comparing cytolysis by control; anti-PDGFR.beta. monoclonal antibody; Fc10 with PDGFR.beta.-binding scFv; and scFc10.1 with PDGFR.beta.-binding scFv, when the NK cells were grown in human serum (a), or FBS (b).

FIG. 12 plots data received from a Western Blot assay and illustrates that SEQ ID NO:48, SEQ ID NO:60 and Herceptin similarly bind to FcRn at pH6.0, indicating that the monovalent scFc molecules retain antibody binding properties significant for enhanced half-life in vivo when compared to these bivalent molecules.

### DETAILED DESCRIPTION OF THE INVENTION

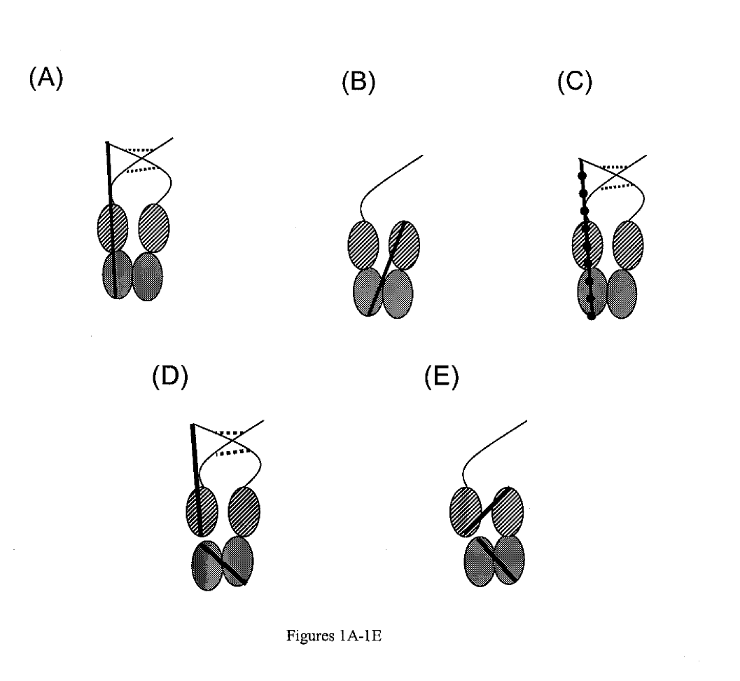

The present invention is directed a single expression construct for combining at least two Fc monomers to form a single chain Fc molecule (scFc). In one embodiment, scFc monomers are combined using a linker. Exemplary configurations for combining Fc monomers to form an scFc molecule are shown in FIG. 1. Other configurations within the scope of this currently disclosed invention will be constructed by the ordinarily skilled artisan. In another embodiment, the present invention further comprises an scFc molecule combined with one or more binding entities. In one aspect, the binding entities are combined to the scFc molecule using a linker. An scFc molecule can be combined with at least two binding entities to form multispecific, multivalent binding molecules. In a further embodiment there are provided methods of making the scFc molecules of the invention and methods for using the same.

The scFc molecules of the present invention are based on the discovery of methods that allow the formation of a functional Fc dimer from a single polypeptide unit, thereby avoiding the existing problem in the art of random association of CH3 subunits. Preferably, these molecules of the present invention comprise an Fc fragment of an antibody and a binding entity that can target or specifically bind to a desired target antigen (e.g., target polypepfide). Any binding entity or combination of binding entities can be covalently attached to the single chain Fc polypeptide to combine binding specificity, with antibody-like effector function, and/or long serum half life in a single molecule, resulting in a binding molecule within the scope of the present invention.

These and other features of this invention will now be described with reference to the drawings and preferred embodiments as described above, the definitions and examples described below, all of which are intended to illustrate and not to limit the invention.

Definitions. In the description that follows, a number of terms are used extensively. The following definitions are provided to facilitate understanding of the invention.

The terms “a,” “an,” and “the” include plural referents, unless the context clearly indicates otherwise.

As used herein, the term “antigen” or “target antigen” encompasses any substance or material that is specifically recognized by a binding entity, such as an antibody or antibody fragment or multispecific binding molecule of the present invention. Preferably, an antigen is a target polypeptide as defined herein.

A “target polypeptide” or a “target peptide” is an amino acid sequence that comprises at least one epitope, and that is a preferred antigen for the binding of the binding molecules of the present invention. A target polypeptide may be expressed on a target cell, such as a tumor cell, or a cell that carries an infectious agent antigen or it may e a soluble polypeptide such a ligand. T cells recognize peptide epitopes presented by a major histocompatibility complex molecule to a target polypeptide or target peptide and typically lyse the target cell or recruit other immune cells to the site of the target cell, thereby killing the target cell. A “target gene” is the polynucleotide sequence that encodes a “target polypeptide.”

The term “tumor associated antigen” refers to a peptide or polypeptide or peptide complex that has a different expression profile from antigen found on a non-tumor cells. For example, a non-tumor antigen may be expressed in higher frequency or density by tumor cells than it is by non-tumor cells. A tumor antigen may differ from a non-tumor antigen structurally, for example, the antigen could be expressed as a truncated polypeptide, have some mutation in the amino acid sequence or polynucleotide sequence encoding the antigen, be misfolded, or improperly modified post-translationally. Similar to antigens that are present on normal, non-tumor cells in the host organism allow the tumor cells to escape the host's immunological surveillance mechanisms. The term tumor associated antigen, as used herein, refers to a subset of antigen or target antigen.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Thus, as used herein, the term “antibody” or “antibody peptide(s)” refers to an intact antibody, or a fragment thereof that competes with the intact antibody for specific binding and includes chimeric, humanized, fully human, and multispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, F(ab′).sub.2, Fv, and single-chain antibodies. As used herein, the term “immunoglobulin” refers to a protein consisting of one or more polypeptides substantially encoded by immunoglobulin genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. “Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide-linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al., J. Mol. Biol. 186:651 (1985); Novotny and Haber, Proc. Natl. Acad. Sci. U.S.A. 82:4592 (1985)).

Full-length immunoglobulin “light chains” (about 25 Kd or 214 amino acids) are encoded by a variable region gene at the NH.sub.2—terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the constant region gene (about 330 amino acids). Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7).

An immunoglobulin light or heavy chain variable region consists of a “framework” region interrupted by three hypervariable regions. Thus, the term “hypervariable region” refers to the amino acid residues of the variable regions an antibody which bind to an antigen. The hypervariable region comprises amino acid residues from a “Complementarity Determining Region” or “CDR” in the light chain variable domain and in the heavy chain variable domain (Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (LI), 50-52 (L2) and 91-96 (L3) in the light chain variable domain and 26-32 (Hi), 53-55 (H2) and 96-101 (H3) in the heavy chain variable domain; Chothia and Lesk, 1987, J. Mol. Biol. 196: 901-917) (both of which are incorporated herein by reference). “Framework Region” or “FR” residues are those variable domain residues other than the hypervariable region residues as herein defined. The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of an antigen.

As is used herein, the term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor”. Constant regions need not be present, but if they are, they must be substantially identical to human immunoglobulin constant regions, e.g., at least about 85-90%, preferably about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human. One approach, described in EP 0239400 to Winter et al. describes the substitution of one species' complementarity determining regions (CDRs) for those of another species, such as substituting the CDRs from human heavy and light chain immunoglobulin variable region domains with CDRs from mouse variable region domains. These altered antibodies may subsequently be combined with human immunoglobulin constant regions to form antibodies that are human except for the substituted murine CDRs which are specific for the antigen. Methods for grafting CDR regions of antibodies may be found, for example in Riechmann et al. (1988) Nature 332:323-327 and Verhoeyen et al. (1988) Science 239:1534-1536.

In addition to antibodies, immunoglobulins may exist in a variety of other forms including, for example, single-chain or Fv, Fc, and F(ab′)2, Fab, as well as diabodies, linear antibodies, multivalent or multispecific hybrid antibodies (as described above and in detail in: Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988)). (See, generally, Hood et al., “Immunology”, Benjamin, N.Y., 2nd ed. (1984), and Hunkapiller and Hood, Nature, 323, 15-16 (1986)).

The term “isolated antibody” as used herein refers to an antibody that has been identified and separated and/or recovered from a component of its natural environment or from an environment in which it was recombinantly produced. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present.

The term “parent antibody” as used herein refers to an antibody which is encoded by an amino acid sequence used for the preparation of the variant. Preferably, the parent antibody has a human framework region and, if present, has human antibody constant region(s). For example, the parent antibody may be a humanized or human antibody.

As used herein, the term “binding affinity” refers to the strength of the interaction between a single antigen-binding site on a binding molecule of the present invention and its specific antigen epitope. The higher the affinity, the tighter the association between antigen and antibody, and the more likely the antigen is to remain in the binding site. Binding affinity is represented by an affinity constant Ka, which is the ratio between the rate constants for binding and dissociation of antibody and antigen. Typical affinities for IgG antibodies are 105-109 L/mole. Antibody affinity is measured by equilibrium dialysis, which is well-known by those skilled in the art. The relationship between bound and free antigen and antibody affinity is expressed by the Scatchard equation, r/c=Kn−Kr, where r=the ratio of [bound antigen] to [total antibody], c=[free antigen], K=affinity, and n=number of binding sites per antibody molecule (“valence”; defined herein).

“Avidity” is the functional affinity of multiple antigen molecules binding to multivalent binding molecules such as antibodies, or the binding molecules of the present invention. Avidity strengthens binding to antigens with repeating identical epitopes. The more antigen-binding sites an individual antibody molecule has, the higher its avidity for antigen.

The term “agonist” refers to any compound including a protein, polypeptide, peptide, antibody, antibody fragment, large molecule, or small molecule (less than 10 kD), that increases the activity, activation or function of another molecule.

The term “antagonist” refers to any compound including a protein, polypeptide, peptide, antibody, antibody fragment, large molecule, or small molecule (less than 10 kD), that decreases the activity, activation or function of another molecule.

A “binding entity” comprises a polypeptide, polynucleotide or small molecule that is capable of binding another peptide, polypeptide, or polynucleotide. Binding entities encompassed by the present invention include, but are not limited to, peptides, polypeptides, recombinant antibody fragments, such as classic monovalent antibody fragments like Fab and scFv, and engineered antibody fragments like diabodies, triabodies, minibodies and single-domain antibodies. Thus, a binding entity can comprise any molecule that binds to an antigen or extracellularly expressed protein. The binding entities of the invention can additionally be linked to therapeutic payloads, such as radionuclides, toxins, enzymes, liposomes and viruses, as well as payloads that are engineered for enhanced therapeutic efficacy, such as PEG. An scFc polypeptide described herein can be used without a binding entity, or can further comprise one or more binding entities. The scFc polypeptides further comprising one or more binding entities can be monovalent, bivalent, multivalent, monospecific, bispecific or multispecific. Such scFc polypeptides comprising one or more binding arms can be designed so that a binding entity has an selected binding affinity towards an antigen. Targets for bispecific or multispecific molecules generally fall into the following categories: (a) both targets were not previously known to have that indication or use; (b) one target has a known indication or use and the second target was never previously known to have that indication or use; (c) both targets have the same or a similar indication or use, but have never been characterized as being capable of co-binding; (d) one or both targets have a known indication or use, it would be therapeutically efficacious to bind both, but the targets are not candidates for co-binding; or (e) both targets share homology such that a conserved domain can be identified on each Targets and used to generate one antibody that binds both Targets. Biscpecific or multispecific scFc molecules can be designed accordingly. (See, e.g., Handl, et al. Expert Opin. Ther. Targets, 8(6), 565-86 (2004); and Gilles, et al., Expert Opin. Ther. Targets, 7(2), 137-9 (2003)).

A “bivalent molecule” is a molecule that comprises at least two binding entities. A “multivalent molecule” is a molecule that comprises more than two, (such as three, four, five, or more) binding entities

A “bispecific” or “bifunctional” molecule comprises binding entities specificity for two different target antigens or target polypeptides. A “multispecific” molecule is a molecule that comprises more than two, (such as three, four, five, or more) binding entities having antigenic specificity for different antigens or target polypeptides.

As used herein, the term “Fc portion” or “Fc monomer” means a polypeptide comprising at least one CH2 domain and one CH3 domain of an immunoglobulin molecule. An Fc monomer can be a polypeptide comprising at least a fragment of the constant region of an immunoglobulin excluding the first constant region immunoglobulin domain of the heavy chain (CH1), but maintaining at least part of one CH2 domain and one CH3 domain, wherein the CH2 domain is amino terminal to the CH3 domain. In one aspect of this definition, an Fc monomer can be a polypeptide constant region comprising a portion of the hinge region, a CH2 region and a CH3 region. Such Fc polypeptide molecules can be obtained by papain digestion of an immunoglobulin region, for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of: an IgG1 Fc region, an IgG2 Fc region, an IgG3 Fc region, an IgG4 Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two constant region immunoglobulin domains of IgA, IgD, and IgG, and the last three constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between CH1 and CH2. Although the boundaries of the Fc portion may vary, the human IgG heavy chain Fc portion is usually defined to comprise residues C226 or P230 to its carboxyl-terminus, wherein the numbering is according to the EU index as in Kabat. The Fc portion may refer to this region in isolation, or this region in the context of an Fc polypeptide, as described below. By “Fc polypeptide” as used herein is meant a polypeptide that comprises all or part of an Fc monomer. Fc polypeptides include antibodies, Fc fusions, isolated Fc molecules, functional Fc fragments and functional variants thereof.

“Fc fusion” as used herein means a protein wherein one or more polypeptides (including another Fc portion as shown in FIG. 1, or for example a binding entity like a scFv molecule) are operably linked to an Fc portion or a derivative thereof. An Fc fusion combines the Fc portion with a fusion partner, which in general can be any protein or small molecule (including another Fc portion as shown in FIG. 1, or for example a binding entity like a scFv molecule, or both). The effect of the fusion partner may be to mediate target binding (such as, for example, cell proliferation, apoptosis, tissue differentiation, cellular migration) via at least one binding entity, and thus it is functionally analogous to the variable regions of an antibody (e.g., an scFv molecule). Virtually any protein or small molecule may be linked to Fc portion to generate an Fc fusion. Protein fusion partners may include, but are not limited to, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, preferably an extracellularly expressed protein (such as a receptor or cell differentiating protein), that is implicated in disease. Specific examples of particular drugs that may serve as Fc fusion partners can be found in L. S. Goodman et al., Eds., Goodman and Gilman's The Pharmacological Basis of Therapeutics (McGraw-Hill, New York, ed. 9, 1996). A variety of linkers, defined and described below, may be used to covalently link Fc to a fusion partner, such as another Fc to generate an Fc fusion (like the scFc molecules described herein and shown in FIG. 1).

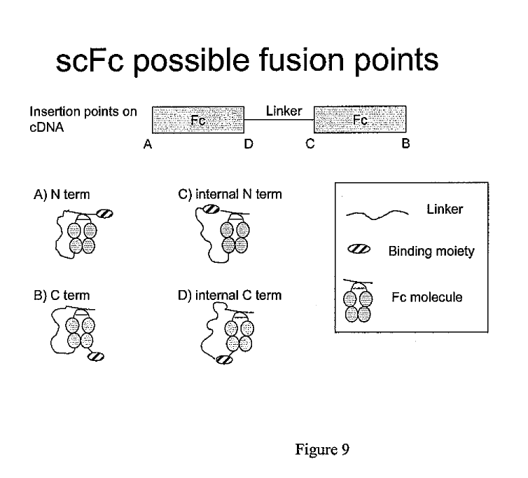

As used herein, the terms “single-chain Fc,” “scFc” “scFc polypeptide” or “scFc molecule” are used interchangeably and refer to a molecule comprising at least two Fc portions within a single polypeptide chain. Non-limiting examples of scFc molecules can be found in FIGS. 1 and 9, herein.

The term “chimeric antibody” or “chimeric antibody fragment” refers to antibodies or fragments thereof, whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3 or the scFc described herein. A typical therapeutic chimeric antibody is thus a hybrid protein composed of the variable or antigen-binding domain from a mouse antibody and the constant domain from a human antibody, although other mammalian species may be used. In this way, the antigen-binding portion of the parent monoclonal antibody is grafted onto the backbone of another species' antibody.

The term “effective neutralizing titer” as used herein refers to the amount of binding molecule or antibody present in the serum of animals (human or cotton rat) that has been shown to be either clinically efficacious (in humans) or to reduce disease symptoms.

As used herein, the term “epitope” refers to the portion of an antigen or target antigen to which a binding entity molecule of the present invention (or antibody or antibody fragment) specifically binds. Thus, the term “epitope” includes any protein determinant capable of specific binding to a binding entity of the invention.

The term “epitope tagged” when used herein refers to a binding molecule of the present invention fused to an “epitope tag”. The epitope tag polypeptide has enough residues to provide an epitope against which an antibody can be made, yet is short enough such that it does not interfere with activity of antibodies of the present invention. The epitope tag preferably is sufficiently unique so that the antibody thereagainst does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 (Field et al. Mol. Cell. Biol. 8:2159-2165 (1988)); the c-myc tag and the 8F9, 3C7, 6E10, G4, B7 and 9E10 antibodies thereto (Evan et al., Mol. Cell. Biol. 5(12):3610-3616(1985)); and the Herpes Simplex virus glycoprotein D (gD) tag and its antibody (Paborsky et al., Protein Engineering 3(6):547-553(1990)). In certain embodiments, the epitope tag is a “salvage receptor binding epitope.” As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

The term “fragment” as used herein refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of any of the multispecific antibody or antibody fragment of the present invention.

As used herein, the term “human antibody” includes an antibody that has an amino acid sequence of a human immunoglobulin and includes antibodies isolated from human immunoglobulin libraries or from animals transgenic for one or more human immunoglobulin genes and that do not express endogenous immunoglobulins, as described, for example, by Kucherlapati et al in U.S. Pat. No. 5,939,598.

As used herein, the terms “single-chain Fv,” “single-chain antibodies,” “Fv” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

The term “hybrid,” as used herein, means that sequences encoding two or more Fc domains of different origin are present in the Fc portion of the binding molecules of the present invention. In the present invention, various types of hybrids are possible. That is, domain hybrids may be composed of one to four domains selected from the group consisting of CH1, CH2, CH3 and CH4 of IgG1 Fc, IgG2 Fc, IgG3 Fc and IgG4 Fc, and may include the hinge region. On the other hand, IgG is divided into IgG1, IgG2, IgG3 and IgG4 subclasses, and the present invention includes combinations and hybrids thereof.

As used herein, the term “deglycosylation” means that sugar moieties are enzymatically removed from a binding entity of the invention. The term “aglycosylation” means that a binding entity is produced in an unglycosylated form by a prokaryote, preferably E. coli.

A “F(ab′)2 fragment” contains two light chains and two heavy chains containing a portion of the constant region between the CH1 and CH2 domains, such that an interchain disulfide bond is formed between two heavy chains.

The term “diabodies” refers to small antibody-like fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The term “linear antibodies” refers to the antibodies described in Zapata et al. Protein Eng. 8(10):1057-1062 (1995). Briefly, these antibodies comprise a pair of tandem Fd segments (VH-CH1-VH-CH1) which form a pair of antigen binding regions. Linear antibodies can be bispecific or monospecific.

The term “immunologically functional immunoglobulin fragment” or the term “immunologically functional antibody fragment” may be used interchangeably and as used herein refers to a polypeptide fragment that contains at least the variable domains of the immunoglobulin heavy and light chains. An immunologically functional antibody fragment of the invention is capable of binding to a ligand or receptor, or any desired target antigen, and initiating a desired response, whether that be preventing binding of a ligand to its receptor, interrupting the biological response resulting from ligand binding to receptor, or any combination thereof.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The terms “nucleic acid” or “nucleic acid molecule” refer to a deoxyribonucleotide or ribonucleotide polymer in either single-or double-stranded form, and unless otherwise limited, would encompass known analogs of natural nucleotides that can function in a similar manner as naturally occurring nucleotides. A “nucleotide sequence” also refers to a polynucleotide molecule or oligonucleotide molecule in the form of a separate fragment or as a component of a larger nucleic acid. The nucleotide sequence or molecule may also be referred to as a “probe” or a “primer.” Some of the nucleic acid molecules of the invention are derived from DNA or RNA isolated at least once in substantially pure form and in a quantity or concentration enabling identification, manipulation, and recovery of its component nucleotide sequence by standard biochemical methods. Examples of such methods, including methods for PCR protocols that may be used herein, are disclosed in Sambrook et at., Molecular Cloning: A Laboratory Manual, 2nd ed., Cold Spring Harbor Laboratory Press, New York (1989), Ausubel, F. A., et al., eds., Current Protocols in Molecular Biology, John Wiley and Sons, Inc., New York (1987), and Innis, M., et al., (Eds.) PCR Protocols: A Guide to Methods and Applications, Academic Press, San Diego, Calif. (1990). Reference to a nucleic acid molecule also includes its complement as determined by the standard Watson-Crick base-pairing rules, with uracil (U) in RNA replacing thymine (T) in DNA, unless the complement is specifically excluded. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like.

As described herein, the nucleic acid molecules of the invention include DNA in both single-stranded and double-stranded form, as well as the DNA or RNA complement thereof. DNA includes, for example, DNA, genomic DNA, chemically synthesized DNA, DNA amplified by PCR, and combinations thereof. Genomic DNA, including translated, non-translated and control regions, may be isolated by conventional techniques, e.g., using any one of the cDNAs of the invention, or suitable fragments thereof, as a probe, to identify a piece of genomic DNA which can then be cloned using methods commonly known in the art.

A “nucleic acid molecule construct” is a nucleic acid molecule, either single-stranded or double-stranded, that has been modified through human intervention to contain segments of nucleic acid combined and juxtaposed in an arrangement not existing in nature.

As used herein a “nucleotide probe” or “probe” is defined as an oligonucleotide or polynucleotide capable of binding to a target nucleic acid of complementary sequence through one or more types of chemical bonds, through complementary base pairing, or through hydrogen bond formation. Probes are typically used for identification of target molecules.

As used herein an “oligonucleotide primer pair,” “oligonucleotide primer pair member,” “oligonucleotide primer member,” “oligonucleotide primer,” “primer member” or “primer” is defined as an oligonucleotide or polynucleotide capable of binding to a target nucleic acid of complementary sequence through one or more types of chemical bonds, through complementary base pairing, or through hydrogen bond formation. Primers are typically used for amplification of target molecules. It is understood that when discussing oligonucleotide primer pair members in reference to a sequence the primer members are complementary to either the sense or antisense strand, depending on whether the primer member is a 5′ (forward) oligonucleotide primer member, or a 3′ (reverse) oligonucleotide primer member, respectively. The polynucleotide sequences of oligonucleotide primer members disclosed herein are shown with their sequences reading 5′-3′ and thus the 3′ primer member is the reverse complement of the actual sequence. Ordinarily skilled artisans in possession of this disclosure will readily design 5′ and 3′ primer members capable of engineering thrombin cleavage sites into pre-pro-activator molecules.

A “target nucleic acid” herein refers to a nucleic acid to which a nucleotide primer or probe can hybridize. Probes are designed to determine the presence or absence of the target nucleic acid, and the amount of target nucleic acid. Primers are designed to amplify target nucleic acid sequences. The target nucleic acid has a sequence that is significantly complementary to the nucleic acid sequence of the corresponding probe or primer directed to the target so that the probe or primer and the target nucleic acid can hybridize. Preferably, the hybridization conditions are such that hybridization of the probe or primer is specific for the target nucleic acid. As recognized by one of skill in the art, the probe or primer may also contain additional nucleic acids or other moieties, such as labels, which may not specifically hybridize to the target. The term target nucleic acid may refer to the specific nucleotide sequence of a larger nucleic acid to which the probe is directed or to the overall sequence (e.g., gene or mRNA). One skilled in the art will recognize the full utility under various conditions.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces a multispecific antibody or antibody fragment of the present invention from an expression vector.

A “fusion protein” or a “fusion polypeptide” is a hybrid protein or polypeptide expressed by a nucleic acid molecule comprising nucleotide sequences of at least two genes of portions thereof. For example, a fusion protein can comprise at least part of a Fc domain fused with a second polypeptide with a desired property, such as antigen binding or that binds an affinity matrix.

The term “receptor” denotes a cell-associated protein that binds to a bioactive molecule termed a “ligand.” The effect of the ligand on the cell is mediated through this interaction. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

As used herein, the term “isolated,” in reference to polynucleotides, polypeptides or proteins, means that the polynucleotide, polypeptide or protein is substantially removed from polynucleotides, polypeptides, proteins or other macromolecules with which it, or its analogues, occurs in nature. Although the term “isolated” is not intended to require a specific degree of purity, typically, the protein will be at least about 75% pure, more preferably at least about 80% pure, more preferably at least about 85% pure, more preferably at least about 90% pure, more preferably still at least about 95% pure, and most preferably at least about 99% pure.

A polypeptide “variant” as referred to herein means a polypeptide substantially homologous to a native polypeptide, but which has an amino acid sequence different from that encoded by any of the nucleic acid sequences of the invention because of one or more deletions, insertions or substitutions. Amino acid sequence insertions include amino- and/or carboxyl-terminal fusions ranging in length from one residue to polypeptides containing a hundred or more residues, as well as intrasequence insertions of single or multiple amino acid residues. Intrasequence insertions (e.g., insertions within the target polypeptide sequence) may range generally from about 1 to 10 residues, more preferably 1 to 5, most preferably 1 to 3. Variants can comprise conservatively substituted sequences, meaning that a given amino acid residue is replaced by a residue having similar physiochemical characteristics. See, Zubay, Biochemistry, Addison-Wesley Pub. Co., (1983). It is a well-established principle of protein and peptide chemistry that certain amino acids substitutions, entitled “conservative” amino acid substitutions, can frequently be made in a protein or a peptide without altering either the confirmation or the function of the protein or peptide. Such changes include substituting any of isoleucine (I), valine (V), and leucine (L) for any other of these amino acids; aspartic acid (D) for glutamic acid (E) and vice versa; glutamine (Q) for asparagine (N) and vice versa; and serine (S) for threonine (T) and vice versa. Ordinarily, variants will have an amino acid sequence having at least 75% amino acid sequence identity with the reference sequence, more preferably at least 80%, more preferably at least 85%, more preferably at least 90%, and most preferably at least 95%. Identity or homology with respect to this sequence is defined herein as the percentage of amino acid residues in the candidate sequence that are identical with the reference sequence residues, after aligning the sequences and introducing gaps, if necessary, to achieve the maximum percent sequence identity. Preferably, variants will retain the primary function of the parent from which it they are derived.

The above-mentioned substitutions are not the only amino acid substitutions that can be considered “conservative.” Other substitutions can also be considered conservative, depending on the environment of the particular amino acid. For example, glycine (G) and alanine (A) can frequently be interchangeable, as can be alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments. The effects of such substitutions can be calculated using substitution score matrices such PAM120, PAM-200, and PAM-250 as discussed in Altschul, (J. Mol. Biol. 219:55565 (1991)). Other such conservative substitutions, for example, substitutions of entire regions having similar hydrophobicity characteristics, are well known.

Naturally-occurring peptide variants are also encompassed by the invention. Examples of such variants are proteins that result from alternate mRNA splicing events or from proteolytic cleavage of the polypeptides described herein. Variations attributable to proteolysis include, for example, differences in the N- or C-termini upon expression in different types of host cells, due to proteolytic removal of one or more terminal amino acids from the polypeptides encoded by the sequences of the invention.

A “variant” antibody, including “variant” antibody fragments, refers herein to a molecule which differs in amino acid sequence from a “parent” antibody amino acid sequence by virtue of addition, deletion and/or substitution of one or more amino acid residue(s) in the parent antibody sequence. In the preferred embodiment, the variant comprises one or more amino acid substitution(s) in one or more hypervariable region(s) of the parent antibody. For example, the variant may comprise at least one, e.g. from about one to about ten, and preferably from about two to about five, substitutions in one or more hypervariable regions of the parent antibody. A variant antibody or antibody fragment retains the ability to bind to the desired target and preferably has properties which are superior to those of the parent antibody. For example, the variant may have a stronger binding affinity. A variant antibody of particular interest herein is one which displays at least about 10 fold, preferably at least about 20 fold, and most preferably at least about 50 fold, enhancement in biological activity when compared to the parent antibody. The sites of greatest interest for substitutional mutagenesis include the CDRs, FR and hinge regions. They include substitutions of cysteine for other residue and insertions which are substantially different in terms of side-chain bulk, charge, end/or hydrophobicity.

Variants of the scFc molecules of the invention may be used to attain desired characteristics relative such as for example; enhancement or reduction in activity, (e.g., receptor and/or complement binding affinities). A variant or site direct mutant may be made by any methods known in the art. Variants and derivatives of native polypeptides can be obtained by isolating naturally-occurring variants, or the nucleotide sequence of variants, of other or species, or by artificially programming mutations of nucleotide sequences coding for native activators. These variants may include, inter alia: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. The scFc molecules of the present invention may include variants in which amino acid residues from one species are substituted for the corresponding residue in another species, either at the conserved or nonconserved positions. In another embodiment, amino acid residues at nonconserved positions are substituted with conservative or nonconservative residues. The techniques for obtaining these variants, including genetic (suppressions, deletions, mutations, etc.), chemical, and enzymatic techniques, are known to the person having ordinary skill in the art. The present invention also includes fragments, such as antibody fragments like Fc. A “fragment” refers to polypeptide sequences which are preferably at least about 40, more preferably at least to about 50, more preferably at least about 60, more preferably at least about 70, more preferably at least about 80, more preferably at least about 90, and more preferably at least about 100 amino acids in length, and which retain some biological activity or immunological activity (e.g., effector function).

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of mRNA into one or more polypeptides.

As used herein, the term “immunomodulator” includes cytokines, stem cell growth factors, lymphotoxins, co-stimulatory molecules, hematopoietic factors, and the like, and synthetic analogs of these molecules.

As used herein, a “therapeutic agent” is a molecule or atom which is conjugated to a scFc molecule to produce a conjugate which is useful for therapy. Examples of therapeutic agents include drugs, toxins, immunomodulators, chelators, boron compounds, photoactive agents or dyes, and radioisotopes.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide residue as the corresponding position in the second sequence, then the molecules are identical at that position. The percent identity between the two sequences is a function of the number of identical positions shared by the sequences (e.g., % identity=# of identical positions/total # of positions (e.g., overlapping positions) x 100). In certain embodiments, the two sequences are the same length.

The phrase “substantially identical” means that a relevant sequence is at least 70%, 75%, 80%, 85%, 90%, 92%, 95% 96%, 97%, 98%, or 99% identical to a given sequence. By way of example, such sequences may be allelic variants, sequences derived from various species, or they may be derived from the given sequence by truncation, deletion, substitution or addition or amino acid or nucleotide residues. Percent identity between two sequences is determined by standard alignment algorithms such as ClustalX when the two sequences are in best alignment according to the alignment algorithm.

“Similarity” or “percent similarity” in the context of two or more polypeptide sequences, refer to two or more sequences or subsequences that have a specified percentage of amino acid residues that are the same or conservatively substituted when compared and aligned for maximum correspondence. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art. These terms are also applicable to two or more polynucleotide sequences.

The term “substantial similarity,” in the context of polypeptide sequences, indicates that a polypeptide region has a sequence with at least 70% or at least 75%, typically at least 80% or at least 85%, and more typically at least 85%, at least 90%, or at least 95% sequence similarity to a reference sequence. For example, a polypeptide is substantially similar to a second polypeptide, for example, where the two peptides differ by one or more conservative substitutions.

Numerical ranges recited for purity, similarity, identity and fold activity are inclusive of all whole (e.g., 70%, 75%, 79%, 87%, 93%, 98%) and partial numbers (e.g., 72.15, 87.27%, 92.83%, 98.11%) embraced within the recited range numbers, therefore forming a part of this description. For example, an amino acid sequence with 200 residues that share 85% identity with a reference sequence would have 170 identical residues and 30 non-identical residues. Similarly, for example, a polynucleotide sequence with 235 nucleotides may have 200 nucleotide residues that are identical to a reference sequence, thus the polynucleotide sequence will be 85.11% identical to the reference sequence. The terms “at least 80%” and “at least 90%” are also inclusive of all whole or partial numbers within the recited range. For example, at least about 80% pure means that an isolated polypeptide is isolated from other polypeptides, polynucleotides, proteins and macromolecules to a purity of between 80% and 100%, said range being all inclusive of the whole and partial numbers. Thus, 82.5% pure and 91% pure both fall within this purity range. As is used herein, the terms “greater than 95% identical” or “greater than 95% identity” means that an amino acid sequence, for example, shares 95.01%-100% sequence identity with a reference sequence. This range is all inclusive as described immediately above. Those ordinarily skilled in the art will readily calculate percent purity, percent similarity and percent identity.

The determination of percent identity or percent similarity between two sequences can be accomplished using a mathematical algorithm. A preferred, non-limiting example of a mathematical algorithm utilized for the comparison of two sequences is the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches can be performed with the NBLAST program, score=100, wordlength=12 to obtain nucleotide sequences homologous to a nucleic acid encoding a protein of interest. BLAST protein searches can be performed with the XBLAST program, score=50, wordlength=3 to obtain amino acid sequences homologous to protein of interest. To obtain gapped alignments for comparison purposes, Gapped BLAST can be utilized as described in Altschul et al., (Nucleic Acids Res. 25:3389-3402, 1997). Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. (See, e.g., the National Center for Biotechnology Information (NCBI) website, www.ncbi.nlm.nih.gov.) Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci. 10:3-5, 1994); and FASTA described in Pearson and Lipman (Proc. Natl. Acad. Sci. USA 85:2444-8, 1988). Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see, e.g., bioweb.pasteur.fr/docs/man/man/fasta.1.html#sect2, the contents of which are incorporated herein by reference.

Alternatively, protein sequence alignment may be carried out using the CLUSTAL W algorithm, as described by Higgins et al., (Methods Enzymol. 266:383-402, 1996).

Due to the imprecision of standard analytical methods, molecular weights and lengths of polymers are understood to be approximate values. When such a value is expressed as “about” X or “approximately” X, the stated value of X will be understood to be accurate to ±10%.

As is used herein, the term “cancer,” the term “cancer cell” and the term “neoplasm” is used to refer to a diverse group of diseases characterized by uncontrolled division of cells and the ability of these cells to invade other tissues, either by direct growth into adjacent tissue into distant sites by metastasis.

As is used herein, the term “tumor” is used to refer to a swelling or a lump, which can be neoplastic, inflammatory or other. However, it is commonly used when referring to a neoplasm, and can be either benign or malignant.

As is used herein, the term “carcinoma” is used to refer to malignant tumors derived from epithelial cells.

As is used herein, the term “lymphoma” and the term “leukemia” are used to refer to malignant tumors derived from blood or bone marrow.

As is used herein, the term “sarcoma” is used to refer to is used to refer to malignant tumors that begin in bone, cartilage, fat, muscle, blood vessels, or other connective or supportive tissue or mesenchymal cells.

The term “co-administration” is used herein to denote that an scFc composition designed to for administration at a therapy to a particular disorder and another agent may be given concurrently or at different times of a treatment cycle. The co-administration may be a single co-administration of both the scFc composition and the second agent or multiple cycles of co-administration, where both the scFc composition and the second agent are given, at least once, within a treatment period. Co-administration need not be the only times either the scFc composition or the other agent is administered to a patient and either agent may be administered alone or in a combination with additional therapeutic agents.

The term “combination therapy” is used herein to denote that a subject is administered at least one therapeutically effective dose of an scFc composition and another agent. The scFc composition may be a mature polypeptide, fragment thereof, fusion or conjugate.

The term “level” when referring to immune cells, such as NK cells, T cells, B cells and the like, denotes increased level as either an increased number of cells or enhanced activity of cell function and decreased level as a decreased number of cells or diminished activity of cell function.

The term “optimal immunological dose” is defined as the dose of an scFc composition alone or in combination with another agent, wherein the dose is designed to achieve an optimal immunological response.

The term “optimal immunological response” refers to a change in an immunological response after administration of an scFc composition alone or in combination with another agent over the change in immunological response that seen when a non-scFc therapeutic agent alone is administered. The change in immunological response can be: (1) an increase in the number of activated or tumor specific CD8 T cells; (2) an increase in the number of activated or tumor specific CD8 T cells expressing higher levels of granzyme B or perforin or IFN.gamma.; (3) upregulation of the Fc.gamma. receptor (e.g. CD16, CD32, or CD64) on Nk cells, monocytes, or neutrophils; (4) an increase in soluble CD25 in the serum; (5) reduction in serum level of proteins liberated by tumor cells (See, Taro et al., J. Cell Physiol. 203(1):1-5, 2005), for example, carcinoembryonic antigen (CEA), IgG, CA-19-9, or ovarian cancer antigen (CA125); (6) an increase in the numbers of NK cells expressing higher levels of granzyme B, perforin or IFN.gamma.; (7) increase in the levels of activation cytokines such as IL-18, IL-15, IFN.gamma. and chemokines that enable homing of effector cells to the tumor, such as IP-10, RANTES, IL-8, MIP1a or MIP1b; (8) an increase in the numbers of activated macrophages in the periphery or at the tumor site, where activation can be detected by expression of increased MHC Class I or Class II, production of IL-15, IL-18, IFN.gamma., or IL-21; or (9) macrophage activity as indicated by decline in red blood cell count (severity of anemia). These and other biomarkers for determining immunologic responses are known to those skilled in the art, as are the application of the appropriate biomarker(s) to the specific indication being treated.

The term “progression free survival” (PFS) is used herein to be defined as the time from randomization until objective tumor progression or death. For non-randomized studies, PFS is defined as the time from first dose of study medication until objective tumor progression or death.

The term “synergistic” is used herein to denote a biological or clinical activity of two or more therapeutic agents that when the activity is measured it is greater than the activity of either agent alone.

A “therapeutically effective amount” of a composition is that amount that produces a statistically significant effect, such as a statistically significant reduction in disease progression or a statistically significant improvement in organ function. The exact dose will be determined by the clinician according to accepted standards, taking into account the nature and severity of the condition to be treated, patient traits, etc. Determination of dose is within the level of ordinary skill in the art.

Agents used for treating cancer can act either directly or indirectly or both. Direct anti-tumor action generally means the agent (a) activates a cell death pathway, (b) blocks necessary cancer cell growth factors, or (c) delivers cytotoxic agents to the cancer cells. For example, monoclonal antibodies are considered to act on cancer cells directly. Indirect anti-tumor action by a agent can be (a) blocking a negative immunoregulatory host mechanism, such as inhibiting signaling receptors expressed on T regulatory cells; or (b) enhancing the anti-tumor activity of immune effector cells, such as NK cells, cytotoxic T cells, B cells or antigen presenting cells (APCs).

The present invention is directed to scFc molecules that are capable of being used alone or with a fusion partner and methods of making and using the same. Specifically, the present invention is based on the novel concept of attaching at least one fusion partner (such as a binding entity) to a single chain Fc molecule (scFc) to produce a molecule with the potential to be a multispecific and/or multivalent therapeutic. Surprisingly, it was discovered that the use of such an scFc addressed the need for the accommodation of the structural properties of binding entities such as antibody fragment polypeptide subunits while retaining the desirable functional properties of the Fc portion of an antibody. Even more surprisingly, it was also discovered that such an scFc molecule alone (e.g., without any additional attached binding entities) is in fact a potent therapeutic useful in treating inflammation.

As discussed above, the Fc portion of an antibody comprises the CH2 and CH3 domains of an immunoglobulin molecule. The propensity of the hinge and CH3 domains of an antibody to associate and the proximity associated within a single chain construct make it possible for two Fc portions connected by a polypeptide linker and one or more binding entities to fold properly. Thus the scFc molecule produces a multispecific molecule with Fc functions such as effector function and improved half-life. Ideally, the binding molecules of the present invention are produced as a single polypeptide unit and bind two different targets while retaining the important functions of the Fc moiety.

In one aspect the invention provides an scFc molecule that is a single chain polypeptide comprising a two Fc portions with substantially the same characteristics as a native Fc molecule. In an embodiment, the scFc molecule comprises one or more therapeutic agents. In another embodiment, the scFc molecule comprises one or more binding entities. In another embodiment the scFc molecule comprises one or more other entities that confer improved stability, solublility, and/or half-life. In another embodiment the scFc molecule comprises one or more binding entities and one or more therapeutic agents. In another embodiment the scFc molecule comprises one or more binding entities and one or more other entities that confer improved stability, solublility, and/or half-life. In another embodiment the scFc molecule comprises one therapeutic agent and one or more other entities that confer improved stability, solublility, and/or half-life. In another embodiment, the scFc molecule comprises one or more binding entities, one or more therapeutic agents and more other entities that confer improved stability, solublility, and/or half-life.

Such Fc portions include native amino acid sequence and sequence variants thereof (such as Fc5 (SEQ ID NO:8) and Fc10 (SEQ ID NO:10)). An amino acid sequence variant is a sequence that is different from the native amino acid sequence due to a deletion, an insertion, a non-conservative or conservative substitution or combinations thereof at one or more amino acid residue positions. For example, in an IgG Fc, some of the amino acid residues known to be important in binding are at positions 214 to 238, 297 to 299, 318 to 322, or 327 to 331. One or more of these residues may be used as a suitable target for modification.

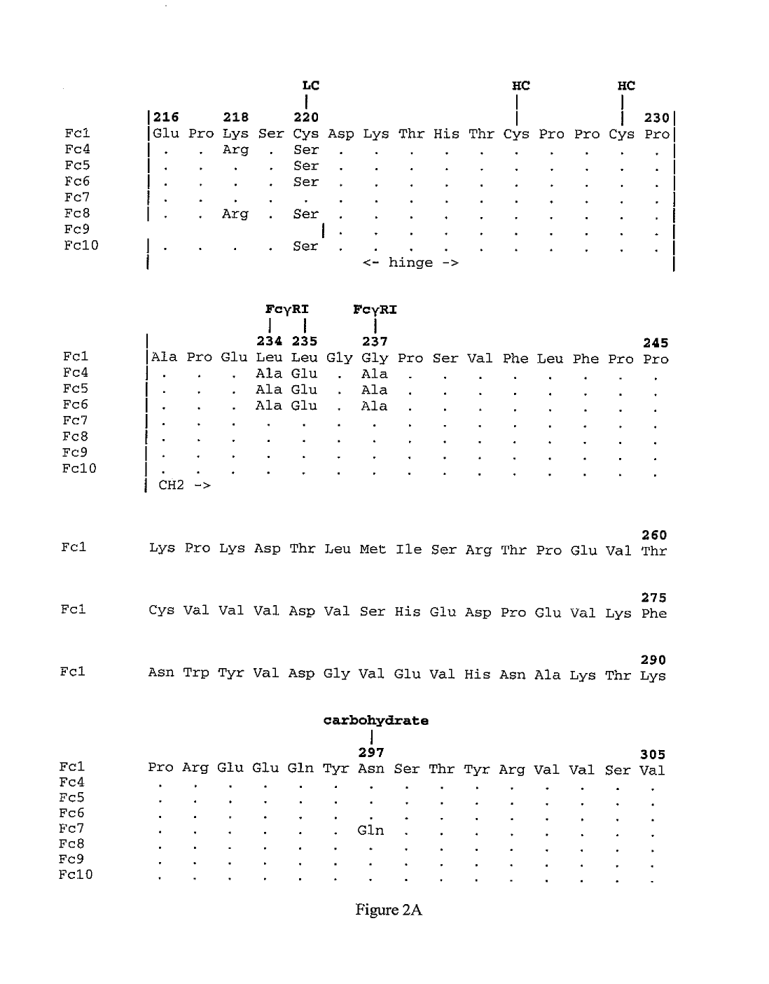

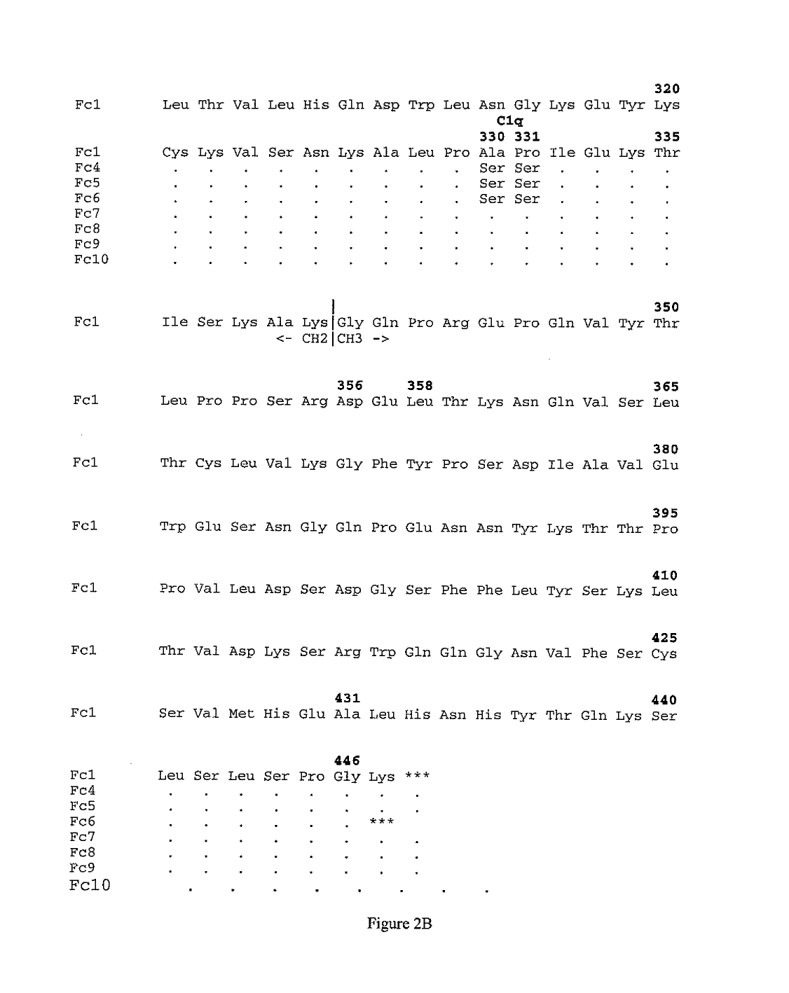

Moreover, the following Fc variants may be used for the Fc portion of the binding molecules of the invention. FIG. 2 shows the comparison of the wild type human .gamma.1 constant region Fc (herein designated as Fc1) with Fc4, Fc5, Fc6, Fc7, Fc8, Fc9, and Fc10, any of which could be used as the Fc portion of the binding molecules of the invention. The human wild type .gamma.1 constant region sequence was first described by Leroy Hood's group in Ellison et al., Nucl. Acids Res. 10:4071 (1982). EU Index positions 356, 358, and 431 define the G1m .gamma.1 haplotype. The wild type sequence shown here is of the G1m(1), positions 356 and 368, and nG1m(2), position 431, haplotype. The other Fc variants are described below in comparison to the Fc1 amino acid sequence.

Fc4 (Effector function minus .gamma.1 Fc with Bg1II site; SEQ ID NOs:5 and 6; FIG. 2). Arg 218 was introduced in the hinge region to include a Bg1II restriction enzyme recognition sequence to facilitate cloning. Cys 220 is the Cys residue that forms the disulfide bond to the light chain constant region in an intact immunoglobulin IgG1 protein. Since the Fc fusion protein constructs do not have a light chain partner, Fc4 includes a Ser for Cys residue substitution to prevent deleterious effects due to the potential presence of an unpaired sulfhydral group. In the CH2 region three amino acid substitutions were introduced to reduce Fc.gamma.receptorI (Fc.gamma.RI) binding. These are the substitutions at EU index positions 234, 235, and 237. These substitutions were described by Greg Winter's group in Duncan et al., Nature 332:563 (1988) and were shown in that paper to reduce binding to the Fc.gamma.RI.

Two amino acid substitutions in the complement C1q binding site were introduced to reduce complement fixation. These are the substitutions at EU index positions 330 and 331. The importance, or relevance, of positions 330 and 331 in complement C1q binding (or lack of complement fixation or activation) is described by Sherie Morrison's group in Tao et al., J. Exp. Med. 178:661 (1993) and Canfield and Morrison, J. Exp. Med. 173:1483 (1991). The CH3 region in the Fc4 variant remains identical to the wild type .gamma.1 Fc.

Fc5 (Effector function minus .gamma.1 Fc without the Bg1II site; SEQ ID NOs:8 and 9; FIG.2) Fc5 is a variant of Fc4. In the Fc5 hinge region the Arg 218 substitution was returned to the wild type Lys 218 residue. Fc5 contains the same Cys 220 to Ser substitution as described above for Fc4. Fc5 contains the same CH2 substitutions as does Fc4, and the Fc5 CH2 region is identical to the wild type .gamma.1 Fc.

Fc6 (Effector function minus .gamma.1 Fc without the Bg1II site and lacking the C-terminal Lys residue; FIG. 2 and SEQ ID NO:57). The Fc6 variant contains the same hinge region substitutions as Fc5 and contains the same CH2 substitutions as Fc4. The Fc6 CH3 region does not contain a carboxyl terminal lysine residue. This particular Lys residue does not have an assigned EU index number. This lysine is removed to a varying degree from mature immunoglobulins and therefore predominantly not found on circulating antibodies. The absence of this residue on recombinant Fc fusion proteins may result in a more homogeneous product.

Fc7 (Aglycosylated .gamma.1 Fc; FIG. 2 and SEQ ID NO:58). The Fc7 variant is identical to the wild type .gamma.1 Fc in the hinge region. In the CH2 region the N-linked carbohydrate attachment site at residue Asn-297 is changed to Gln to produce a deglycosylated Fc. (See e.g., Tao and Morrison (1989) J. Immunol. 143:2595-2601). The CH3 region is identical to the wild type .gamma.1 Fc.

Fc8 variant (.gamma.1 Fc with Cys 220 to Ser substitution and Bg1 II site; FIG. 2) has a hinge region that is identical to Fc4, and both the CH2 region and the CH3 region are identical to the corresponding wild type .gamma.1 Fc regions.

The Fc9 (wild type .gamma.1 Fc with shortened hinge (amino-terminal 5 residues removed); FIG. 2) variant contains a shortened hinge starting at the Asp residue just carboxy-terminal to the Cys residue involved in disulfide linkage to the light chain. The remaining hinge sequence is identical to the wild type. Both the CH2 region sequence and the CH3 region sequence are identical to the corresponding regions for the wild-type .gamma.1 Fc.

The Fc10 variant (wild type .gamma.1 Fc with Cys 220 to Ser substitution; SEQ ID NOs: 9 and 10; FIG.2) contains the same hinge region substitution as Fc5. Both the CH2 region sequence and the CH3 region sequence are identical to the corresponding regions for the wild-type .gamma.1 Fc.

Other Fc variants are possible, including without limitation one in which a region capable of forming a disulfide bond is deleted, or in which certain amino acid residues are eliminated at the N-terminal end of a native Fc form or a methionine residue is added thereto. Thus, in one embodiment of the invention, one or more Fc portions of the scFc molecule can comprise one or more mutations in the hinge region to eliminate disulfide bonding. In yet another embodiment, the hinge region of an Fc can be removed entirely. In still another embodiment, the scFc molecule can comprise an Fc variant.

Further, an Fc variant can be constructed to remove effector functions by substituting, deleting or adding amino acid residues to effect complement binding or Fc receptor binding. For example, and not limitation, a deletion may occur in a complement-binding site, such as a C1q-binding site. Techniques of preparing such sequence derivatives of the immunoglobulin Fc fragment are disclosed in International Patent Publication Nos. WO 97/34631 and WO 96/32478. In addition, the Fc domain may be modified by phosphorylation, sulfation, acrylation, glycosylation, methylation, farnesylation, acetylation, amidation, and the like.

Fc variants can have a biological activity that is either modified or is identical or substantially similar to the native Fc biological activity, depending on the intended use of an scFc molecule. For example, variants may have amino acid deletions, additions or substitutions that confer characteristics such as have improved structural stability, for example, against heat, pH, or the like, or a desired biological activity.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g. Pichia Pastoris, and mammalians cell line, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al., J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al., BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the function of Immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See e.g., Kaneko, et al., Science 313:760 (2006)), whereas removal of fucose from the IgG leads to enhanced ADCC activity (See e.g., Shoji-Hosaka, et al., J. Biochem., 140:777 (2006)).

The CH2 and CH3 domains may be derived from humans or other animals including cows, goats, swine, mice, rabbits, hamsters, rats and guinea pigs, and preferably humans, or synthetic, or a combination thereof. In addition, the Fc portion may be derived from IgG, IgA, IgD, IgE and IgM, or that is made by combinations thereof or hybrids thereof. More specifically, the scFc molecules of the present invention are based on the joining of two Fc portions by a linker to form a multimer.

The linkers can be naturally-occurring, synthetic or a combination of both. For example, a synthetic linker can be a randomized linker, e.g., both in sequence and size. In one aspect, the randomized linker can comprise a fully randomized sequence, or optionally, the randomized linker can be based on natural linker sequences. The linker can comprise, e.g, a non-polypeptide moiety, a polynucleotide, a polypeptide or the like. A linker can be rigid, or alternatively, flexible, or a combination of both. Linker flexibility can be a function of the composition of both the linker and the subunits that the linker interacts with. A suitable length is, e.g., a length of at least one and typically fewer than about 50 amino acid residues, such as 2-25 amino acid residues, 5-20 amino acid residues, 5-15 amino acid residues, 8-12 amino acid residues or 11 residues. Other suitable polypeptide linker sizes may include, e.g., from about 2 to about 15 amino acids, from about 3 to about 15, from about 4 to about 12, about 10, about 8, or about 6 amino acids. The amino acid residues selected for inclusion in the linker polypeptide should exhibit properties that do not interfere significantly with the activity or function of the polypeptide multimer. Thus, the peptide linker should, on the whole, not exhibit a charge that would be inconsistent with the activity or function of the linked polypeptides, or interfere with internal folding, or form bonds or other interactions with amino acid residues in one or more of the domains that would seriously impede the linked polypeptides in question. Preferred linkers include polypeptide linkers such as Gly4Ser as described in Examples 1 and 2. In still another embodiment, the polypeptide linker is a section of the stalk region of human CD8 alpha chain (SEQ ID NO:33).

The linker can also be a non-peptide linker, such as a non-peptide polymer. The term “non-peptide polymer”, as used herein, refers to a biocompatible polymer including two or more repeating units linked to each other by a covalent bond excluding the peptide bond. Examples of the non-peptide polymer include poly (ethylene glycol), poly (propylene glycol), copolymers of ethylene glycol and propylene glycol, polyoxyethylated polyols, polyvinyl alcohol, polysaccharides, dextran, polyvinyl ether, biodegradable polymers such as PLA (poly (lactic acid) and PLGA (poly (lactic-glycolic acid), lipid polymers, chitins, and hyaluronic acid. The most preferred is poly (ethylene glycol) (PEG).

In one embodiment, linkers are used to join two Fc monomers to form an scFc molecule. Sample configurations for linking Fc monomers to form an scFc molecule can be found in FIG. 1. A linker can also be used to join selected binding entities to an scFc molecule, and/or to another binding entity (e.g., two separate polypeptides or proteins, such as two different antibodies). Configurations of molecules comprising an scFc and optionally comprising one or more binding entities are described herein. Linkers to join polypeptide fragments are generally known in the art and can be used to form scFc molecules in accordance of the present invention. Linkers allow the separate, discrete domains to cooperate yet maintain their separate properties. In some cases, a disulfide bridge exists between two linked binding entities or between a linker and a binding entity.

Choosing a suitable linker for an scFc or an scFc comprising one or more binding entities may depend on a variety of parameters including, e.g., the nature of the Fc domains being linked, the nature of any one or more binding entities, the structure and nature of the target to which the composition should bind, and/or the stability of the linker (e.g., peptide linker) towards proteolysis and oxidation.

Particularly suitable linker polypeptides predominantly include amino acid residues selected from Glycine (Gly), Serine (Ser), Alanine (Ala), and Threonine (Thr). For example, the peptide linker may contain at least 75% (calculated on the basis of the total number of residues present in the peptide linker), such as at least 80%, at least 85%, or at least 90% of amino acid residues selected from Gly, Ser, Ala, and Thr. The peptide linker may also consist of Gly, Ser, Ala and/or Thr residues only. The linker polypeptide should have a length that is adequate to link two Fc monomers, and optionally, one or more binding entities to an scFc or to each other in such a way that the linked regions assume the correct conformation relative to one another so that they retain the desired activity.

One example where the use of peptide linkers is widespread is for production of single-chain antibodies where the variable regions of a light chain (VL) and a heavy chain (VH) are joined through an artificial linker, and a large number of publications exist within this particular field. A widely used peptide linker is a 15mer consisting of three repeats of a Gly-Gly-Gly-Gly-Ser amino acid sequence ((Gly4Ser)3) (SEQ ID NO:52). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). Peptide linkers have been used to connect individual chains in hetero- and homo-dimeric proteins such as the T-cell receptor, the lambda Cro repressor, the P22 phage Arc repressor, IL-12, TSH, FSH, IL-5, and interferon-.gamma. Peptide linkers have also been used to create fusion polypeptides. Various linkers have been used, and, in the case of the Arc repressor, phage display has been used to optimize the linker length and composition for increased stability of the single-chain protein (See Robinson and Sauer, Proc. Natl. Acad. Sci. USA 95, 5929-5934, 1998).

Still another way of obtaining a suitable linker is by optimizing a simple linker (e.g., (Gly4Ser)n) through random mutagenesis.

As stated, a linker can be rigid, or flexible, or a combination of both. Linker flexibility can be a function of the composition of both the linker and the binding entities domains that the linker interacts with (e.g., the scFv or Fc domains). The linker joins two Fc monomers, two selected binding entities or an Fc monomer and a selected binding entity and maintains them as separate and discrete entities. Thus the linker can allow the separate discrete Fc monomers and/or binding entities to remain connected in a way that each binding entity binds its respective target(s). In one embodiment, it is generally preferred that the peptide linker possess at least some flexibility. Accordingly, in some variations, the peptide linker contains 1-25 glycine residues, 5-20 glycine residues, 5-15 glycine residues, or 8-12 glycine residues. Particularly suitable peptide linkers typically contain at least 50% glycine residues, such as at least 75% glycine residues. In some embodiments, a peptide linker comprises glycine residues only.

In certain variations, the peptide linker comprises other residues in addition to the glycine. Preferred residues in addition to glycine include Ser, Ala, and Thr, particularly Ser. One example of a specific peptide linker includes a peptide linker having the amino acid sequence Glyx-Xaa-Glyy-Xaa-Glyz (SEQ ID NO:53), wherein each Xaa is independently selected from Alanine (Ala), Valine (Val), Leucine (Leu), Isoleucine (Ile), Methionine (Met), Phenylalanine (Phe), Tryptophan (Trp), Proline (Pro), Glycine (Gly), Serine (Ser), Threonine (Thr), Cysteine (Cys), Tyrosine (Tyr), Asparagine (Asn), Glutamine (Gln), Lysine (Lys), Arginine (Arg), Histidine (His), Aspartate (Asp), and Glutamate (Glu), and wherein x, y, and z are each integers in the range from 1-5. In some embodiments, each Xaa is independently selected from the group consisting of Ser, Ala, and Thr. In a specific variation, each of x, y, and z is equal to 3 (thereby yielding a peptide linker having the amino acid sequence Gly-Gly-Gly-Xaa-Gly-Gly-Gly-Xaa-Gly-Gly-Gly (SEQ ID NO:54), wherein each Xaa is selected as above).

In some cases, it may be desirable or necessary to provide some rigidity into the peptide linker. This may be accomplished by including proline residues in the amino acid sequence of the peptide linker. Thus, in another embodiment, a peptide linker comprises at least one proline residue in the amino acid sequence of the peptide linker. For example, a peptide linker can have an amino acid sequence wherein at least 25% (e.g., at least 50% or at least 75%) of the amino acid residues are proline residues. In one particular embodiment of the invention, the peptide linker comprises proline residues only.

In some embodiments, a peptide linker is modified in such a way that an amino acid residue comprising an attachment group for a non-polypeptide moiety is introduced. Examples of such amino acid residues may be a cysteine or a lysine residue (to which the non-polypeptide moiety is then subsequently attached). Another alternative is to include an amino acid sequence having an in vivo N-glycosylation site (thereby attaching a sugar moiety (in vivo) to the peptide linker). An additional option is to genetically incorporate non-natural amino acids using evolved tRNAs and tRNA synthetases (see, e.g., U.S. Patent Application Publication 2003/0082575) into a polypeptide binding entity or peptide linker. For example, insertion of keto-tyrosine allows for site-specific coupling to an expressed polypeptide.

In certain variations, a peptide linker comprises at least one cysteine residue, such as one cysteine residue. For example, in some embodiments, a peptide linker comprises at least one cysteine residue and amino acid residues selected from the group consisting of Gly, Ser, Ala, and Thr. In some such embodiments, a peptide linker comprises glycine residues and cysteine residues, such as glycine residues and cysteine residues only. Typically, only one cysteine residue will be included per peptide linker. One example of a specific peptide linker comprising a cysteine residue includes a peptide linker having the amino acid sequence Glyn-Cys-Glym (SEQ ID NO:55), wherein n and m are each integers from 1-12, e.g., from 3-9, from 4-8, or from 4-7. In a specific variation, such a peptide linker has the amino acid sequence GGGGG-C-GGGGG (SEQ ID NO:56).

The linkers used to join the Fc monomers of an scFc molecule may be positioned between the CH3 of a first Fc monomer and the CH2 of a second Fc monomer. More specifically, a single chain construct can be designed such that a linker may be placed between any of the following: CH2-CH2, CH2-CH3, CH3-CH3, CH2-CH3 and CH2-CH3, CH2-CH2 and CH3-CH3, CH2-hinge region, CH3-hinge region, CH3 of a first Fc monomer—CH2 of a second Fc monomer, and CH2 of a first Fc monomer—CH3 of a second Fc monomer, as long as the scFc molecule forms the a desired structure. This scFc can then also be combined with one or more binding entities and/or one more therapeutic agents and/or one or more proteins useful to improve stability. The scFc molecule is described with reference to Fc molecules having two constant regions. As described above, scFc molecules can be constructed for the Fc monomers of any immunoglobulin, including, but not limited to IgA, IgD, IgE, IgG and IgM. (See, e.g., Kabat, Structural Concepts in Immunology and Immunochemistry, 2Ed. (Holt 1976)).

Binding entities and therapeutic agents are joined to the scFc molecule by linkers using a variety of techniques known in the art. For example, combinatorial assembly of polynucleotides encoding selected monomer domains can be achieved by restriction digestion and re-ligation, by PCR-based, self-priming overlap reactions, or other recombinant methods. The linker can be attached before the binding entity is identified for its ability to bind to a target or after the binding entity has been selected for the ability to bind to a target.

As described above, the invention comprises a single chain Fc portion linked to at least one binding entity. A binding entity refers to a peptide, polypeptide or any equivalent thereof that has the ability to specifically bind a target antigen or target polypeptide. For example, a binding entity can be an antibody fragment that retains antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like, as well as engineered antibody fragments like diabodies, triabodies, minibodies and single-domain antibodies. The binding entities of the invention can further be linked to therapeutic payloads, such as radionuclides, toxins, enzymes, liposomes and viruses, and engineered for enhanced therapeutic efficacy.

In some embodiments, two or more binding entities are fused to a single chain Fc to form a multivalent binding molecule. In some such embodiments, a multivalent binding molecule comprising a single chain Fc comprises at least two binding entities having binding specificities for different target antigens, thereby generating a multispecific binding molecule. Particularly suitable multispecific binding molecules include multispecific antibodies. In certain variations, a multispecific binding molecule comprising a single chain Fc is a bispecific binding molecule having binding specificity for two different target antigens (e.g., a bispecific antibody).

In an embodiment, the binding entities comprise an Fc portion attached or fused to at least one binding entity, wherein said binding entity is a scFv. ScFvs are recombinant antibody fragments consisting of the variable domains of the heavy and light chains, which are connected by any of the flexible polypeptide linkers described herein. These fragments conserve the binding affinity and the specificity of the parent monoclonal antibody (MAb) and can be efficiently produced in bacteria. (See e.g., WO 05/037989).

The binding entity can be attached to the Fc portion of the scFc as shown in FIG. 9. Specifically, the binding entity can be attached via any of the linkers described herein at any of the following positions: N terminal of the Fc portion; C terminal of the Fc portion; an internal N terminal position within the linker; or a C terminal position within the linker. Thus, in one embodiment, at least one binding entity is fused to the Fc portion of the scFc molecule at the hinge region via any of the linkers described herein. In another embodiment, at least one binding entity is fused to the Fc portion of the scFc molecules of the present invention at the N terminus of the CH2 domain. However, the binding entity (or entities) may be fused to the Fc portion wherever appropriate, as may be determined by one skilled in the art, so as not to limit folding and/or purification of the entire molecule.

Thus, the present invention also comprises scFc molecules linked to at least one binding entity, wherein said binding entity is an scFv specific for a target polypeptide or target antigen. In another embodiment, the scFc molecule of the invention comprises more than one binding entity, wherein said binding entities are scFvs fused to the Fc portion via a linker as described above. In such an example, each of the scFvs may be specific for the same target polypeptide or for different target polypeptides. Such multiple scFvs may be fused to the Fc portion of the scFc molecule separately at different fusion sites (such as the hinge region of the Fc or at the CH2 or CH3 region) or alternatively, a first scFv can be fused to the Fc portion with another scFv fused to the first scFv (and in case where each scFv is specific for a different target polypeptide, creating a bispecific binding molecule).

Single chain antibodies may be formed by linking heavy and light chain variable region (Fv region) fragments via an amino acid bridge (short peptide linker), resulting in a single polypeptide chain. Such single-chain Fvs (scFvs) have been prepared by fusing DNA encoding a peptide linker between DNAs encoding the two variable region polypeptides (VL and VH). The resulting antibody fragments can form dimers or higher oligomers, depending on such factors as the length of a flexible linker between the two variable domains (Kortt et al., Protein Engineering 10:423, 1997). In particular embodiments, two or more scFvs are joined by use of a chemical cross-linking agent.

ScFvs can be constructed by cloning the variable domains of a mAb showing interesting binding properties from hybridoma cells or by direct selection of scFv fragments with the desired specificity from immunized or naive phage libraries. Additionally, techniques developed for the production of single chain antibodies can be adapted to produce scFvs specific for a desired target polypeptide. Such techniques include those described in U.S. Pat. No. 4,946,778; Bird (Science 242:423, 1988); Huston et al. (Proc. Natl. Acad. Sci. USA 85:5879, 1988); and Ward et al. (Nature 334:544, 1989).

In certain preferred embodiments, a bispecific antibody in accordance with the present invention is a tandem single chain Fv (tascFv). For the tascFv molecule, two scFv molecules are constructed such that one scFv is amino terminal to the other one in a tandem configuration. This can be done in each orientation. Tandem scFv molecules can be prepared with a linker between the scFv entities. In some embodiments, the linker is a Gly-Ser linker comprising a series of glycine and serine residues, and optionally including additional amino acids. In other embodiments, the linker is a lambda stump or a CH1 stump, both of which are derived from the native sequence just after the V region in the Fab. In accordance with the present invention, a tascFv is further constructed as a fusion protein to contain to contain a single chain Fc (“tascFv-scFc”). In typical variations, the tascFv-scFc is constructed with the C-terminal scFv fused to the N-terminus of the single chain Fc component. The C-terminal scFv may be fused directly to an Fc hinge region of the scFc. In some alternative embodiments, the C-terminal scFv is fused to the scFc component via a linker (e.g., a Gly-Ser linker).

In other embodiments, a bispecific antibody in accordance with the present invention comprises an scFv at the N terminus of a single chain Fc and another at the C terminus of the single Fc (a “biscFv-scFc”). In some variations, the N terminal scFv is directly fused to the Fc hinge and with either a short or a long linker at the C terminus connecting to the second scFv. These linkers are typically Gly-Ser linkers.

Polynucleotides and Polypeptides and Methods of Producing the Same. The invention also includes polynucleotides encoding the scFc molecules of the invention, as well as individual components of such (e.g., the Fc portion or the binding entities) of the present invention. In some embodiments of the invention there are provided polynucleotides encoding an Fc domain of an antibody. The polynucleotides of the invention can be cloned into a vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences, for example, an internal ribosomal entry site. The expression vector can be introduced into a cell by transfection, for example.

The scFc molecules of the present invention include variants having single or multiple amino acid substitutions, deletions, additions, or replacements that retain the biological properties (e.g., effector function) of the molecules of the invention. Thus, the present invention encompasses scFc molecules comprising Fc portions that are based on amino acid sequence variants of the native Fc polypeptide sequences. These variants are prepared by introducing appropriate nucleotide changes into the DNA encoding the Fc or by in vitro synthesis of the desired Fc. Such variants include, for example, humanized variants of non-human Fc domains, as well as deletions from, or insertions or substitutions of, residues within particular amino acid sequences of an Fc domain. Any combination of deletion, insertion, and substitution can be made to arrive at the final construct, provided that the final construct possesses the desired characteristics. The amino acid changes also may alter post-translational processing of the target polypeptide, such as changing the number or position of glycosylation sites, introducing a membrane anchoring sequence into the constant domain or modifying the leader sequence of the native Fc.

DNA encoding the amino acid sequence variants of the scFc molecules of the present invention is prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the target polypeptide or by total gene synthesis. These techniques may utilize target polypeptide nucleic acid (DNA or RNA), or nucleic acid complementary to the target polypeptide nucleic acid. Oligonucleotide-mediated mutagenesis is a preferred method for preparing substitution, deletion, and insertion variants of target polypeptide DNA.

The CDNA or genomic DNA encoding the binding molecule (e.g., the Fc polypeptide) is inserted into a replicable vector for further cloning (amplification of the DNA) or for expression. Many vectors are available, and selection of the appropriate vector will depend on 1) whether it is to be used for DNA amplification or for expression of the encoded protein, 2) the size of the DNA to be inserted into the vector, and 3) the host cell to be transformed with the vector. Each vector contains various components depending on its function (amplification of DNA or expression of DNA) end the host cell for which it is compatible. The vector components generally include, but are not limited to, one or more of the following: a signal sequence, an origin of replication, one or more marker genes, a promoter, and a transcription termination sequence.

In general, the signal sequence may be a component of the vector, or it may be a part of the target polypeptide DNA that is inserted into the vector. Included within the scope of this invention are binding molecule polypeptides with any native signal sequence deleted and replaced with a heterologous signal sequence. The heterologous signal sequence selected should be one that is recognized and processed (e.g., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, Ipp, or heat-stable enterotoxin II leaders.

Expression and cloning vectors may, but need not, contain a polynucleotide sequence that enables the binding molecule polynucleotide to replicate in one or more selected host cells. Generally, in cloning vectors this sequence is one that enables the vector to replicate independently of the host chromosomal DNA, and includes origins of replication or autonomously replicating sequences. Such sequences are well known for a variety of microbes. The origin of replication from the plasmid pBR322 is suitable for most Gram-negative bacteria.

DNA may also be replicated by insertion into the host genome. This is readily accomplished using Bacillus species as hosts, for example, by including in the vector a DNA sequence that is complementary to a sequence found in Bacillus genomic DNA. Transfection of Bacillus with this vector results in homologous recombination with the genome and insertion of the target polypeptide DNA. However, the recovery of genomic DNA encoding the binding molecule polypeptide is more complex than that of an exogenously replicated vector because restriction enzyme digestion is required to excise the target polypeptide DNA. Similarly, DNA also can be inserted into the genome of vertebrate and mammalian cells by conventional methods.

Expression and cloning vectors should contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g. ampicillin, neomycin, methotrexate, or tetracycline, (b) complement auxotrophic deficiencies; or (c) supply critical nutrients not available from complex media, e.g. the gene encoding D-alanine racemase for Bacilli.

Expression and cloning vectors will usually contain a promoter that is recognized by the host organism and is operably linked to the Fc polypeptide nucleic acid. Promoters are untranslated sequences located upstream (5′) to the start codon of a structural gene (generally within about 100 to 1000 bp) that control its transcription and translation. Such promoters typically fall into two classes, inducible and constitutive. Inducible promoters are promoters that initiate increased levels of transcription from DNA under their control in response to some change in culture conditions, e.g. the presence or absence of a nutrient or a change in temperature.

Construction of suitable vectors containing one or more of the above listed components employs standard ligation techniques. Isolated plasmids or DNA fragments are cleaved, tailored, and relegated in the form desired to generate the plasmids required.

Suitable host cells for expressing binding molecule of the present invention are microbial cells such as yeast, fungi, insect and prokaryotes. Suitable prokaryotes include eubacteria, such as Gram-negative or Gram-positive organisms, for example, E. coli, Bacilli such as B. subtilis, Pseudomonas species such as P. aeruginosa, Salmonella typhimurium, or Serratia marcescans. One preferred E. coli cloning host is E. coli 294 (American Type Cell Culture, Manassas, Va. ATCC 31,446), although other strains such as E. coli B, E. coli .sub.X 1776 (ATCC 31,537), E. coli RV308 (ATCC 31,608) and E. coli W3110 (ATCC 27,325) are suitable.

Host cells of the invention also include any insect expression cell line known, such as for example, Spodoptera frugiperda cells.

The expression cell lines may also be yeast cell lines, such as, for example, Saccharomyces cerevisiae and Schizosaccharomyces pombe cells.

The expression cells may also be mammalian cells such as, for example, hybridoma cells (e.g., NS0 cells), Chinese hamster ovary cells (CHO), baby hamster kidney cells, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TRI cells, MRC 5 cells, and FS4 cells.

Expression cells may be engineered to provide an exogenous cellular activity or to remove an endogenous cellular activity. One non-limiting example includes the addition of a sialyltransferase gene to a cell to increase the sialylation of molecules expressed therefrom. Thus, such a cell can then be further manipulated to express an scFc molecule of the current invention and said cell will express a sialylated scFc molecule. In one embodiment, a CHO cell line is engineered to include express exogenous 2,6-sialyltransferase gene and to further express an scFc molecule of the current invention. Expression cells may be cultured in the presence of agents that modulate the cell's endogenous protein production and/or activity. In one example, a cell can be cultured in an altered cell culture process that includes one or more of: adding an alkanoic acid; altering the osmolarity or altering the cell culture temperature to control the amount of sialylic acid that the cell adds to a glycoprotein produced in the host cell. See e.g., U.S. Pat. No. 5,705,364.

These examples are illustrative rather than limiting. Preferably the host cell should secrete minimal amounts of proteolytic enzymes, and additional protease inhibitors may desirably be incorporated in the cell culture.

Host cells are transfected and preferably transformed with the above-described expression or cloning vectors of this invention and cultured in conventional nutrient media modified as appropriate for inducing promoters, selecting transformants, or amplifying the genes encoding the desired sequences.

Cells used to produce the binding molecules of the present invention are cultured in suitable media as described generally in Sambrook et al., (Molecular Cloning: A Laboratory Manual New York: Cold Spring Harbor Laboratory Press, 1989). Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

It is currently preferred that the bacterial host cells be cultured at temperatures from 37.deg.C. to 29.deg.C., although temperatures as low as 20.deg.C. may be suitable. Optimal temperatures will depend on the host cells, the Fc sequence and other parameters. 37.deg.C. is generally preferred.

Methods of purification are known in the art. In some embodiments of the invention, methods for purification include filtration, affinity column chromatography, cation exchange chromatography, anion exchange chromatography, and concentration. In general, soluble binding molecule polypeptides are recovered from recombinant cell culture to obtain preparations that are substantially homogeneous. As a first step, the culture medium or periplasmic preparation is centrifuged to remove particulate cell debris. Periplasmic preparations are obtained in conventional fashion, e.g. by freeze-thaw or osmotic shock methods. The membrane and soluble protein fractions are then separated. The Fc polypeptide is then purified from the soluble protein fraction. The following procedures are exemplary of suitable purification procedures: fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; protein A or protein G affinity matrix (e.g. Sepharose) columns; and hydrophobic interaction chromotography. More specifically, the filtration step preferably comprises ultrafiltration, and more preferably ultrafiltration and diafiltration. Filtration is preferably performed at least about 5-50 times, more preferably 10 to 30 times, and most preferably 14 to 27 times. Affinity column chromatography, may be performed using, for example, PROSEP Affinity Chromatography (Millipore, Billerica, Mass.). In a one embodiment, the affinity chromatography step comprises PROSEP-VA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The antibody product is preferably nanofiltered, for example, using a Pall DV 20 Nanofilter. The antibody product may be concentrated, for example, using ultrafiltration and diafiltration. The method may further comprise a step of size exclusion chromatography to remove aggregates. Sialylated Fc fractions can be isolated using affinity chromatography with immobilized Sambucus nigra lectin (Vector labs), followed by elution with lactose (See e.g., Shibuya, et al., Archives of Biochemistry and Biophysics, 254 (1): 1 (1987)).

Immunoconjugates and Derivatives

The scFc molecules of the present invention may be used alone or as immunoconjugates with a cytotoxic agent. In some embodiments, the agent is a chemotherapeutic agent. In some embodiments, the agent is a radioisotope, including, but not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, and fissionable nuclides such as Boron-10 or an Actinide. In other embodiments, the agent is a toxin or cytotoxic drug, including but not limited to ricin, modified Pseudomonas enterotoxin A, calicheamicin, adriamycin, 5-fluorouracil, and the like. Methods of conjugation of antibodies and binding molecules to such agents are known in the literature.

The scFc molecules of the present invention include those that are modified, e.g., by the covalent attachment of any type of other molecule such that covalent attachment does not prevent it from binding to its epitope. Examples of suitable covalent attachments include, but are not limited to fucosylated antibodies and fragments, sialylated antibodies and fragments, glycosylated antibodies and fragments, acetylated antibodies and fragments, pegylated antibodies and fragments, phosphorylated antibodies and fragments, and amidated antibodies and fragments. Multispecific binding scFc molecules of the present invention may themselves by derivatized by known protecting/blocking groups, proteolytic cleavage, linkage to a cellular ligand or other proteins, and the like.

Therapeutic Uses of the Binding molecules of the Invention

Methods of Treatment

A. General

An scFc molecule of the current invention can comprise one or more binding entities useful for treating disorders that are amenable to antibody therapies. Common diseases for antibody therapies include cancers, immune-related disorders, T-cell related disorders, metabolic diseases and neurodegenerative diseases, to name a few.

In another aspect, the present invention provides methods of treating disorders by administering to a person suffering from or suspected of suffering from a disorder an scFc molecule. Preferably, the administered scFc molecule will comprise one or more binding entities designed to target an antigen known or suspected to be involved in said disorder; at least one cytotoxic agent or a combination thereof. Administration amounts will typically be an amount effective for treating the disorder, though such will not always be the case, as is typical for clinical trials, for example. As one non-limiting example of a therapeutic use to treat a cancer, an scFc molecule comprising a binding entity directed towards PDGFR.beta. and/or a binding entity directed towards VEGF-A can be administered to a subject suffering from, or at an elevated risk of developing, a disease or disorder characterized by increased angiogenesis (an “neovascular disorder”).

In certain embodiments the scFc molecule is used in combination with a second antagonist. The second antagonist can be another antibody. Further, the second antagonist can be directed towards the same target as is the scFc molecule, or can be directed towards a distinct target, wherein its modulation is either known or suspected of being beneficial to treatment of an indication.

In each embodiments comprising the use of an scFc molecule antagonist in combination with a second antagonist, the scFc molecule and the second antagonist may be admininstered either simultaneously or separately (e.g., at different times and/or at separate administration sites). Accordingly, in certain variations comprising the simultaneous administration of an scFc molecule antagonist and a second antagonist, the second antagonist is a binding entity and is attached to the scFc molecule using a linker. This scFc bispecific binding molecule is useful in a method of administration of a composition comprising a first and a second antagonist. One non-limiting example includes an scFc bispecific binding molecule comprising (a) a linked binding entity that specially binds to the extracellular domain of PDGFR.beta. and neutralizes PDGFR.beta. activity and (b) a linked binding entity that specifically binds to VEGF-A and neutralizes VEGF-A activity. In particularly preferred embodiments, administration of an scFc molecule antagonist and a second antagonist comprises administering a bispecific scFc that binds to and neutralizes both of a first target and a second target. In certain other embodiments comprising separate administration of an scFc molecule antagonist and a second antagonist, the first and second antagonists are administered sequentially. In such embodiments, the administration of each agent can be by the same or different routes of administration.

In each of the embodiments of the treatment methods described herein, an antagonist is delivered in a manner consistent with conventional methodologies associated with management of the disease or disorder for which treatment is sought. In accordance with the disclosure herein, an effective amount of the antagonist is administered to a subject in need of such treatment for a time and under conditions sufficient to prevent or treat the disease or disorder.

Subjects for administration of antagonists as described herein include patients at high risk for developing a particular disease or disorder and patients presenting with a particular disease or disorder. In certain embodiments, the subject has been diagnosed as having the disease or disorder for which treatment is sought. Further, subjects can be monitored during the course of treatment for any change in the disease or disorder (e.g., for an increase or decrease in clinical symptoms of the disease or disorder).

In prophylactic applications, pharmaceutical compositions or medicants are administered to a patient susceptible to, or otherwise at risk of, a particular disease in an amount sufficient to eliminate or reduce the risk or delay the outset of the disease. In therapeutic applications, compositions or medicants are administered to a patient suspected of, or already suffering from such a disease in an amount sufficient to cure, or at least partially arrest, the symptoms of the disease and its complications. An amount adequate to accomplish this is referred to as a therapeutically- or pharmaceutically-effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the invention, accepted screening methods may be employed to determine risk factors associated with specific disorders or to determine the status of an existing disorder identified in a subject. Such methods can include, for example, determining whether an individual has relatives who have been diagnosed with a particular disease. Screening methods can also include, for example, conventional work-ups to determine familial status for a particular disease known to have a heritable component. For example, various cancers are also known to have certain inheritable components. Inheritable components of cancers include, for example, mutations in multiple genes that are transforming (e.g., Ras, Raf, EGFR, cMet, and others), the presence or absence of certain HLA and killer inhibitory receptor (KIR) molecules, or mechanisms by which cancer cells are able to modulate immune suppression of cells like NK cells and T cells, either directly or indirectly (see, e.g., Ljunggren and Malmberg, Nature Rev. Immunol. 7:329-339, 2007; Boyton and Altmann, Clin. Exp. Immunol. 149:1-8, 2007). Toward this end, nucleotide probes can be routinely employed to identify individuals carrying genetic markers associated with a particular disease of interest. In addition, a wide variety of immunological methods are known in the art that are useful to identify markers for specific diseases. For example, various ELISA immunoassay methods are available and well-known in the art that employ monoclonal antibody probes to detect antigens associated with specific tumors. Screening may be implemented as indicated by known patient symptomology, age factors, related risk factors, etc. These methods allow the clinician to routinely select patients in need of the methods described herein for treatment. In accordance with these methods, inhibition of angiogenesis may be implemented as an independent treatment program or as a follow-up, adjunct, or coordinate treatment regimen to other treatments.

Pharmaceutical compositions as described herein may also be used in the context of combination therapy. The term “combination therapy” is used herein to denote that a subject is administered at least one therapeutically effective dose of an scFc molecule antagonist and another therapeutic agent. The scFc molecule antagonist may be, for example, a bispecific scFc molecule composition that binds and neutralizes two targets.

For example, in the context of cancer immunotherapy, an scFc molecule having PDGFR.beta. and/or VEGF-A antagonist activity can be used as an angiogenesis inhibition agent in combination with chemotherapy or radiation. PDGFR.beta. and/or VEGF-A antagonists can work in synergy with conventional types of chemotherapy or radiation. PDGFR.beta. and/or VEGF-A antagonists can further reduce tumor burden and allow more efficient killing by the chemotherapeutic.

ScFc molecules of the present invention can also be used in combination with immunomodulatory compounds including various cytokines and co-stimulatory/inhibitory molecules. These could include, but are not limited to, the use of cytokines that stimulate anti-cancer immune responses. For instance, the combined use of IL-2 and IL-12 shows beneficial effects in T-cell lymphoma, squamous cell carcinoma, and lung cancer. (See Zaki et al., J. Invest. Dermatol. 118:366-71, 2002; Li et al., Arch. Otolaryngol. Head Neck Surg. 127:1319-24, 2001; Hiraki et al., Lung Cancer 35:329-33, 2002.) For example, PDGFR.beta. and/or VEGF-A antagonists could be combined with reagents that co-stimulate various cell surface molecules found on immune-based effector cells, such as the activation of CD137. (See Wilcox et al., J. Clin. Invest. 109:651-9, 2002) or inhibition of CTLA4 (Chambers et al., Ann. Rev. Immunol. 19:565-94, 2001). Alternatively, PDGFR.beta. and/or VEGF-A antagonists could be used with reagents that induce tumor cell apoptosis by interacting with TRAIL-related receptors. (See, e.g., Takeda et al., J. Exp. Med. 195:161-9, 2002; Srivastava, Neoplasia 3:535-46, 2001.) Such reagents include TRAIL ligand, TRAIL ligand-Ig fusions, anti-TRAIL antibodies, and the like.

In other variations, an scFc molecule is used in combination with a monoclonal antibody therapy. The use of monoclonal antibodies, particularly antibodies directed against tumor-expressed antigens, is becoming a standard practice for many tumors including Non-Hodgkins lymphoma (rituximab or RITUXAN.R™.), forms of leukemia (gemtuzumab or MYLOTARG.R™.), breast cell carcinoma (trastuzumab or HERCEPTIN.R™.) and colon carcinoma (cetuximab or ERBITUX.R™.). One mechanism by which antibodies mediate an anti-cancer effect is through a process referred to as antibody-dependent cell-mediated cytotoxicity (ADCC) in which immune-based cells, including NK cells, macrophages and neutrophils kill those cells that are bound by the antibody complex. Examples of this type of treatment paradigm include the combination use of RITUXAN.R™. (rituximab) and either IL-2, IL-12, or IFN-.alpha. for the treatment of Hodgkin's and Non-Hodgkin's lymphoma (Keilholz et al., Leuk. Lymphoma 35:641-2, 1999; Ansell et al., Blood 99:67-74, 2002; Carson et al., Eur. J. Immunol. 31:3016-25, 2001; and Sacchi et al., Haematologica 86:951-8., 2001). Similarly, because an scFc molecule can comprise one or more binding entities shown to enhance proliferation and differentiation of hematopoietic and lymphoid cells, as well as NK cells, an scFc molecule of the present invention can be used therapeutically or clinically to enhance the enhance the activity and effectiveness of antibody therapy in human disease.

ScFc molecules of the current invention may be used in combination with cell adoptive therapy. One method used to treat cancer is to isolate anti-cancer effector cells directly from patients, expand these in culture to very high numbers, and then to reintroduce these cells back into patients. The growth of these effector cells, which include NK cells, LAK cells, and tumor-specific T-cells, requires cytokines such as IL-2 (Dudley et al., J. Immunother. 24:363-73, 2001). An scFc molecule comprising binding entities shown to have growth stimulatory properties on lymphocytes, may also be used to propagate these cells in culture for subsequent re-introduction into patients in need of such cells. Following the transfer of cells back into patients, methods are employed to maintain their viability by treating patients with cytokines such as IL-2 (Bear et al., Cancer Immunol. Immunother. 50:269-74, 2001; and Schultze et al., Br. J. Haematol. 113:455-60, 2001).

An scFc molecule of the current invention may be used in combination with tumor vaccines. The major objective of cancer vaccination is to elicit an active immune response against antigens expressed by the tumor. Numerous methods for immunizing patients with cancer antigens have been employed, and a variety of techniques are being used to amplify the strength of the immune response following antigen delivery (reviewed in Rosenberg, SA. (Ed.), Principles and practice of the biologic therapy of cancer, 3rd edition, Lippincott Williams & Wilkins, Philadelphia, PA, 2000). Methods in which an scFc molecule may be used in combination with a tumor vaccine include, but are not limited to, the delivery of autologous and allogeneic tumor cells that either express a target gene or in which an scFc molecule is delivered in the context of a adjuvant protein. Similarly, an scFc molecule can be delivered in combination with injection of purified tumor antigen protein, tumor antigen expressed from injected DNA, or tumor antigen peptides that are presented to effector cells using dendritic cell-based therapies. Examples of these types of therapies include the use of cytokines like IL-2 in the context of vaccination with modified tumor cells (Antonia et al., J. Urol. 167:1995-2000, 2002; and Schrayer et al., Clin. Exp. Metastasis 19:43-53, 2002), DNA (Niethammer et al., Cancer Res. 61:6178-84, 2001), and dendritic cells (Shimizu et al., Proc. Nat. Acad. Sci USA 96:2268-73, 1999). An scFc molecule can be used as an anti-cancer vaccine adjuvant.

Pharmaceutical compositions may be supplied as a kit comprising a container that comprises a therapeutic scFc molecule as described herein. A therapeutic composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a therapeutic composition. Such a kit may further comprise written information on indications and usage of the pharmaceutical composition.

B. Cancer Treatment

An scFc molecule can comprise one or more binding entities designed to treat any of the following disorders: carcinoma, a sarcoma, a glioma, a lymphoma, a leukemia, or a skin cancer. The carcinoma can be a skin, an esophageal, a gastric, a colonic, a rectal, a pancreatic, a lung, a breast, an ovarian, a urinary bladder, an endometrial, a cervical, a testicular, a renal, an adrenal or a liver carcinoma. B-cell related disease may be an indolent form of B-cell lymphoma, an aggressive form of B-cell lymphoma, non-Hodgkin's lymphoma, a chronic lymphocytic leukemia, an acute lymphocytic leukemia, a Waldenstrom's macroglobulinemia, or a multiple myeloma. In addition, the B-cell related disease can be a human or a veterinary type of disease. Neovascular disorders amenable to treatment in accordance with the present invention include, for example, cancers characterized by solid tumor growth (e.g., pancreatic cancer, renal cell carcinoma (RCC), colorectal cancer, non-small cell lung cancer (NSCLC), and gastrointestinal stromal tumor (GIST)) as well as various neovascular ocular disorders (e.g., age-related macular degeneration, diabetic retinopathy, iris neovascularization, and neovascular glaucoma). A T-cell related disease may be a human or veterinary T-cell leukemia, skin psoriasis, psoriatic arthritis or mycosis fungoides. A metabolic disease can be an amyloidosis. A neurodegenerative disease can be an Alzheimer's disease.

A tumor-associated antigen can be associated with any type of disease. By way of example only, an scFc molecule can comprise one or more binding entities, each individually directed to one of the following: CD2, CD3, CD8, CD10, CD19, CD20, CD21, CD22, CD23, CD24, CD25, CD30, CD33, CD37, CD38, CD40, CD45Ro, CD48, CD52, CD55, CD59, CD70, CD74, CD80, CD86, CD138, CD147, HLA-DR, CEA, CSAp, CA-125, TAG-72, EFGR, HER2, HER3, HER4, IGF-IR, c-Met, PDGFR, MUC1, MUC2, MUC3, MUC4, TNFR1, TNFR2, NGFR, Fas (CD95), DR3, DR4, DR5, DR6, VEGF, PIGF, tenascin, ED-B fibronectin, PSMA, PSA, carbonic anhydrase IX, and IL-6. Tumor-associated markers have been categorized by Herberman (see, e. g, Immunodiagnosis of Cancer, in THE CLINICAL BIOCHEMISTRY OF CANCER, Fleisher (ed.), American Association of Clinical Chemists, 1979) in a number of categories. Occasionally, a sub-unit of a tumor-associated marker is advantageously used to raise antibodies having higher tumor-specificity, e. g., the beta-subunit of human chorionic gonadotropin (HCG) or the gamma region of carcinoembryonic antigen (CEA), which stimulate the production of antibodies having a greatly reduced cross-reactivity to non-tumor substances as disclosed in U.S. Pat. Nos. 4,361,644 and 4,444,744. Markers of tumor vasculature (e. g., VEGF, PDGFR, PIGF, and ED-B fibronectin), of tumor necrosis, of membrane receptors (e. g., folate receptor, EGFR), of transmembrane antigens (e. g., PSMA), and of oncogene products can also serve as suitable tumor-associated targets for an scFc molecule. Markers of normal cell constituents which are overexpressed on tumor cells, such as B-cell complex antigens, as well as cytokines expressed by certain tumor cells (e. g., IL-2 receptor in T-cell malignancies and IL-6 expressed by certain tumor cells and also involved in cachexia related, it has been proposed, to an inflammatory process) are also suitable targets for the antibodies and antibody fragments of this invention. See, for example, Trikha et al., Clin Cancer Res.; 9: 4653-65 (2003).

Also of use are scFc molecules against markers or products of oncogenes, or against angiogenesis factors. VEGF antibodies are described in U.S. Pat. Nos. 6,342,221; 5,965,132; and 6,004,554. ED-B fibronectin antibodies are disclosed in Santimaria, M. et al., Clin. Cancer Res. 9 (2): 571-579, 2003; WO 97/45544A1; WO 03/055917A2; WO 01/83816A2; WO 01/62298A2; WO 99/58570A2 ; WO 01/62800A1; and U. S. patent publication No. 20030045681A1. Antibodies against certain immune response modulators, such as antibodies to CD40, are described in Todryk et al., J. Immunol. Meth. 248: 139-147, 2001; and Turner et al., J. Immunol. 166: 89-94, 2001. Other antibodies suitable for combination therapy include anti-necrosis antibodies as described in Epstein et al., see e. g. , U.S. Pat. Nos. 5,019,368; 5,882,626; and 6,017,514. An example of a T cell marker for arthritic psoriasis is CD45Ro and is described by Veale, D. J. et al. in Ann. Rheum. Dis. 53 (7): 450-454,1994.

1. Types of Cancer

Table 1 below lists some cancers characterized by solid tumor formation, organized predominantly by target tissues.

TABLE 1
Exemplary Cancers Involving Solid Tumor Formation

1. Head and Neck cancer

a. Brain
b. Oral cavity
c. Orophyarynx
d. Nasopharynx
e. Hypopharynx
f. Nasal cavities and paranasal sinuses
g. Larynx
h. Lip

2. Lung cancers

a. Non-small cell carcinoma
b. Small cell carcinoma

3. Gastrointestinal Tract cancers

a. Colorectal cancer
b. Gastric cancer
c. Esophageal cancer
d. Anal cancer
e. Extrahepatic Bile Duct cancer
f. Cancer of the Ampulla of Vater
g. Gastrointestinal Stromal Tumor (GIST)

4. Liver cancer

a. Liver Cell Adenoma
b. Hepatocellular Carcinoma

5. Breast cancer
6. Gynecologic cancer

a. Cervical cancer
b. Ovarian cancer
c. Vaginal cancer
d. Vulvar cancer
e. Gestational Trophoblastic Neoplasia
f. Uterine cancer

7. Urinary Tract cancer

a. Renal cancer carcinoma
b. Prostate cancer
c. Urinary Bladder cancer
d. Penile cancer
e. Urethral cancer

8. Urinary Bladder cancer
9. Neurological Tumors

a. Astrocytoma and glioblastoma
b. Primary CNS lymphoma
c. Medulloblastoma
d. Germ Cell tumors
e. Retinoblastoma

10. Endocrine Neoplasms

a. Thyroid cancer
b. Pancreatic cancer

1) Islet Cell tumors

a) Insulinomas
b) Glucagonomas

c. Pheochromocytoma
d. Adrenal carcinoma
e. Carcinoid tumors
f. Parathyroid cancinoma
g. Pineal gland neoplasms

11. Skin cancers

a. Malignant melanoma
b. Squamous Cell carcinoma
c. Basal Cell carcinoma
d. Kaposi's Sarcoma

12. Bone cancers

a. Osteoblastoma
b. Osteochondroma
c. Osteosarcoma

13. Connective Tissue neoplasms

a. Chondroblastoma
b. Chondroma

14. Hematopoietic malignancies

a. Non-Hodgkin Lymphoma

1) B-cell lymphoma
2) T-cell lymphoma
3) Undifferentiated lymphoma

b. Leukemias

1) Chronic Myelogenous Leukemia
2) Hairy Cell Leukemia
3) Chronic Lymphocytic Leukemia
4) Chronic Myelomonocytic Leukemia
5) Acute Myelocytic Leukemia
6) Acute Lymphoblastic Leukemia

c. Myeloproliferative Disorders

1) Multiple Myeloma
2) Essential Thrombocythemia
3) Myelofibrosis with Myeloid Metaplasia
4) Hypereosinophilic Syndrome
5) Chronic Eosinophilic Leukemia
6) Polycythemia Vera

d. Hodgkin Lymphoma

15. Childhood Cancers

a. Leukemia and Lymphomas
b. Brain cancers
c. Neuroblastoma
d. Wilm's Tumor (nephroblastoma)
e. Phabdomyosarcoma
f. Retinoblastoma

16. Immunotherapeutically sensitive cancers

a. melanoma
b. kidney cancer
c. leukemias, lymphomas and myelomas
d. breast cancer
e. prostate cancer
f. colorectal cancer
g. cervical cancer
h. ovarian cancer
i. lung cancer

Some of the cancers listed above, including some of the relevant animal models for evaluating the effects of an scFc molecule on such cancers, are discussed in further detail below.

Chronic myeloid leukemia (CML) is a rare type of cancer affecting mostly adults. It is a cancer of granulocytes (one of the main types of white blood cells). In CML many granulocytes are produced and they are released into the blood when they are immature and unable to work properly. The production of other types of blood cells is also disrupted. Normally, white blood cells repair and reproduce themselves in an orderly and controlled manner, but in chronic myeloid leukemia the process gets out of control and the cells continue to divide and mature abnormally. The disease usually develops very slowly, which is why it is called ‘chronic’ myeloid leukemia. Because CML develops (progresses) slowly, it is difficult to detect in its early stages. The symptoms of CML are often vague and non-specific and are caused by the increased number of abnormal white blood cells in the bone marrow and the reduced number of normal blood cells: a feeling of fullness or a tender lump on the left side of the abdomen because of enlargement of the spleen. The effects of an scFc molecule for the treatment of chronic myeloid leukemia can be evaluated in a murine chronic myeloid leukemia model similar to that described in Ren, R., Oncogene. 2002 Dec 9;21(56):8629-42; Wertheim et al., Oncogene. 2002 Dec. 9; 21(56):8612-28; and Wolff et al., Blood. 2001 Nov 1;98(9):2808-16.

Multiple myeloma is a type of cancer that affects the plasma cells by causing their unregulated production. Myeloma cells tend to collect in the bone marrow and in the hard, outer part of bones. Myeloma cells can form a single mass, or tumor called a plasmacytoma or form many tumors, thus the disease is called multiple myeloma. Those suffering from multiple myeloma have an abnormally large number of identical plasma cells, and also have too much of one type of antibody. These myeloma cells and antibodies can cause a number of serious medical problems: (1) myeloma cells damage and weaken bones, causing pain and sometimes fractures; (2) hypocalcaemia, which often results in loss of appetite, nausea, thirst, fatigue, muscle weakness, restlessness, and confusion; (3) myeloma cells prevent the bone marrow from forming normal plasma cells and other white blood cells that are important to the immune system; (4) myeloma cells prevent the growth of new red blood cells, causing anemia; and (5) kidney problems. Symptoms of multiple myeloma depend on how advanced is the disease. In the earliest stage of the disease a patient may be asymptomatic. Symptoms include bone pain, broken bones, weakness, fatigue, weight loss, repeated infections, nausea, vomiting, constipation, problems with urination, and weakness or numbness in the legs. The effects of an scFc molecule designed to treat multiple myeloma can be evaluated in a multiple myeloma murine model similar to that described in Oyajobi et al., Blood. 2003 Jul. 1; 102(1):311-9; Croucher et al., J Bone Miner Res. 2003 Mar;18(3):482-92; Asosingh et al., Hematol J. 2000;1(5):351-6; and Miyakawa et al., Biochem Biophys Res Commun. 2004 Jan. 9; 313(2):258-62.

Lymphomas are a type of cancer of the lymphatic system. There are two main types of lymphoma. One is called Hodgkin's disease (named after Dr Hodgkin, who first described it). The other is called non-Hodgkin's lymphoma. There are about 20 different types of non-Hodgkin's lymphoma. In most cases of Hodgkin's disease, a particular cell known as the Reed-Stemberg cell is found in the biopsies. This cell is not usually found in other lymphomas, so they are called non-Hodgkin's lymphoma. Symptoms of a non-Hodgkin's lymphoma is a painless swelling of a lymph node in the neck, armpit or groin; night sweats or unexplained high temperatures (fever); loss of appetite, unexplained weight loss and excessive tiredness. The effects of an scFc molecule designed to treat a lymphoma, particularly a non-Hodgkin's lymphoma, can be evaluated in a murine non-Hodgkin's lymphoma model similar to that described in Ansell et al., Leukemia. 2004 Mar; 18(3):616-23; De Jonge et al., J Immunol. 1998 Aug. 1; 161(3):1454-61; and Slavin et al., Nature. 1978 Apr. 13; 272(5654):624-6.

The classification of Non-Hodgkin's lymphomas most commonly used is the REAL classification system (Ottensmeier, Chemico-Biological Interactions 135-136:653-664, 2001.) Specific immunological markers have been identified for classifications of lymphomas. For example, follicular lymphoma markers include CD20+, CD3−, CD10+, CD5−; small lymphocytic lymphoma markers include CD20+, CD3−, CD10−, CD5+, CD23+; marginal zone B cell lymphoma markers include CD20+, CD3−, CD10−, CD23−; diffuse large B cell lymphoma markers include CD20+, CD3−; mantle cell lymphoma markers include CD20+, CD3−, CD10−, CD5+, CD23+; peripheral T-cell lymphoma markers include CD20−, CD3+; primary mediastinal large B cell lymphoma markers include CD20+, CD3−, lymphoblastic lymphoma markers include CD20−, CD3+, Tdt+, and Burkitt's lymphoma markers include CD20+, CD3−, CD10+, CD5− (Decision Resourses, Non-Hodgkins Lymphoma, Waltham, Mass., February 2002).

Melanomas: Superficial spreading melanoma is the most common type of melanoma. About 7 out of 10 (70%) are this type. The most common place in women is on the legs, while in men it is more common on the trunk, particularly the back. They tend to start by spreading out across the surface of the skin: this is known as the radial growth phase. The melanoma will then start to grow down deeper into the layers of the skin, and eventually into the bloodstream or lymph system to other parts of the body. Nodular melanoma occurs most often on the chest or back. It tends to grow deeper into the skin quite quickly if it is not removed. This type of melanoma is often raised above the rest of the skin surface and feels like a bump. It may be very dark brown-black or black. Lentigo maligna melanoma is most commonly found on the face. It grows slowly and may take several years to develop. Acral melanoma is usually found on the palms of the hands, soles of the feet or around the toenails. Other very rare types of melanoma of the skin include amelanotic melanoma (in which the melanoma loses its pigment and appears as a white area) and desmoplastic melanoma (which contains fibrous scar tissue). Malignant melanoma can start in parts of the body other than the skin but this is very rare. The parts of the body that may be affected are the eye, the mouth, under the fingernails (known as subungual melanoma) the vulval or vaginal tissues, or internally. The effects of an scFc molecule designed to treat melanoma can be evaluated in a murine melanoma model similar to that described in Hermans et al., Cancer Res. 2003 Dec. 1; 63(23):8408-13; Ramont et al., Exp Cell Res. 2003 Nov. 15; 291(1):1-10; Safwat et al., J Exp Ther Oncol. 2003 Jul.-Aug. 3(4):161-8; and Fidler, I. J., Nat New Biol. 1973 Apr. 4; 242(118): 148-9.

Renal cell carcinoma, a form of kidney cancer that involves cancerous changes in the cells of the renal tubule. The first symptom is usually blood in the urine. The cancer metastasizes or spreads easily; most often spreading to the lungs and other organs. The effects of an an scFc molecule designed to treat melanoma can be evaluated in a murine renal cell carcinoma model similar to that described in Sayers et al., Cancer Res. 1990 Sep. 1; 50(17):5414-20; Salup et al., Immunol. 1987 Jan. 15; 138(2):641-7; and Luan et al., Transplantation. 2002 May 27; 73(10):1565-72.

Cervical cancer, also called cervical carcinoma, develops from abnormal cells on the surface of the cervix. Cervical cancer is usually preceded by dysplasia, precancerous changes in the cells on the surface of the cervix. These abnormal cells can progress to invasive cancer. Once the cancer appears it can progress through four stages. The stages are defined by the extent of spread of the cancer. There are two main types of cervical cancer: (1) squamous type (epidermoid cancer), which may be diagnosed at an early stage by a pap smear; and (2) adenocarcinoma, which is usually detected by a pap smear and pelvic exam. Later stages of cervical cancer cause abnormal vaginal bleeding or a bloodstained discharge at unexpected times, such as between menstrual periods, after intercourse, or after menopause. Abnormal vaginal discharge may be cloudy or bloody or may contain mucus with a bad odor. Advanced stages of the cancer may cause pain. The effects of an scFc molecule designed to treat cervical cancer can be evaluated in a murine cervical cancer model similar to that described in Ahn et al., Hum Gene Ther. 2003 Oct. 10; 14(15):1389-99; Hussain et al., Oncology. 1992;49(3):237-40; and Sengupta et al., Oncology. 1991 ;48(3):258-61.

Head and Neck tumors: Most cancers of the head and neck are of a type called carcinoma (in particular squamous cell carcinoma). Carcinomas of the head and neck start in the cells that form the lining of the mouth, nose, throat or ear, or the surface layer covering the tongue. However, cancers of the head and neck can develop from other types of cells. Lymphoma develops from the cells of the lymphatic system. Sarcoma develops from the supportive cells which make up muscles, cartilage or blood vessels. Melanoma starts from cells called melanocytes, which give colour to the eyes and skin. The symptoms of a head and neck cancer will depend on its location- for example, cancer of the tongue may cause some slurring of speech. The most common symptoms are an ulcer or sore area in the head or neck that does not heal within a few weeks; difficulty in swallowing, or pain when chewing or swallowing; trouble with breathing or speaking, such as persistent noisy breathing, slurred speech or a hoarse voice; a numb feeling in the mouth; a persistent blocked nose, or nose bleeds; persistent earache, ringing in the ear, or difficulty in hearing; a swelling or lump in the mouth or neck; pain in the face or upper jaw; in people who smoke or chew tobacco, pre-cancerous changes can occur in the lining of the mouth, or on the tongue. These can appear as persistent white patches (leukoplakia) or red patches (erythroplakia). They are usually painless but can sometimes be sore and may bleed (Cancerbacup Internet website). The effects of an scFc molecule designed for treating head and neck cancers can be evaluated in a murine head and neck tumor model similar to that described in Kuriakose et al., Head Neck. 2000 Jan. 22(1):57-63; Cao et al., Clin Cancer Res. 1999 Jul. 5(7):1925-34; Hier et al., Laryngoscope. 1995 October; 105(10):1077-80; Braakhuis et al., Cancer Res. 1991 Jan. 1; 51(1):211-4; Baker, S. R., Laryngoscope. 1985 January;95(1):43-56; and Dong et al., Cancer Gene Ther. 2003 Feb. 10(2):96-104.

Brain Cancer: Tumors that begin in brain tissue are known as primary tumors of the brain. Primary brain tumors are named according to the type of cells or the part of the brain in which they begin. The most common primary brain tumors are gliomas. They begin in glial cells. There are many types of gliomas. Astrocytomas arise from star-shaped glial cells called astrocytes. In adults, astrocytomas most often arise in the cerebrum. In children, they occur in the brain stem, the cerebrum, and the cerebellum. A grade III astrocytoma is sometimes called an anaplastic astrocytoma. A grade IV astrocytoma is usually called a glioblastoma multiforme. Brain stem gliomas occur in the lowest part of the brain. Ependymomas arise from cells that line the ventricles or the central canal of the spinal cord. Oligodendrogliomas arise from cells that make the fatty substance that covers and protects nerves. These tumors usually occur in the cerebrum. They grow slowly and usually do not spread into surrounding brain tissue. The symptoms of brain tumors depend on tumor size, type, and location. Symptoms may be caused when a tumor presses on a nerve or damages a certain area of the brain. They also may be caused when the brain swells or fluid builds up within the skull. These are the most common symptoms of brain tumors: Headaches; Nausea or vomiting; Changes in speech, vision, or hearing; Problems balancing or walking; Changes in mood, personality, or ability to concentrate; Problems with memory; Muscle jerking or twitching (seizures or convulsions); and Numbness or tingling in the arms or legs. The effects of an scFc molecule designed to treat brain cancer can be evaluated in a glioma animal model similar to that described in Schueneman et al., Cancer Res. 2003 Jul. 15; 63(14):4009-16; Martinet et al., Eur J Surg Oncol. 2003 May 29(4):351-7; Bello et al., Clin Cancer Res. 2002 Nov. 8(11):3539-48; Ishikawa et al., Cancer Sci. 2004 January;95(1):98-103; Degen et al., J Neurosurg. 2003 November;99(5):893-8; Engelhard et al., Neurosurgery. 2001 March;48(3):616-24; Watanabe et al., Neurol Res. 2002 Jul. 24(5):485-90; and Lumniczky et al., Cancer Gene Ther. 2002 Jan. 9(1):44-52.

Thyroid Cancer: Papillary and follicular thyroid cancers account for 80 to 90 percent of all thyroid cancers. Both types begin in the follicular cells of the thyroid. Most papillary and follicular thyroid cancers tend to grow slowly. Medullary thyroid cancer accounts for 5 to 10 percent of thyroid cancer cases. Anaplastic thyroid cancer is the least common type of thyroid cancer (only 1 to 2 percent of cases). The cancer cells are highly abnormal and difficult to recognize. This type of cancer is usually very hard to control because the cancer cells tend to grow and spread very quickly. Early thyroid cancer often does not cause symptoms. But as the cancer grows, symptoms may include: A lump, or nodule, in the front of the neck near the prominentia laryngea; Hoarseness or difficulty speaking in a normal voice; Swollen lymph nodes, especially in the neck; Difficulty swallowing or breathing; or Pain in the throat or neck. The effects of an scFc molecule designed for the treatment of thyroid cancer can be evaluated in a murine or rat thyroid tumor model similar to that described in Quidville et al., Endocrinology. 2004 May;145(5):2561-71 (mouse model); Cranston et at., Cancer Res. 2003 Aug. 15; 63(16):4777-80 (mouse model); Zhang et al., Clin Endocrinol (Oxf). 2000 June;52(6):687-94 (rat model); and Zhang et al., Endocrinology. 1999 May;140(5):2152-8 (rat model).

Liver Cancer: There are two different types of primary liver cancer. The most common kind is called hepatoma or hepatocellular carcinoma (HCC), and arises from the main cells of the liver (the hepatocytes). This type is usually confined to the liver, although occasionally it spreads to other organs. There is also a rarer sub-type of hepatoma called Fibrolamellar hepatoma. The other type of primary liver cancer is called cholangiocarcinoma or bile duct cancer, because it starts in the cells lining the bile ducts. Most people who develop hepatoma usually also have a condition called cirrhosis of the liver. Infection with either the hepatitis B or hepatitis C virus can lead to liver cancer, and can also be the cause of cirrhosis, which increases the risk of developing hepatoma. People who have a rare condition called haemochromatosis, which causes excess deposits of iron in the body, have a higher chance of developing hepatoma. Thus, an scF c molecule of the present invention may be used to treat, prevent, inhibit the progression of, delay the onset of, and/or reduce the severity or inhibit at least one of the conditions or symptoms associated with hepatocellular carcinoma. The effects of an scFc molecule designed to treat liver cancer can be evaluated in a hepatocellular carcinoma transgenic mouse model, which includes the overexpression of transforming growth factor-.alpha. (TFG-.alpha.) alone (Jhappan et al., Cell, 61:1137-1146 (1990); Sandgren et al., Mol. Cell Biol., 13:320-330 (1993); Sandgren et al., Oncogene, 4:715-724 (1989); and Lee et al., Cancer Res., 52:5162:5170 (1992)) or in combination with c-myc (Murakami et al., Cancer Res., 53:1719-1723 (1993), mutated H-ras (Saitoh et al., Oncogene, 5:1195-2000 (1990)), hepatitis B viral genes encoding HbsAg and HBx (Toshkov et al., Hepatology, 20:1162-1172 (1994) and Koike et al., Hepatology, 19:810-819 (1994)), SV40 large T antigen (Sepulveda et al., Cancer Res., 49:6108-6117 (1989) and Schirmacher et al., Am. J. Pathol., 139:231-241 (1991)) and FGF19 (Nicholes et al., American Journal of Pathology, 160(6):2295-2307 (June 2002)).

Lung cancer: The effects of an scFc molecule designed to treat a lung cancer can be evaluated in a human small/non-small cell lung carcinoma xenograft model. Briefly, human tumors are grafted into immunodecicient mice and these mice are treated with an scFc molecule alone or in combination with other agents which can be used to demonstrate the efficacy of the treatment by evaluating tumor growth (emati et al., Clin Cancer Res. 2000 May;6(5):2075-86; and Hu et al., Clin Cancer Res. 2004 Nov. 15; 10(22):7662-70).

2. Endpoints and Anti-tumor Activity for Solid Tumors

While each protocol may define tumor response assessments differently, the RECIST (Response evaluation Criteria in solid tumors) criteria is currently considered to be the recommended guidelines for assessment of tumor response by the National Cancer Institute (see Therasse et al., J. Natl. Cancer Inst. 92:205-216, 2000). According to the RECIST criteria tumor response means a reduction or elimination of all measurable lesions or metastases. Disease is generally considered measurable if it comprises lesions that can be accurately measured in atleast one dimension as >20 mm with conventional techniques or >10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truely non-measurable lesions (too small to accurately measure). Non-measureable disease includes pleural effusions, ascites, and disease documented by indirect evidence.

The criteria for objective status are required for protocols to assess solid tumor response. Representative criteria include the following: (1) Complete Response (CR) defined as complete disappearance of all measurable and evaluable disease. No new lesions. No disease related symptoms. No evidence of non-evaluable disease; (2) Partial Response (PR) defined as greater than or equal to 50% decrease from baseline in the sum of products of perpendicular diameters of all measurable lesions. No progression of evaluable disease. No new lesions. Applies to patients with at least one measurable lesion; (3) Progression defined as 50% or an increase of 10 cm.sup.2 in the sum of products of measurable lesions over the smallest sum observed using same techniques as baseline, or clear worsening of any evaluable disease, or reappearance of any lesion which had disappeared, or appearance of any new lesion, or failure to return for evaluation due to death or deteriorating condition (unless unrelated to this cancer); (4) Stable or No Response defined as not qualifying for CR, PR, or Progression. (See, Clinical Research Associates Manual, ibid.)

Additional endpoints that are accepted within the oncology art include overall survival (OS), disease-free survival (DFS), objective response rate (ORR), time to progression (TTP), and progression-free survival (PFS) (see, Guidance for Industry: Clinical Trial Endpoints for the Approval of Cancer Drugs and Biologics, April 2005, Center for Drug Evaluation and Research, FDA, Rockville, Md.)

3. Combination Cancer Therapy

Antibody therapy utilizes antigens that are selectively expressed on certain cell types. Antibody therapy has been particularly successful in cancer treatment because certain tumors either display unique antigens, lineage-specific antigens, or antigens present in excess amounts relative to normal cells. The development of monoclonal antibody (MAb) therapy has evolved from mouse hybridoma technology (Kohler et al., Nature 256:495-497, 1975), which had limited therapeutic utility due to an inability to stimulate human immune effector cell activity and production of human antimouse antibodies (HAMA; Khazaeli et al., J. Immunother. 15:42-52, 1994). Engineering chimeric antibodies which were less antigenic was achieved using human constant regions and mouse variable regions. These antibodies had increased effector functions and reduced HAMA responses (Boulianne et al., Nature 312:643-646, 1984). Human monoclonal antibodies have also developed using phage display technology (McCafferty et al., Nature 348:552-554, 1990), and more recently, transgenic mice carrying human Ig loci have been used to produce fully human monoclonal antibodies (Green, J. Immunol. Methods 231:11-23, 1999). For a review of monoclonal antibody therapy, see, Brekke et al., Nat. Rev. Drug Discov. 2:52-62, 2002.

One of the mechanisms associated with the anti-tumor activity of monoclonal antibody therapy is antibody dependent cellular cytotoxicity (ADCC). In ADCC, monoclonal antibodies bind to a target cell (e.g. cancer cell) and specific effector cells expressing receptors for the monoclonal antibody (e.g. NK cells, monocytes and granulocytes) bind the monoclonal antibody/target cell complex resulting in target cell death. As has been stated, an scFc molecule can be co-administered with a second antagonist and that second antagonist can be a MAb. The dose and schedule of an scFc molecule administration in combination with MAbs is based on the ability of the scFc molecule to effect parameters associated with differentation and functional activity of cell populations mediating ADCC, including but not limited to, NK cells, macrophages and neutrophils. These parameters can be evaluated using assays which measure NK, macrophage and neutrophil cell cytotoxicity or effector molecules essential to the ability of cells to implement ADCC (e.g., FasL, granzymes and perforin). An scFc molecule may also increase cytokine and chemokine production by NK cells when combined with MAb plus tumor cells (e.g. IFN.gamma.). Another mechanism associated with anti-tumor activity is phagocytosis of MAb-coated tumor cells. This mechanism is Fc receptor-dependent and has been shown to influence B depletion by anti-CD20 antibody (Uchida et al. J. Exp. Med. 199(12):1659-69, 2004). The dose and schedule of the MAbs is based on pharmacokinetic and toxicokinetic properties ascribed to the specific antibody co-administered, and should optimize these effects, while minimizing any toxicity that may be associated with administration of a therapy.

Combination therapy with an scFc molecule and a monoclonal antibody may be indicated when a first line treatment has failed and may be considered as a second line treatment. The present invention also provides using the combination as a first line treatment in patient populations that are newly diagnosed and have not been previously treated with anticancer agents (“de novo patients”) and patients that have not previously received any monoclonal antibody therapy (“naive patients”).

An scFc molecule is also useful in combination therapy with monoclonal antibodies in the absence of any direct antibody mediated ADCC of tumor cells. Antibodies that block an inhibitory signal in the immune system can lead to augmented immune responses. Examples include (1) antibodies against molecules of the B7R family that have inhibitory function such as, cytotoxic T lymphocyte-associated antigen 4 (CTLA-4), programmed death-1 (PD-1), B and T lymphocyte attenuator (BTLA); (2) antibodies against inhibitory cytokines like IL-10, TGF.beta.; and (3) antibodies that deplete or inhibit functions of suppressive cells like anti-CD25 or CTLA-4. For example, anti-CTLA4 MAbs in both mice and humans are thought to either suppress function of immune-suppressive regulatory T cells (Tregs) or inhibit the inhibitory signal transmitted through binding of CTLA-4 on T cells to B7-1 or B7-2 molecules on APCs or tumor cells. CTLA-4 is expressed transiently on the surface of activated T cells and constitutively expressed on Treg cells. Cross-linking CTLA-4 leads to an inhibitory signal on activated T cells, and antibodies against CTLA-4 block the inhibitory signal on T cells leading to sustained T cell activation (Phan et al., PNAS, 100:8372-8377, 2003.) In mouse models, anti-CTLA4 treatment leads to an increase in numbers of activated tumor-specific CD8 T cells and NK cells resulting in potent anti-tumor responses. An scFc molecule can be designed to target receptors that are expressed on these effector cells and such an scFc molecule may augment their effector function further by activating these cells through the targeted receptors. This can lead to more potent anti-tumor activity. Clinical trials where blocking antibodies against CTLA-4 are administered to patients are ongoing in melanoma, ovarian and prostate cancer. However, efficacy has been correlated to serious adverse events (see, US 2004/0241169), and combination therapy resulting in less toxic treatment would be advantageous. Table 2 is a non-exclusive list of monoclonal antibodies approved or being tested for which combination therapy with an scFc molecule is possible. ScFc molecules of the current invention can be designed to target the same antigens as do these MAbs, or to separate target antigens, wherein modulation of these separate targets is known or suspected to be effective in treating an indication

TABLE 2
Monoclonal Antibody Therapies for Use in Combination with an scFc Molecule

| Target | Drug Name | Clinical Indication | Company |
|---|---|---|---|
| IL-2Rα(CD25) | Zenapax | kidney transplant | Roche |
| IL-1R | AMG108 | osteoarthritis | Amgen |
| RANK-L | AMG162 | osteoporosis | Amgen |
| Blys | LympoSTAT-B | SLE, RA | HGS |
| CD40L (CD39) | initiatedAID | Celltech/IDEC | |
| TRAIL-R1 | HGS-ETR1 | cancers | HGS |
| TRAIL-R2 | HGS-ETR2 | solid tumors | HGS |
| CD30 | SGN30 | Hodgkins, NHL | Seattle Genetics |
| CD40 | SGN40 | MM | Seattle Genetics |
| HER2 | Herceptin | Breast cancer | Genentech |
| EGF-R | ABX-EGF | CRC, NSCLC, RCC | Abgenix |
| | EMD72000 | solid tumors | Merck |
| | MDX-214 | EGF-R-positive tumors | Medarex |
| | Erbitux | CRC | Imclone |
| VEGF-R | CDP791 | solid tumors | Celltech |
| PDGF-R | CDP860 | solid tumors | Celltech/ZymoGenetics |
| CD11a(αL) | Raptiva | psoriasis | Genentech |
| α4-integrin | Antegrin | CD, MS | PDL, Biogen-IDEC |
| α4β7 integrin | MLM02 | CD, UC | Millenium |
| α5β3 integrin | Vitaxin | psoriasis, prostate cancer | AME/Lilly |
| CD2 (LFA3/Fc) | Amevive | psoriasis | Biogen/IDEC |
| CD152 | CTLA-4/Ig | RA | Bristol Meyers |
| CD152 | CTLA-4 | cancers | Medarex |
| CD49a | Integrin α1 | RA/Lupus | Biogen/IDEC |
| CD49e | Integrin α5 | cancers | Protein Design Labs |
| MUC1 | | | Theragyn |
| MUC18 (TIM-like) | ABX-MA1 | melanoma | |
| TAG-72 Mucin | Anatumomab | cancers | |
| CD3 | Ecromeximab | melanoma | Kyowa Hakko |
| | TRX4 | typeI IDDM | TolerRx |
| | Nuvion | UC | PDL |
| | OrthoCloneOKT3 | organ transplant | Ortho biotech |
| CD4 | HuMax-CD4 | T-cell lymphoma | GenMab |
| CD19 | MT103 | NHL | Medimmune |
| CD64(Fc GR1) | AntiCD64 | cancers | Medarex |
| SIGLECs: | | | |
| CD33 | MyloTarg | AML | Celltech/Wyeth |
| | ZAmyl | AML | Protein Design Labs |
| CD22 | lymphocide | NHL, AID | Immunomedics |
| CEA | CEA-Cide | cancers | Immunomedics |
| CD20 | Rituxan | NHL | Genentech |
| CD52 | Campath | MS, NHL, T-cell lymphoma | Genzyme, IDEX |
| CD44 | Bivatuzumab | cancers | Boehringer Ingelheim |
| CD23 (Fc Ep R) | IDEC152 | allerhic asthma, rhinitis | Biogen/IDEC |
| LRR: | | | |
| CD14 | ICOSIC14 | sepsis | ICOS |
| EpCAM | Panorex | colorectal cancer | Centocor |
| Lewis-Y—Ag | SGN15 | cancers | Seattle Genetics |
| CD80 | B7.1 | psoriasis/NHL | Biogen/IDEC |

b. Tyrosine Kinase Inhibitors in Combination with an scFc Molecule

In some embodiments, an scFc molecule is used in combination with a tyrosine kinase inhibitor. Tyrosine kinases are enzymes that catalyze the transfer of the .gamma.phosphate group from the adenosine triphosphate to target proteins. Tyrosine kinases can be classified as receptor and nonreceptor protein tyrosine kinases. They play an essential role in diverse normal cellular processes, including activation through growth receptors and affect proliferation, survival and growth of various cell types. Additionally, they are thought to promote tumor cell proliferation, induce anti-apoptotic effects and promote angiogenesis and metastasis. In addition to activation through growth factors, protein kinase activation through somatic mutation is a common mechanism of tumorigenesis. Some of the mutations identified are in B-Raf kinase, FLt3 kinase, BCR-ABL kinase, c-KIT kinase, epidermal growth factor (EGFR) and PDGFR pathways. The Her2, VEGFR and c-Met are other significant receptor tyrosine kinase (RTK) pathways implicated in cancer progression and tumorigenesis. Because a large number of cellular processes are initiated by tyrosine kinases, they have been identified as key targets for inhibitors.

Tyrosine kinase inhibitors (TKIs) are small molecules that act inside the cell, competing with adenosine triphosphate (ATP) for binding to the catalytic tyrosine kinase domain of both receptor and non-receptor tyrosine kinases. This competitive binding blocks initiation of downstream signaling leading to effector functions associated with these signaling events like growth, survival, and angiogenesis. Using a structure and computational approach, a number of compounds from numerous medicial chemistry combinatorial libraries was identified that inhibit tyrosine kinases. Most TKIs are thought to inhibit growth of tumors through direct inhibition of the tumor cell or through inhibition of angiogenesis. Moreover, certain TKIs affect signaling through the VEGF family receptors, including sorafenib and sunitinib. In some cases TKIs have been shown to activate functions of dendritic cells and other innate immune cells, like NK cells. This has been recently reported in animal models for imatinib. Imatinib is a TKI that has shown to enhance killer activity by dendritic cells and NK cells (for review, see Smyth et al., NEJM 354:2282, 2006).

BAY 43-9006 (sorafenib, Nexavar.R™.) and SUI 1248 (sunitinib, Sutent.R™.) are two such TKIs that have been recently approved for use in metastatic renal cell carcinoma (RCC). A number of other TKIs are in late and early stage development for treatment of various types of cancer. Other TKIs include, but are not limited to: Imatinib mesylate (Gleevec.R™., Novartis); Gefitinib (Iressa.R™ . . . R™., AstraZeneca); Erlotinib hydrochloride (Tarceva.R™., Genentech); Vandetanib (Zactima.R™., AstraZeneca), Tipifamib (Zamestra.R™., Janssen-Cilag); Dasatinib (Sprycel.R™., Bristol Myers Squibb); Lonafarnib (Sarasar.R™., Schering Plough); Vatalanib succinate (Novartis, Schering AG); Lapatinib (Tykerb.R™., GlaxoSmithKline); Nilotinib (ovartis); Lestaurtinib (Cephalon); Pazopanib hydrochloride (GlaxoSmithKline); Axitinib (Pfizer); Canertinib dihydrochloride (Pfizer); Pelitinib (ational Cancer Institute, Wyeth); Tandutinib (Millennium); Bosutinib (Wyeth); Semaxanib (Sugen, Taiho); AZD-2171 (AstraZeneca); VX-680 (Merck, Vertex); EXEL-0999 (Exelixis); ARRY-142886 (Array BioPharma, AstraZeneca); PD-0325901 (Pfizer); AMG-706 (Amgen); BIBF-1120 (Boehringer Ingelheim); SU-6668 (Taiho); CP-547632 (OSI); (AEE-788 (Novartis); BMS-582664 (Bristol-Myers Squibb); JNK-401 (Celgene); R-788 (Rigel); AZD-1152 HQPA (AstraZeneca); NM-3 (Genzyme Oncology); CP-868596 (Pfizer); BMS-599626 (Bristol-Myers Squibb); PTC-299 (PTC Therapeutics); ABT-869 (Abbott); EXEL-2880 (Exelixis); AG-024322 (Pfizer); XL-820 (Exelixis); OSI-930 (OSI); XL-184 (Exelixis); KRN-951 (Kirin Brewery); CP-724714 (OSI); E-7080 (Eisai); HKI-272 (Wyeth); CHIR-258 (Chiron); ZK-304709 (Schering AG); EXEL-7647 (Exelixis); BAY-57-9352 (Bayer); BIBW-2992 (Boehringer Ingelheim); AV-412 (AVEO); YN-968D1 (Advenchen Laboratories); Midostaurin (Novartis); Perifosine (AEtema Zentaris, Keryx, National Cancer Institute); AG-024322 (Pfizer); AZD-1152 (AstraZeneca); ON-01910Na (Onconova); and AZD-0530 (AstraZeneca).

c. Chemotherapy Combinations

In certain embodiments, an scFc molecule is administered in combination with one or more chemotherapeutic agents. Chemotherapeutic agents have different modes of actions, for example, by influencing either DNA or RNA and interfering with cell cycle replication. Examples of chemotherapeutic agents that act at the DNA level or on the RNA level are anti-metabolites (such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea); alkylating agents (such as Melphalan, Busulfan, Cis-platin, Carboplatin, Cyclophosphamide, Ifosphamide, Dacarabazine, Procarbazine, Chlorambucil, Thiotepa, Lomustine, Temozolamide); anti-mitotic agents (such as Vinorelbine, Vincristine, Vinblastine, Docetaxel, Paclitaxel); topoisomerase inhibitors (such as Doxorubincin, Amsacrine, Irinotecan, Daunorubicin, Epirubicin, Mitomycin, Mitoxantrone, Idarubicin, Teniposide, Etoposide, Topotecan); antibiotics (such as actinomycin and bleomycin); asparaginase; anthracyclines or taxanes.

d. Radiotherapy Combinations

In some variations, an scFc molecule is administered in combination with radiotherapy. Certain tumors can be treated with radiation or radiopharmaceuticals. Radiation therapy is generally used to treat unresectable or inoperable tumors and/or tumor metastases. Radiotherapy is typically delivered in three ways. External beam irradiation is administered at distance from the body and includes gamma rays (60Co) and X-rays. Brachytherapy uses sources, for example .sup.60Co, .sup.137Cs, .sup.1921r, or .sup.1251, with or in contact with a target tissue.

e. Hormonal Agent Combinations

In some embodiments, an scFc molecule is administered in combination with a hormone or anti-hormone. Certain cancers are associated with hormonal dependency and include, for example, ovarian cancer, breast cancer, and prostate cancer. Hormonal-dependent cancer treatment may comprise use of anti-androgen or anti-estrogen compounds. Hormones and anti-hormones used in cancer therapy include Estramustine phosphate, Polyestradiol phosphate, Estradiol, Anastrozole, Exemestane, Letrozole, Tamoxifen, Megestrol acetate, Medroxyprogesterone acetate, Octreotide, Cyproterone acetate, Bicaltumide, Flutamide, Tritorelin, Leuprorelin, Buserelin and Goserelin.

C. Immune System Dysregulation and Treatments Thereof.

Diseases of the immune system are significant healthcare problems that are growing at epidemic proportions. As such, they require novel, aggressive approaches to the development of new therapeutic agents. Standard therapy for autoimmune disease has been high dose, long-term systemic corticosteroids and immunosuppressive agents. The drugs used fall into three major categories: (1) glucocorticoids, such as prednisone and prednisolone; (2) calcineurin inhibitors, such as cyclosporine and tacrolimus; and (3) antiproliferative/antimetabolic agents such as azathioprine, sirolimus, and mycophenolate mofetil. Although these drugs have met with high clinical success in treating a number of autoimmune conditions, such therapies require lifelong use and act nonspecifically to suppress the entire immune system. The patients are thus exposed to significantly higher risks of infection and cancer. The calcineurin inhibitors and steroids are also nephrotoxic and diabetogenic, which has limited their clinical utility.

In addition to the conventional therapies for autoimmune disease, monoclonal antibodies and soluble receptors that target cytokines and their receptors have shown efficacy in a variety of autoimmune and inflammation diseases such as rheumatoid arthritis, organ transplantation, and Crohn's disease. Some of the agents include infliximab (REMICADE) and etanercept (ENBREL) that target tumor necrosis factor (TNF), muromonab-CD3 (ORTHOCLONE OKT3) that targets the T cell antigen CD3, and daclizumab (ZENAPAX) that binds to CD25 on activated T cells, inhibiting signaling through this pathway. While efficacious in treating certain inflammatory conditions, use of these drugs has been limited by side effects including the “cytokine release syndrome” and an increased risk of infection.

Passive immunization with intravenous immunoglobulin (IVIG) was licensed in the United States in 1981 for replacement therapy in patients with primary antibody deficiencies. IVIG is obtained from the plasma of large numbers (10,000-20,000) of healthy donors by cold ethanol fractionation. Commonly used IVIG preparations include Sandoglobulin, Flebogamma, Gammagard, Octagam, and Vigam S.

Subsequent investigation showed that IVIG was also effective in ameliorating autoimmune symptoms in Kawasaki's disease and immune thrombocytopenia purpura. IVIG has also been shown to reduce inflammation in adult dermatomyositis, Guillian-Barre syndrome, chronic inflammatory demyelinating polyneuropathies, multiple sclerosis, vasculitis, uveitis, myasthenia gravis, and in the Lambert-Eaton syndrome. Numerous mechanisms have been proposed to explain the mode of action of IVIG, including regulation of Fc gamma receptor expression, increased clearance of pathogenic antibodies due to saturation of the neonatal Fc receptor FcRn, attenuation of complement-mediated damage, and modulation of T and B cells or the reticuloendothelial system. Since Fc domains purified from IVIG are as active as intact IgG in a number of in vitro and in vivo models of inflammation, it is well accepted that the anti-inflammatory properties of IVIG reside in the Fc domain of the IgG. In general, efficacy is seen when only large amounts of IVIG are infused into a patient, with an average dose of 2 g/kg/month used in autoimmune disease.

The common (1-10% of patients) side effects of IVIG treatment include flushing, fever, myalgia, back pain, headache, nausea, vomiting, arthralgia, and dizziness. Uncommon (0.1-1% of patients) side effects include anaphylaxis, aseptic meningitis, acute renal failure, haemolytic anemia, and eczema. Although IVIG is generally considered safe, the pooled human plasma source is considered to be risk factor for transfer of infectious agents. Thus, the use of IVIG is limited by its availability, high cost ($100/gm, including infusion cost), and the potential for severe adverse reactions. Thus, it would be significantly advantageous to develop a therapeutic that offered the efficacy of IVIG without the numerous issues described above (undue side effects and cost/availability issues).

The scFc molecules of the invention, and in particular the single chain Fc portion itself (namely, scFc10.1, scFc10.2 and scFc10.3) address the shortcomings of the conventional therapies discussed above. It was surprisingly discovered that an scFc embodiment of the present invention could be useful in the treatment of autoimmune diseases. As described in Example 5 below, these scFcs were tested in two assays for inhibitory activity in immune complex assays. Specifically, scFc10.1 competitively blocked immune complex mediated secretion of IL-6 TNF-alpha, MCP-1, and IL-13 from murine MC/9 mast cells (scFc10.3 also showed some inhibitory activity, but was less active than scFc10.1). In contrast, the scFc10.2, containing the mutated hinge region described in Example 2, was inactive. These data suggested that the scFc10.1 bound to cell surface Fc receptors and blocked their interaction with extracellular immune complexes, thus preventing cytokine release. Accordingly, the scFc molecules of the invention can act alone as a therapeutic to treat immune diseases or may be used as a fusion partner with target-specific scFv molecules or tandem pairs of scFv molecules to form potent multispecific binding molecule drug candidates.

An scFc molecule can further comprise one or more binding entities designed for treating an autoimmune disease or other immune disorder. A non-limiting example of suitable antigen targets for treating immune systems disorders includes, IL-17 cytokine family, (IL-17A, IL-17B, IL-17C, IL-17D, IL-17, IL-17E, IL-17F), IL-17 receptor family, (IL-17RA, IL-17RB, IL-17RC, IL-17RD, IL-17RE), IL-23 cytokine family, IL-23 receptor family, CLA family, IL-31 cytokine family, IL-31 receptor family, IL-21 cytokine family, IL-21 receptor family, IL-2 cytokine family, RANTES cytokine family, TNF cytokine family, BlyS, TACI, IL-6 cytokine family, IL-8 cytokine family, IL-13 family, IL-12 cytokine family, IL-I family CD28-B7 family, CD40, and IL-2 family.

As such, the present invention concerns compositions and methods useful for the diagnosis and treatment of immune related disease in mammals, including humans. The present invention is based on the identification of scFc molecules which inhibit the immune response in mammals and may be used to treat inflammatory and immune diseases or conditions such as acute or chronic inflammation, ulcerative colitis, chronic bronchitis, asthma, emphysema, myositis, polymyositis, immune dysregulation diseases, cachexia, septicemia, atherosclerosis, psoriasis, psoriatic arthritis, atopic dermatitis, inflammatory skin conditions, rheumatoid arthritis, inflammatory bowel disease (IBD), Crohn's Disease, diverticulosis, pancreatitis, type I diabetes (IDDM), pancreatic cancer, pancreatitis, Graves Disease, colon and intestinal cancer, autoimmune disease, sepsis, organ or bone marrow transplant rejection; inflammation due to endotoxemia, trauma, surgery or infection; amyloidosis; splenomegaly; graft versus host disease; and where inhibition of inflammation, immune suppression, reduction of proliferation of hematopoietic, immune, inflammatory or lymphoid cells, macrophages, T-cells (including Th1 and Th2 cells), suppression of immune response to a pathogen or antigen. Immunotherapy of autoimmune disorders using antibodies which target B-cells is described in PCT Application Publication No. WO 00/74718. Exemplary autoimmune diseases are acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, parnphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, and fibrosing alveolitis.

Inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g., psoriasis, arthritis, rheumatoid arthritis, multiple sclerosis, inflammatory bowel disease and the like), septic shock and multiple organ failure. Importantly, these diverse disease states share common inflammatory mediators. The collective diseases that are characterized by inflammation have a large impact on human morbidity and mortality. Therefore it is clear that the antibodies of the present invention could have crucial therapeutic potential for a vast number of human and animal diseases, from asthma and allergy to autoimmunity and septic shock.

Arthritis, including osteoarthritis, rheumatoid arthritis, arthritic joints as a result of injury, and the like, are common inflammatory conditions which would benefit from the therapeutic use of the binding molecules of the present invention (such as the scFc of the invention). For example, rheumatoid arthritis (RA) is a systemic disease that affects the entire body and is one of the most common forms of arthritis. It is characterized by the inflammation of the membrane lining the joint, which causes pain, stiffniess, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

Rheumatoid arthritis (RA) is an immune-mediated disease particularly characterized by inflammation and subsequent tissue damage leading to severe disability and increased mortality. A variety of cytokines are produced locally in the rheumatoid joints. Numerous studies have demonstrated that IL-1 and TNF-alpha, two prototypic pro-inflammatory cytokines, play an important role in the mechanisms involved in synovial inflammation and in progressive joint destruction. Indeed, the administration of TNF-alpha and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2(2): 135-149, 2002).

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995).

The administration of the scFc binding molecules of the invention to these CIA model mice is used to evaluate the use of such a binding molecule as a therapeutic useful in ameliorating symptoms and altering the course of disease. By way of example and without limitation, the injection of 10-200 .micro.g of such an antibody fragment of the present invention per mouse (one to seven times a week for up to but not limited to 4 weeks via s.c., i.p., or i.m route of administration) can significantly reduce the disease score (paw score, incidence of inflammation, or disease). Depending on the initiation of administration (e.g. prior to or at the time of collagen immunization, or at any time point following the second collagen immunization, including those time points at which the disease has already progressed), such antibody fragments can be efficacious in preventing rheumatoid arthritis, as well as preventing its progression.

2. Endotoxemia

Endotoxemia is a severe condition commonly resulting from infectious agents such as bacteria and other infectious disease agents, sepsis, toxic shock syndrome, or in immunocompromised patients subjected to opportunistic infections, and the like. Therapeutically useful of anti-inflammatory proteins, such as antibodies of the invention, could aid in preventing and treating endotoxemia in humans and animals. Such antibody fragments could serve as a valuable therapeutic to reduce inflammation and pathological effects in endotoxemia.

Lipopolysaccharide (LPS) induced endotoxemia engages many of the proinflammatory mediators that produce pathological effects in the infectious diseases and LPS induced endotoxemia in rodents is a widely used and acceptable model for studying the pharmacological effects of potential pro-inflammatory or immunomodulating agents. LPS, produced in gram-negative bacteria, is a major causative agent in the pathogenesis of septic shock (Glausner et at., Lancet 338:732, 1991). A shock-like state can indeed be induced experimentally by a single injection of LPS into animals. Molecules produced by cells responding to LPS can target pathogens directly or indirectly. Although these biological responses protect the host against invading pathogens, they may also cause harm. Thus, massive stimulation of innate immunity, occurring as a result of severe Gram-negative bacterial infection, leads to excess production of cytokines and other molecules, and the development of a fatal syndrome, septic shock syndrome, which is characterized by fever, hypotension, disseminated intravascular coagulation, and multiple organ failure (Dumitru et al. Cell 103:1071-1083, 2000).

These toxic effects of LPS are mostly related to macrophage activation leading to the release of multiple inflammatory mediators. Among these mediators, TNF appears to play a crucial role, as indicated by the prevention of LPS toxicity by the administration of neutralizing anti-TNF antibodies (Beutler et al., Science 229:869, 1985). It is well established that 1 .micro.g injection of E. coli LPS into a C57B1/6 mouse will result in significant increases in circulating IL-6, TNF-alpha, IL-1, and acute phase proteins (for example, SAA) approximately 2 hours post injection. The toxicity of LPS appears to be mediated by these cytokines as passive immunization against these mediators can result in decreased mortality (Beutler et al., Science 229:869, 1985). The potential immunointervention strategies for the prevention and/or treatment of septic shock include anti-TNF mAb, IL-1 receptor antagonist, LIF, IL-10, and G-CSF.

The administration of antibody fragments of the invention to an LPS-induced model may be used to evaluate the use of such antibody fragments to ameliorate symptoms and alter the course of LPS-induced disease. Moreover, results showing inhibition of immune response by such antibody fragments of the invention provide proof of concept that such antibody fragments can also be used to ameliorate symptoms in the LPS-induced model and alter the course of disease. The model will show induction of disease specific cytokines by LPS injection and the potential treatment of disease by such antibody fragments. Since LPS induces the production of pro-inflammatory factors possibly contributing to the pathology of endotoxemia, the neutralization of pro-inflammatory factors by antibody fragments of the invention can be used to reduce the symptoms of endotoxemia, such as seen in endotoxic shock.

Inflammatory Bowel Disease IBD. In the United States approximately 500,000 people suffer from Inflammatory Bowel Disease (IBD) which can affect either colon and rectum (Ulcerative colitis) or both, small and large intestine (Crohn's Disease). The pathogenesis of these diseases is unclear, but they involve chronic inflammation of the affected tissues. Antibody fragments of the invention could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining. Treatments including corticosteroids, immunosuppressives (eg. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of antibody fragments of the invention to these TNBS or DSS models can be used to evaluate the use such antibody fragments to ameliorate symptoms and alter the course of gastrointestinal disease.

4. Psoriasis

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. The antibody fragments of the invention could serve as a valuable therapeutic to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

In addition to other disease models described herein, the activity of antibody fragments of the invention on inflammatory tissue derived from human psoriatic lesions can be measured in vivo using a severe combined immune deficient (SCID) mouse model. Several mouse models have been developed in which human cells are implanted into immunodeficient mice (collectively referred to as xenograft models); see, for example, Caffan A R, Douglas E, Leuk. Res. 18:513-22, 1994 and Flavell, D J, Hematological Oncology 14:67-82, 1996. As an in vivo xenograft model for psoriasis, human psoriatic skin tissue is implanted into the SCID mouse model, and challenged with an appropriate antagonist. Moreover, other psoriasis animal models in ther art may be used to evaluate the antibodies of the invention, such as human psoriatic skin grafts implanted into AGR129 mouse model, and challenged with an appropriate antagonist (e.g., see, Boyman, O. et al., J. Exp. Med. Online publication #20031482, 2004, incorporated herein by reference). Similarly, tissues or cells derived from human colitis, IBD, arthritis, or other inflammatory lesions can be used in the SCID model to assess the anti-inflammatory properties of the antibody fragments of the invention described herein.

Therapies designed to abolish, retard, or reduce inflammation using antibody fragments of the invention can be tested by administration of such antibodies to SCID mice bearing human inflammatory tissue (e.g., psoriatic lesions and the like), or other models described herein. Efficacy of treatment is measured and statistically evaluated as increased anti-inflammatory effect within the treated population over time using methods well known in the art. Some exemplary methods include, but are not limited to measuring for example, in a psoriasis model, epidermal thickness, the number of inflammatory cells in the upper dermis, and the grades of parakeratosis. Such methods are known in the art and described herein. For example, see Zeigler, M. et al. Lab Invest 81:1253, 2001; Zollner, T. M. et al. J. Clin. Invest. 109:671, 2002; Yamanaka, N. et al. Microbio.l Immunol. 45:507, 2001; Raychaudhuri, S. P. et al. Br. J. Dermatol. 144:931, 2001; Boehncke, W. H et al. Arch. Dermatol. Res. 291:104, 1999; Boehncke, W. H et al. J. Invest. Dermatol. 116:596, 2001; Nickoloff, B. J. et al. Am. J. Pathol. 146:580, 1995; Boehncke, W. H et al. J. Cutan. Pathol. 24:1, 1997; Sugai, J., M. et al. J. Dermatol. Sci. 17:85, 1998; and Villadsen L. S. et al. J. Clin. Invest. 112:1571, 2003. Inflammation may also be monitored over time using well-known methods such as flow cytometry (or PCR) to quantitate the number of inflammatory or lesional cells present in a sample, score (weight loss, diarrhea, rectal bleeding, colon length) for IBD, paw disease score and inflammation score for CIA RA model.

Moreover, psoriasis is a chronic inflammatory skin disease that is associated with hyperplastic epidermal keratinocytes and infiltrating mononuclear cells, including CD4+ memory T cells, neutrophils and macrophages (Christophers, Int. Arch. Allergy Immunol., 110:199, 1996). It is currently believed that environmental antigens play a significant role in initiating and contributing to the pathology of the disease. However, it is the loss of tolerance to self-antigens that is thought to mediate the pathology of psoriasis. Dendritic cells and CD4+ T cells are thought to play an important role in antigen presentation and recognition that mediate the immune response leading to the pathology. We have recently developed a model of psoriasis based on the CD4+CD45RB transfer model (Davenport et al., Internat. Immunopharmacol., 2:653-672). The antibody fragments of the present invention are administered to the mice. Inhibition of disease scores (skin lesions, inflammatory cytokines) indicates the effectiveness of such antibodies in psoriasis.

5. Atopic Dermatitis.

AD is a common chronic inflammatory disease that is characterized by hyperactivated cytokines of the helper T cell subset 2 (Th2). Although the exact etiology of AD is unknown, multiple factors have been implicated, including hyperactive Th2 immune responses, autoimmunity, infection, allergens, and genetic predisposition. Key features of the disease include xerosis (dryness of the skin), pruritus (itchiness of the skin), conjunctivitis, inflammatory skin lesions, Staphylococcus aureus infection, elevated blood eosinophilia, elevation of serum IgE and IgG1, and chronic dermatitis with T cell, mast cell, macrophage and eosinophil infiltration. Colonization or infection with S. aureus has been recognized to exacerbate AD and perpetuate chronicity of this skin disease.

AD is often found in patients with asthma and allergic rhinitis, and is frequently the initial manifestation of allergic disease. About 20% of the population in Western Countries suffers from these allergic diseases, and the incidence of AD in developed countries is rising for unknown reasons. AD typically begins in childhood and can often persist through adolescence into adulthood. Current treatments for AD include topical corticosteroids, oral cyclosporin A, non-corticosteroid immunosuppressants such as tacrolimus (FK506 in ointment form), and interferon-gamma. Despite the variety of treatments for AD, many patients' symptoms do not improve, or they have adverse reactions to medications, requiring the search for other, more effective therapeutic agents.

Pharmaceutical Compositons. For pharmaceutical use, scFc molecule is formulated as a pharmaceutical composition. A pharmaceutical composition comprising an scFc molecule can be formulated according to known methods for preparing pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. In one embodiment, the scFc molecules of the present invention are formulated for parenteral, particularly intravenous or subcutaneous, delivery according to conventional methods. Intravenous administration will be by bolus injection, controlled release, e.g, using mini-pumps or other appropriate technology, or by infusion over a typical period of one to several hours. In general, pharmaceutical formulations will include an scFc molecule in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. When utilizing such a combination therapy, the antibody fragments may be combined in a single formulation or may be administered in separate formulations. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0. 1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. Monospecific antagonists can be individually formulated or provided in a combined formulation. The scFc molecules of the present invention can also be administered in combination with other cytokines such as IL-3,-6 and -I1; stem cell factor; erythropoietin; G-CSF and GM-CSF.

A pharmaceutical composition comprising an scFc molecule is administered to a subject in an effective amount. Generally, the dosage of administered binding molecules of the invention will vary depending upon such factors as the patient's age, weight, height, sex, general medical condition and previous medical history. Typically, it is desirable to provide the recipient with a dosage which is in the range of from about 1 pg/kg to 10 mg/kg (amount of agent/body weight of patient), although a lower or higher dosage also may be administered as circumstances dictate.

Administration of the binding molecules of the invention to a subject can be intravenous, intraarterial, intraperitoneal, intramuscular, subcutaneous, intrapleural, intrathecal, by perfusion through a regional catheter, or by direct intralesional injection. For prevention and treatment purposes, an antagonist may be administered to a subject in a single bolus delivery, via continuous delivery (e.g., continuous transdermal delivery) over an extended time period, or in a repeated administration protocol (e.g., on an hourly, daily, or weekly basis). When administering therapeutic proteins by injection, the administration may be by continuous infusion or by single or multiple boluses. For pharmaceutical use for treatment of neovascular ocular disorders, the scFc molecules are typically formulated for intravitreal injection according to conventional methods.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising binding molecules of the invention can be prepared and inhaled with the aid of dry-powder dispersers, liquid aerosol generators, or nebulizers (e.g., Peffit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer the scFC molecules.

A pharmaceutical composition comprising a scFc molecule of the invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, the scFc molecules of the invention and a pharmaceutically acceptable carrier are administered to a patient in a therapeutically effective amount. A combination of a therapeutic scFc molecule of the present invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response. Effective treatment may be assessed in a variety of ways. In one embodiment, effective treatment is determined by reduced inflammation. In other embodiments, effective treatment is marked by inhibition of inflammation. In still other embodiments, effective therapy is measured by increased well-being of the patient including such signs as weight gain, regained strength, decreased pain, thriving, and subjective indications from the patient of better health.

Determination of effective dosages in this context is typically based on animal model studies followed up by human clinical trials and is guided by determining effective dosages and administration protocols that significantly reduce the occurrence or severity of the subject disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, e.g., to optimize safety and efficacy. Accordingly, a therapeutically or prophylactically effective amount is also one in which any undesired collateral effects are outweighed by beneficial effects of inhibiting angiogenesis. For example, administration of an scFc molecule may have a dosage range from about 0.1 .micro.g to 100 mg/kg or 1 .micro.g/kg to about 50 mg/kg, and more usually 10 .micro.g to 5 mg/kg of the subject's body weight. In more specific embodiments, an effective amount of the agent is between about 1 .micro.g/kg and about 20 mg/kg, between about 10 .micro.g/kg and about 10 mg/kg, or between about 0.1 mg/kg and about 5 mg/kg. Dosages within these ranges can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bimonthly intervals. Alternatively, administrations can be on an irregular basis as indicated by monitoring of a marker such as NK cell activity and/or clinical symptoms of the disease or disorder.

Dosage of the pharmaceutical composition may be varied by the attending clinician to maintain a desired concentration at a target site. For example, if an intravenous mode of delivery is selected, local concentration of the agent in the bloodstream at the target tissue may be between about 1-50 nanomoles of the composition per liter, sometimes between about 1.0 nanomole per liter and 10, 15, or 25 nanomoles per liter depending on the subject's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

A pharmaceutical composition comprising an scFc molecule can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants. (See, e.g., Bremer et al., Pharm. Biotechnol. 10:239, 1997; Ranade, “Implants in Drug Delivery,” in Drug Delivery Systems 95-123 (Ranade and Hollinger, eds., CRC Press 1995); Bremer et al., “Protein Delivery with Infusion Pumps,” in Protein Delivery: Physical Systems 239-254 (Sanders and Hendren, eds., Plenum Press 1997); Yewey et al., “Delivery of Proteins from a Controlled Release Injectable Implant,” in Protein Delivery: Physical Systems 93-117 (Sanders and Hendren, eds., Plenum Press 1997).) Other solid forms include creams, pastes, other topological applications, and the like.

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993), and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 .micro.m to greater than 10 .micro.m. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368 (1985)). After intravenous administration, small liposomes (0.1 to 1.0 .micro.m) are typically taken up by cells of the reticuloendothelial system, located principally in the liver and spleen, whereas liposomes larger than 3.0 .micro.m are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428 (1984)). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133 (1991); Allen et al., Biochim. Biophys. Acta 1150:9 (1993)).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phospatidylcholine, a-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting counter-receptors can be bound to the surface of the liposome, such as antibodies, antibody fragments, carbohydrates, vitamins, and transport proteins. For example, for targeting to the liver, liposomes can be modified with branched type galactosyllipid derivatives to target asialoglycoprotein (galactose) receptors, which are exclusively expressed on the surface of liver cells. (See Kato and Sugiyama, Crit. Rev. Ther. Drug Carrier Syst. 14:287, 1997; Murahashi et al., Biol. Pharm. Bull.20:259, 1997.) In a more general approach to tissue targeting, target cells are prelabeled with biotinylated antibodies specific for a counter-receptor expressed by the target cell. (See Harasym et al., Adv. Drug Deliv. Rev. 32:99, 1998.) After plasma elimination of free antibody, streptavidin-conjugated liposomes are administered. In another approach, targeting antibodies are directly attached to liposomes. (See Harasym et al., supra.)

Polypeptides and antibodies can be encapsulated within liposomes using standard techniques of protein microencapsulation (see, for example, Anderson et al., Infect. Immun. 31:1099 (1981), Anderson et al., Cancer Res. 50:1853 (1990), and Cohen et al., Biochim. Biophys. Acta 1063:95 (1991), Alving et al. “Preparation and Use of Liposomes in Immunological Studies,” in Liposome Technology, 2nd Edition, Vol. III, Gregoriadis (ed.), page 317 (CRC Press 1993), Wassef et al., Meth. Enzymol. 149:124 (1987)). As noted above, therapeutically useful liposomes may contain a variety of components. For example, liposomes may comprise lipid derivatives of poly(ethylene glycol) (Allen et al., Biochim. Biophys. Acta 1150:9 (1993)).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide carriers for intravenous administration of therapeutic proteins (see, for example, Gref et al., Pharm. Biotechnol. 10:167 (1997)).

Other dosage forms can be devised by those skilled in the art, as shown, for example, by Ansel and Popovich, Pharmaceutical Dosage Forms and Drug Delivery Systems, 5th Edition (Lea & Febiger 1990), Gennaro (ed.), Remington's Pharmaceutical Sciences, 19th Edition (Mack Publishing Company 1995), and by Ranade and Hollinger, Drug Delivery Systems (CRC Press 1996).

As an illustration, pharmaceutical compositions may be supplied as a kit comprising a container that comprises a binding molecule or scFc of the invention. The binding molecules of the invention can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise writ en information on indications and usage of the pharmaceutical composition.

A pharmaceutical composition comprising binding molecules of the invention can be furnished in liquid form, in an aerosol, or in solid form. Liquid forms, are illustrated by injectable solutions, aerosols, droplets, topological solutions and oral suspensions. Solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et at., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Other solid forms include creams, pastes, other topological applications, and the like.

The present invention comprises compositions of scFc molecules that are either administered alone as a therapeutic, or are modified to bind to target polypeptides by linking to one or more binding entities, as well as methods for and therapeutic uses of the scFc molecule itself. Such compositions can further comprise a carrier. The carrier can be a conventional organic or inorganic carrier. Examples of carriers include water, buffer solution, alcohol, propylene glycol, macrogol, sesame oil, corn oil, and the like.

The invention is further illustrated by the following non-limiting examples.

### EXAMPLE 1

### Expression of scFc10.1 in CHO

Mammalian Expression Constructs

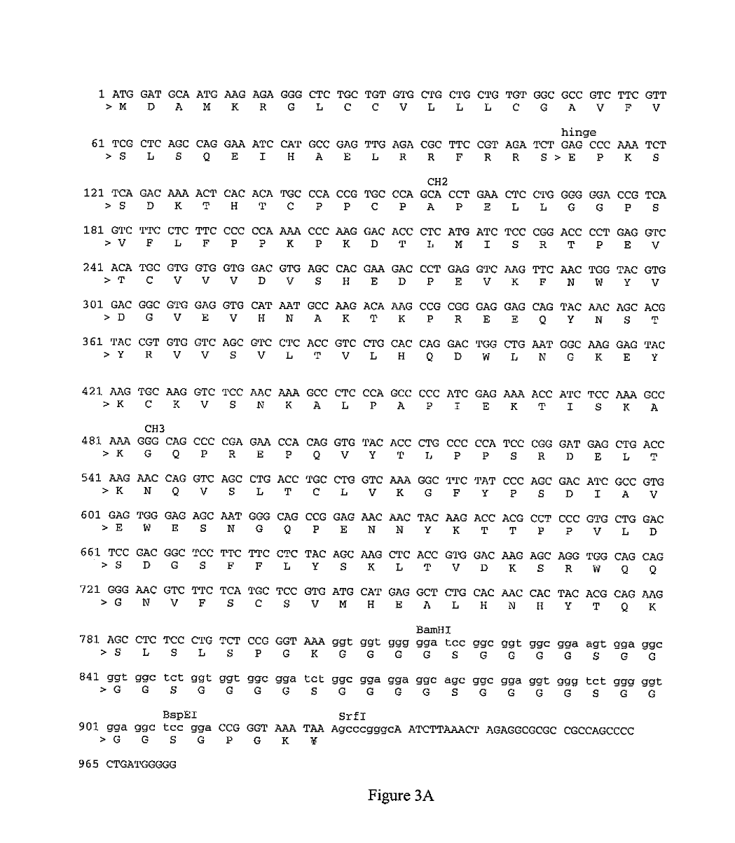

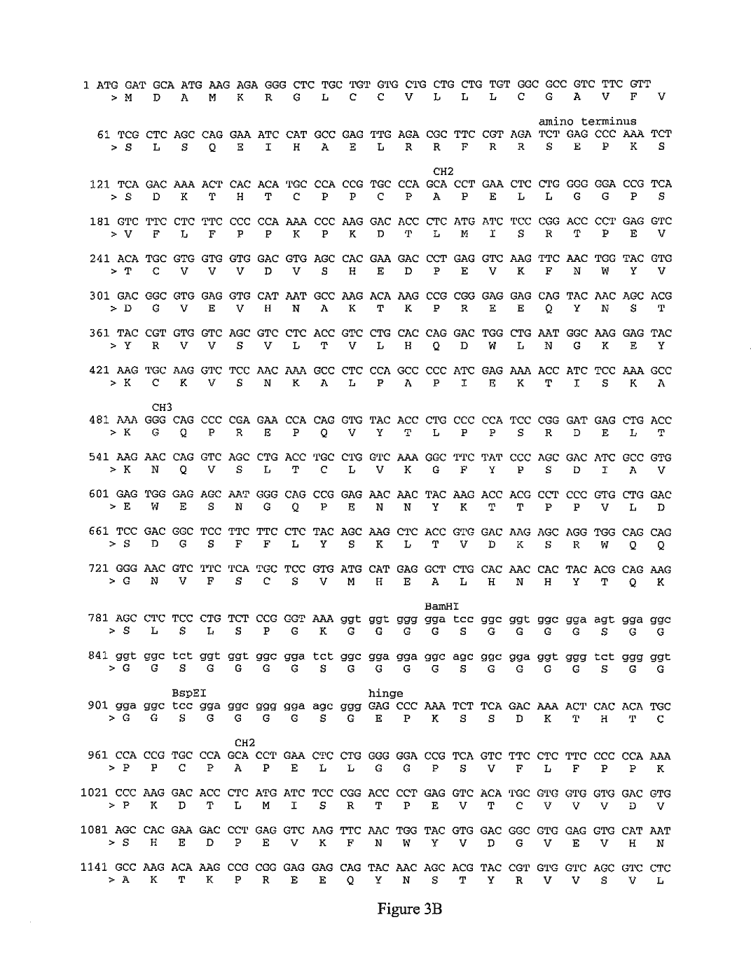

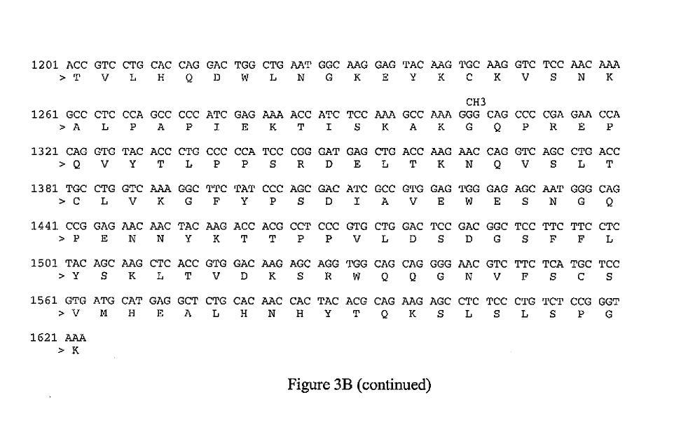

An expression plasmid encoding ScFc10.1 (shown in FIGS. 1A and 3B; SEQ ID NOs: 3 and 4) was constructed via homologous recombination in yeast with two DNA fragments encoding Fc10 (SEQ ID NO:9) connected by a Gly4Ser linker. Specifically, scFc10.1 comprises two intact Fc10 molecules connected by a 41 aa (Gly4Ser)8+Gly linker (SEQ ID NO:11). These linkers are known to be highly flexible, fairly protease resistant and relatively non-immunogenic.

In order to address the complications of cloning two copies of a long cDNA in tandem, the cloning was performed in two stages, first for the intermediate form, an Fc10 cDNA with the Gly4Ser linker and a short polylinker was inserted into mammalian expression vector, pZMP42 and, second, another Fc 10 was inserted by ligation into the short polylinker. Fc 10 consists of residues 216-447 of human immunoglobulin gammal cDNA with C220S mutation (FIG.2). pZMP42 is a derivative of plasmid pZMP21, made by eliminating the hGH polyadenylation site and SV40 promoter/dhfr gene and adding an HCV IRES/dhfr to the primary transcript making it tricistronic. pZMP21 is described in US Patent Application US 2003/0232414 Al, deposited at the American Type Culture Collection, 10801 University Boulevard, Manassas, Va. 20110-2209, designated No.PTA-5266.

The intermediate construct of scFc10.1 cDNA (residues 1-307) (FIG. 3A; SEQ ID NOs: 1 and 2) was constructed using PCR. The upstream primer (SEQ ID NO: 12) for PCR includes from 5′ to 3′ end: 40 bp of flanking sequence from the optimized tPA leader sequence in the vector and 21 bp corresponding to the mature amino terminus from the open reading frame of scFc10. 1. The downstream primer (SEQ ID NO: 13) for the first FcO half of the intermediate scFc10.1 consists from 5′ to 3′ of the bottom strand sequence of 40 bp of (Gly4Ser)4 linker and the last 21 bp of Fc10. The (Gly4Ser)4 linker-short polylinker module was assembled by PCR from three oligonucleotides as shown in SEQ ID NOs:14-16. The two PCR fragments were assembled by overlap PCR using two of the primers above (SEQ ID NOs:12 and 13).

The PCR amplification reaction conditions were as follows: 1 cycle, 94.deg.C., 5 minutes; 25 cycles, 94.deg.C., 1 minute, followed by 65.deg.C., 1 minute, followed by 72.deg.C., 1 minute; 1 cycle, 72.deg.C., 5 minutes. Five .micro.l of each 50 .micro.l PCR reaction was run on a 0.8% LMP agarose gel (Seaplaque GTG) with 1×TAE buffer for analysis. The plasmid pZMP42, which had been cut with BglII, was used for homologous recombination with the PCR fragments. The remaining 45 .micro.l of each PCR reaction and 100 ng of cut pZMP42 were precipitated with the addition of 5 .micro.l 3M Na Acetate and 125 .micro.l of absolute ethanol, rinsed, dried and resuspended in 10 .micro.L water.

One hundred .micro.L of competent yeast cells (S. cerevisiae) were combined with 10 .micro.l of the DNA mixture from above and transferred to a 0.2 cm electroporation cuvette. The yeast/DNA mixtures were electropulsed at 0.75 kV (5 kV/cm), infinity ohms, 25 .micro.F. To each cuveffe was added 600 .micro.l of 1.2 M sorbitol and the yeast was plated in two 300 .micro.l aliquots onto two URA-DS plates and incubated at 30.deg.C. After about 48 hours, approximately 50 .micro.L packed yeast cells were taken from the Ura+ yeast transformants of a single plate, were resuspended in 100 .micro.L of lysis buffer (2% Triton X-100, 1% SDS, 100 mM NaCl, 10 mM Tris, pH 8.0, 1 mM EDTA), 100 .micro.L of Qiagen P1 buffer from a Qiagen miniprep kit (Qiagen, Valencia Calif.) and 20 U of Zymolyase (Zymo Research, Orange, Calif., catalog #1001). This mixture was incubated for 30 minutes at 37.deg.C., and then the remainder of the Qiagen miniprep protocol was performed. The plasmid DNA was eluted in 30 .micro.L water.

Fifty .micro.L electrocompetent E. coli cells (DH12S, Invitrogen, Carlsbad, Calif.) were transformed with 4 .micro.L yeast DNA. The cells were electropulsed at 1.7 kV, 25 .micro.F and 400 ohms. Following electroporation, 600 .micro.l SOC (2% Bacto Tryptone (Difco, Detroit, Mich.), 0.5% yeast extract (Difco), 10 mM NaCl, 2.5 mM KCl, 10 mM MgCl.sub.2, 10 mM MgSO.sub.4, 20 mM glucose) was plated in 120 and 20 .micro.l aliquots on two LB AMP plates (LB broth (Lennox), 1.8% Bacto Agar (Difco), 100 mg/L Ampicillin).

Individual clone inserts were subjected to sequence analysis, and one clone containing the correct sequence for the intermediate construct was selected. This intermediate construct was then used as the base for adding the second Fc10 unit by ligation to make the tandem single chain Fc. The second Fc was made by PCR as described in the previous paragraph using primers to add the flanking sequence with restriction enzyme sites for insertion into the intermediate construct. The upstream primer (SEQ ID NO:17) added Gly4Ser and the BspEI site to the 5′ end of Fc10 (SEQ ID NO:9) and the downstream primer (SEQ ID NO:18) added an SrfI site 3′ of the stop codon. Both the intermediate construct and the modified Fc10 generated by PCR were digested with BspEI and SrfI and purified by agarose gel electrophoresis followed by purification of the isolated band using the Qiagen gel purification kit. The two products, each in 50 .micro.l of elution buffer, were precipitated with the addition of 5 .micro.l of 3M Na Acetate and 125 .micro.l of absolute ethanol, rinsed, dried and resuspended in 10 .micro.L water. 1 ,micro.l of each were combined with 1 .micro.l 5× T4 DNA ligase buffer (Invitrogen, Carlsbad, Calif.), 0.5 .micro.l T4DNA ligase and 1.5 .micro.l water. The reaction was incubated at room temperature for 2 hours and then 1 .micro.l was electroporated into E. coli as described above. Individual clone inserts were subjected to sequence analysis, and one clone containing the correct sequence for the full length scFc10.1 (SEQ ID NOs:3 and 4) was selected. Larger scale plasmid DNA was isolated using the Invitrogen Mega kit (Invitrogen) according to manufacturer's instruction.

Transfection and Selection of scFc10.1 Constructs in CHO Cells

Three sets of 50 .micro.g of the scFc10.1 constructs were separately digested with 100 units of FspI at 37.deg.C. for three hours, precipitated with isopropanol, and centrifuged in a 1.5 mL microfuge tube. The supernatants were decanted off the pellet, and the pellets were washed with 300 .micro.L of 70% ethanol and allowed to incubate for 5 minutes at room temperature. Three tubes were spun in a microfuge for 10 minutes at 14,000 RPM and the supernatants were decanted off the pellet. The pellets were then resuspended in 1 ml of CHO cell tissue culture medium in a sterile environment, allowed to incubate at 60.deg.C. for 30 minutes, and were allowed to cool to room temperature. Approximately 5×10.sup.6 CHO cells were pelleted in each of three tubes and were resuspended using the DNA-medium solution. The DNA/cell mixtures were placed in a 0.4 cm gap cuvette and electroporated using the following parameters: 950 .micro.F, high capacitance, at 300 V. The contents of the cuvettes were then removed, pooled, and diluted to 25 mL with CHO cell tissue culture medium and placed in a 125 mL shake flask. The flask was placed in an incubator on a shaker at 37.deg.C., 6% CO.sub.2 with shaking at 120 RPM.

The CHO cells were subjected to nutrient selection followed by step amplification to 100 nM methotrexate (MTX), 250 nM MTX, and then to 500 nM MTX. Tagged protein expression was confirmed by Western blot, and the CHO cell pool was scaled-up for harvests for protein purification.

### EXAMPLE 2

### Expression of scFc10.2 in CHO

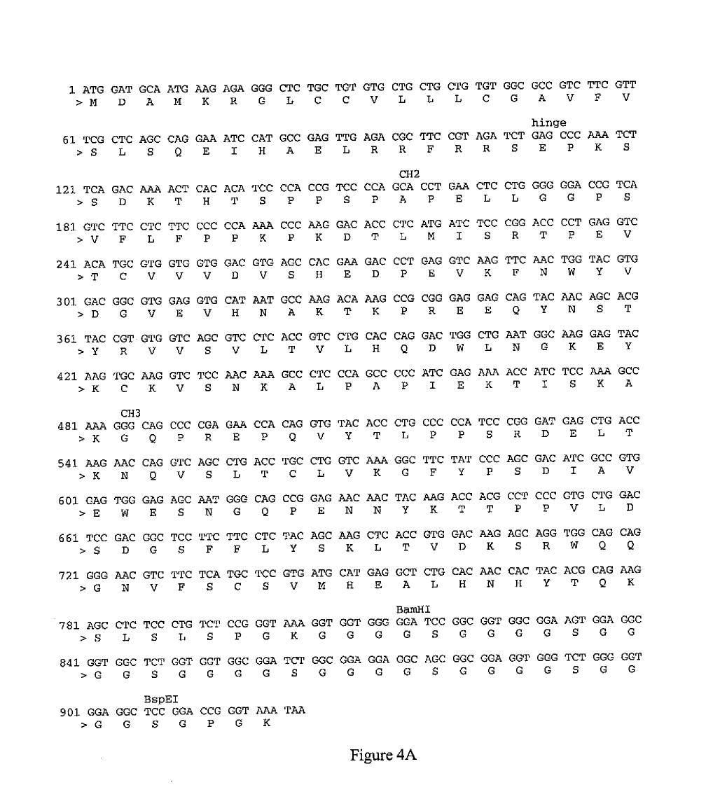

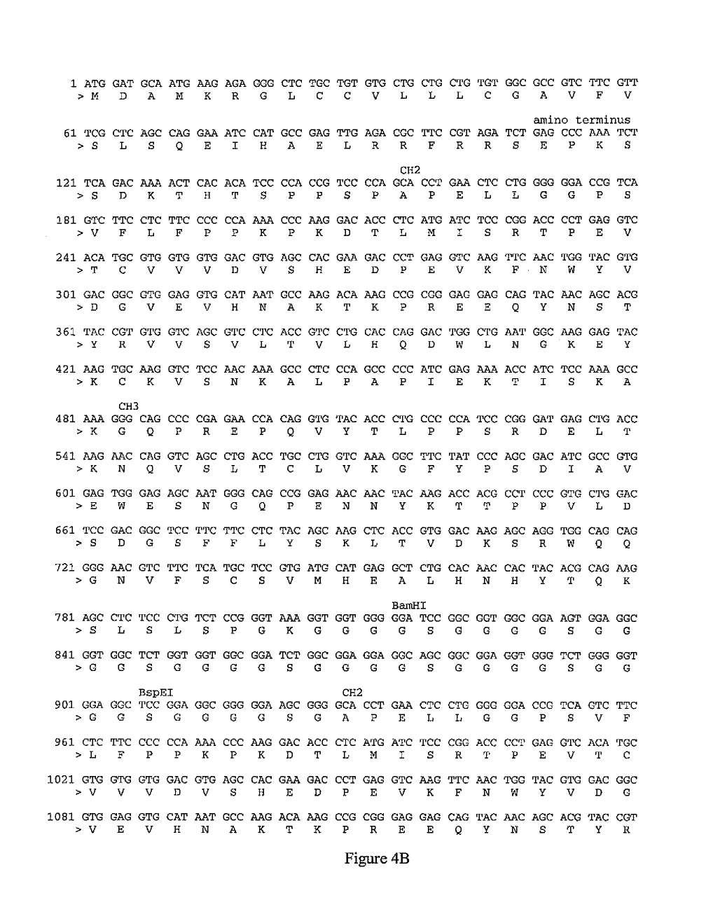

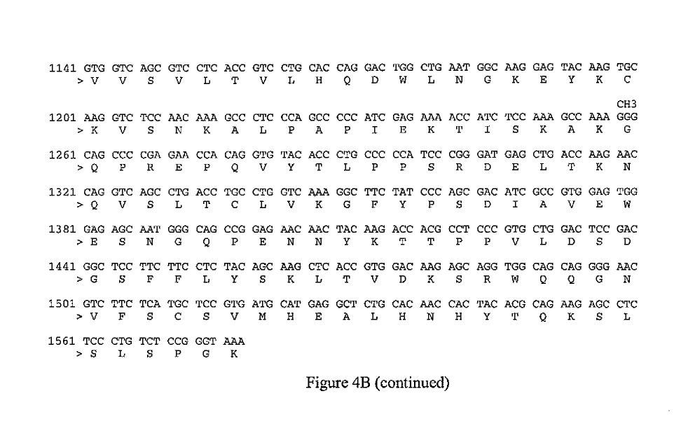

An expression plasmid encoding scFc10.2 (shown in FIGS. 1C and 4B; SEQ ID NOs:21 and 22) was constructed via homologous recombination in yeast with two DNA fragments encoding Fc10 (SEQ ID NO:9) connected by a Gly4Ser linker. This construct differs from scFc10.1 (as described in Example 1) in that the first Fc unit has two mutations in the hinge, substituting serines for the two cysteines, C226S and C229S, and removing the hinge entirely from the second Fc unit. The hinge is known to be important in effector function so the omission of this region is expected to alter the functionality of this form of the Fc molecule. As before, in order to address the complications of cloning two copies of a long cDNA in tandem, the cloning was performed in two stages, first for the intermediate construct, an Fc10 cDNA with the two mutations upstream, the Gly4Ser linker and a short polylinker downstream was inserted into mammalian expression vector, pZMP42 and second another Fc10 was inserted by ligation into the short polylinker. Fc10 and the vector are the same as described previously for scFc10.

The intermediate construct of scFc10.2 cDNA (residues 1-307) (FIG. 4A; SEQ ID NOs: 19 and 20) was constructed using PCR. There were two upstream primers (SEQ ID NOs: 23 and 24) for PCR to code for the two mutations and the flanking sequence, including from 5′ to 3′ end: 40 bp of flanking sequence from the optimized tPA leader sequence in the vector and 21 bp corresponding to the mature amino terminus from the open reading frame of scFc10.2, and the next primer consisted of 52 bp from the hinge sequence with C226S and C229S. The downstream primer (SEQ ID NO:25) for the first Fc10 half of the intermediate scFc10.2 consists from 5′ to 3′ of the bottom strand sequence of 40 bp of (Gly4Ser)4 linker and the last 21 bp of Fc10. The (Gly4Ser)41inker-short polylinker module was assembled by PCR from three oligonucleotides (SEQ ID NOs:14-16), the same set as for scFcIO.I. The two PCR fragments were assembled by overlap PCR using two of the primers above (SEQ ID NOs: 23 and 24), as for scFc10.1.

PCR reaction conditions, purification of DNA products, transformation of yeast and E. coli, identification and sequencing of clones were all performed as described for scFc10.1 in Example 1 above.

The intermediate construct for scFc10.2 (SEQ ID NOs:19 and 20)was then used as the base for adding the second Fc10 unit by ligation to make the tandem single chain Fc. The second Fc was made by PCR as described in the previous paragraph using primers to add the flanking sequence with restriction enzyme sites for insertion into the intermediate construct. The upstream primer (SEQ ID NO:26) added Gly4Ser and the BspEI site to the 5′ end of Fc10 and the downstream primer (SEQ ID NO:27) added an SrfI site 3′ of the stop codon. Both the intermediate construct and the modified Fc10 generated by PCR were digested with BspEI and SrfI and purified by agarose gel electrophoresis followed by purification of the isolated band using the Qiagen gel purification kit. The two products, each in 50 .micro.l of elution buffer, were precipitated with the addition of 5 .micro.l of 3M Na Acetate and 125 .micro.l of absolute ethanol, rinsed, dried and resuspended in 10 .micro.L of water. 1 .micro.l of each were combined with 1 .micro.l of 5× T4 DNA ligase buffer (Invitrogen, Carlsbad, Calif.), 0.5 .micro.l T4DNA ligase and 1.5 .micro.l water. The reaction was incubated at room temperature for 2 hours and then 1 .micro.l was electroporated into E. coli as described above. Individual clone inserts were subjected to sequence analysis, and one clone containing the correct full length scFc10.2 sequence (SEQ ID NOs: 21 and 22) was selected.

Larger scale plasmid DNA was isolated using the Invitrogen Mega kit (Invitrogen) according to manufacturer's instruction. Transfection, selection, characterization and scale up of the scFc10.2 construct in CHO cells was carried out as described for the scFc10.1 construct previously in Example 1 above.

### EXAMPLE 3

### Expression of scFc10.3 in CHO

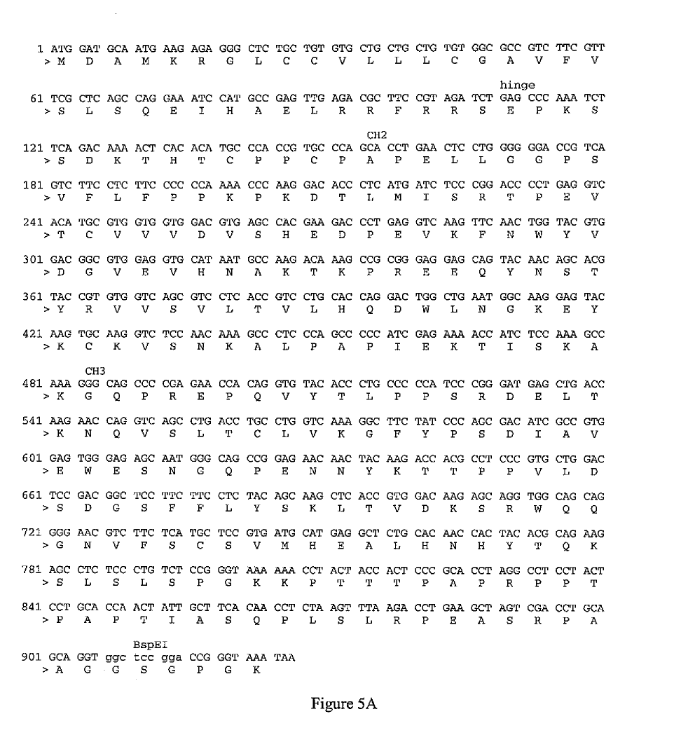

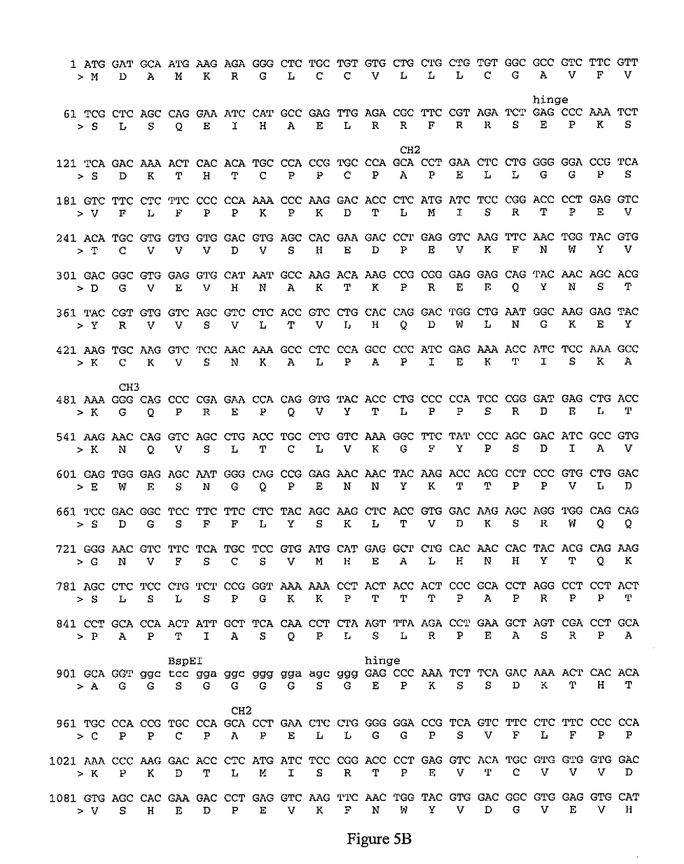

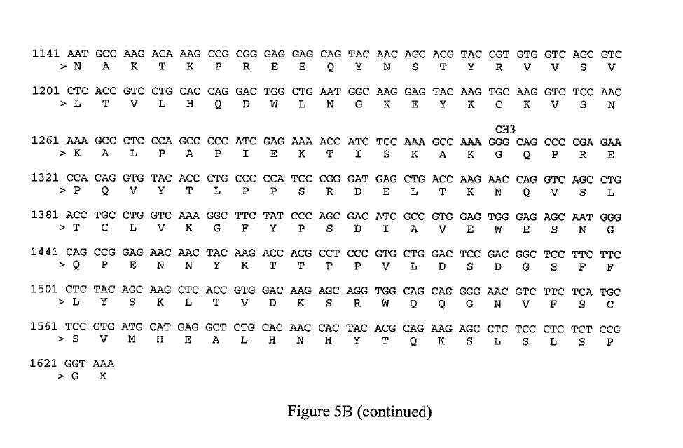

An expression plasmid encoding scFc10.3 (shown in FIGS. 1C and 5B; SEQ ID NOs:30 and 31) was constructed via homologous recombination in yeast with two DNA fragments encoding Fc10 (SEQ ID NO:9) connected by a Gly4Ser linker. This construct differs from scFc10.1 in Example 1 in that the two Fc monomers are connected by a section of the stalk region of human CD8 alpha chain (SEQ ID NOs:32 and 33). The CD8 stalk is heavily O-glycosylated and structural analysis indicates that it is an extended structure. As before, in order to address the complications of cloning two copies of a long cDNA in tandem, the cloning was performed in two stages: first for an intermediate construct (SEQ ID NOs:28 and 29), an Fc10 cDNA with the CD8 stalk and a short polylinker downstream was inserted into mammalian expression vector, pZMP42; and second another Fc10 was inserted by ligation into the short polylinker. Fc10 and the vector are the same as described previously for scFc10.1 in Example 1 above.

The intermediate construct of scFc10.3 cDNA (residues 1-308) (FIG. 5A; SEQ ID NOs: 28 and 29) was constructed using PCR. The upstream primer was the same as for scFc10.1 (SEQ ID NO:12). The downstream primer (SEQ ID NO:34) for the Fc10 part of the intermediate scFc10.3 consists from 5′ to 3′ of the bottom strand sequence of 40 bp of CD8 alpha stalk linker and the last 21 bp of Fc10. The CD8 linker-short polylinker module was assembled by PCR from three oligonucleotides (SEQ ID NOs: 15, 35 and 36). The two PCR fragments were assembled by overlap PCR using two of the primers above (SEQ ID NOs: 12 and 16), as for scFc10.1.

PCR reaction conditions, purification of DNA products, transformation of yeast and E. coli, identification and sequencing of clones were all performed as described for scFc10.1,

A clone with the expected sequence (SEQ ID NOs:28 and 29) was identified for further use.

The intermediate construct for scFc10.3 was then used as the base for adding the second Fc10 unit by ligation to make the tandem single chain Fc (SEQ ID NOs:30 and 31). The second Fc was the same fragment as that described for scFc10.1 in the previous example, cloned into the scFc10.3 intermediate as described for scFc10.1.

Larger scale plasmid DNA was isolated using the Invitrogen Mega kit (Invitrogen) according to manufacturer's instruction. Transfection, selection, characterization and scale up of the scFc10.3 construct in CHO cells was carried out as described for the scFc10.1 construct previously.

### EXAMPLE 4

### Purification of the scFc Molecules

One liter of conditioned media from CHO DXB II cells expressing scFc10.1 (SEQ ID NOs:3 and 4), scFc10.2 (SEQ ID NOs:21 and 22), or scFc10.3 (SEQ ID NOs:30 and 31) was sterile-filtered through 0.22 .micro.m filter. A five mL column of Poros A50 resin (AB Biosystems) was prepared and equilibrated in 1.61 mM citric acid, 2.4 mM dibasic sodium phosphate, 150 mM NaCl at pH 7.0 and the media loaded over the column at 4.deg.C. Once the load was complete, the column washed with 10 column volumes of equilibration buffer, monitoring the absorbance at A280 nm. Once the baseline was stable for one column volume, elution of bound protein was accomplished via a gradual pH shift (10 column volumes) to elution buffer (19.9 mM citric acid, 5.1 mM dibasic sodium phosphate, 150 mM NaCl at pH 3.0). Fractions were immediately neutralized by collection into 2M Tris at pH 8.0. Fractions were analyzed by RP-HPLC and SDS-PAGE and pooled based upon the presence of single chain Fc. Yields: scFc10.1 yielded 8.7 mg; scFc10.2 yielded 3 mg; and scFc10.3 yielded 6.7 mg.

### EXAMPLE 5

### The scFc Molecules Have an Inhibitory Effect in Immune Complex Assays

These scFcs described in Examples 1-3 above were tested in two assays for inhibitory activity in immune complex assays.

Immune Complex Precipitation Methods: Chicken egg ovalbumin (OVA) was dissolved to a final concentration of 15.0 .micro.g/mL in phosphate buffered saline (PBS) and combined with 300 .micro.g rabbit polyclonal anti-OVA antibodies/mL in a final volume of 200 .micro.L in the presence and absence of the indicated concentration of the single chain Fc molecule. Immediately thereafter, turbidity of the reaction mixture was monitored at 350 nm every 30 seconds for 5-10 min at 37.deg.C. with the aid of a spectrophotometer. Linear regression was used to calculate the slope of the linear portion of the turbidity curves and the pFCGR-mediated inhibition of immune complex precipitation was expressed relative to incubations containing anti-OVA and OVA alone.

Cytokine Secretion from Mast Cells: Immune complexes were prepared by mixing 300 .micro.L of rabbit polyclonal anti-OVA with 75.0 .micro.L of 1 mg OVA/mL in PBS in a final volume of 5.0 mL of PBS. After incubation at 37.deg.C. for 30-60 minutes, the mixture was placed at 4.deg.C. for 18-20 hours. The immune complexes were collected by centrifugation at 12,000 rpm for 5.0 min, the supernatant fraction was removed and discarded, and the immune complex precipitate was resuspended 1.0 mL of ice cold PBS. After another wash, the immune complexes were resuspended in a final volume of 1.0 mL ice cold PBS. Protein concentration was determined using the BCA assay.

MC/9 cells were sub-cultured in Medium A (DMEM containing 10% fetal bovine serum, 50.0 .micro.M .beta.-mercaptoethanol, 0.1 mM non-essential amino acids, 1.0 mM sodium pyruvate, 36.0 .micro.g/mL L-asparagine, 1.0 ng/mL recombinant mL-3, 5.0 ng/mL recombinant mL-4, 25.0 ng/mL recombinant mSCF) to a density of 0.5-3×10.sup.6 cells/mL. Cells were collected by centrifugation at 1500 rpm for 5.0 min and the cell pellet was washed in Medium A (without cytokines) and resuspended in Medium A at 2.0×10.sup.6 cells/mL. Aliquots of cells (2.0×10.sup.5 cells) were incubated with 10.0 .micro.g/well of OVA/anti-OVA immune complexes (IC's) in a final volume of 200 .micro.L of Buffer A in a 96-well microtiter plate in the presence and absence of the indicated concentration of single chain Fc. After 4.0 h at 37.deg.C., the media was removed and centrifuged at 1500 rpm for 5.0 min. The cell-free supernatant fractions were collected and aliquots were analyzed for the presence of IL-6, IL-13, TNF.alpha., and MCP-1 cytokine release using a Luminex cytokine assay kit.

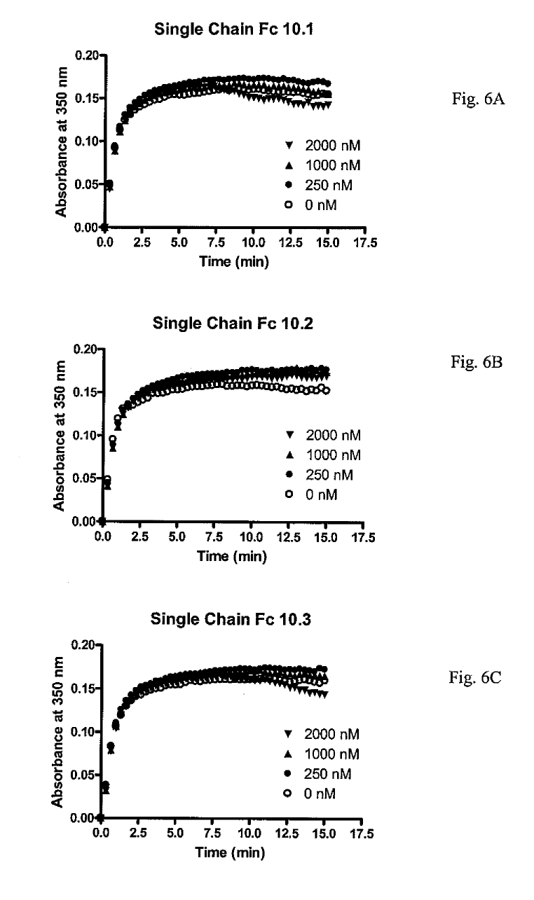

Results: To evaluate whether scFc polypeptides (e.g., scFc10.1, scFc10.2 and scFc10.3) could block immune complex precipitation, an anti-OVA/OVA immune complex precipitation assay was established based on the methods of MOller NPH (1979) Immunology 38: 631-640 and Gavin A L et al., (1995) Clin Exp Immunol 102: 620-625. Incubation of anti-OVA and OVA at 37.deg.C. produced a time-dependent increase in optical density of the solution mixture (FIG. 6), an observation consistent with the formation of insoluble anti-OVA/OVA immune complexes. The addition of single chain Fc 10.1 (FIG. 6A) 10.2 (FIG. 6B), or 10.3 (FIG. 6C) at the start of the assay did not produced any effects on immune complex precipitation over the range of concentrations used (0-2000 nM). The addition of a recombinant soluble version of human CD64, in contrast, blocked the precipitation of the OVA/anti-OVA immune complexes. Since the precipitation of antigen:antibody immune complexes appears to be dependent on non-covalent interactions between the antibody Fc heavy chains (MOller NPH (1979) Immunology 38: 631-640) these data suggest that the single chain Fc molecules do not bind to the Fc portion of the anti-OVA antibodies.

Mast cells are thought to mediate immune complex-mediated inflammation in a variety of immune disorders such as type III hypersensitivity reactions (Ravetch J V (2002) J Clin Invest 110, 1759-1761; Sylvestre D L and Ravetch J V (1996) Immunity 5, 387-390; Jancar S and Crespo M S (2005) Trends Immunology 26, 48-55). Binding of immune complexes to mast cell Fc.gamma. receptors is thought to induce the secretion of pro-inflammatory cytokines, such as IL-6 and TNF.alpha. (Ravetch J V (2002) J Clin Invest 110, 1759-1761; Jancar S and Crespo M S (2005) Trends Immunology 26, 48-55), which subsequently leads to neutrophil infiltration and tissue damage. To evaluate whether cytokine secretion from mast cells could be stimulated by immune complexes, the murine mast cell line MC/9 was incubated in the presence and absence of preformed rabbit anti-OVA/OVA immune complexes. Incubation with anti-OVA/OVA immune complexes produced a time and concentration dependent increase in the accumulation of the inflammatory cytokines IL-6, IL-13, TNF.alpha., and MCP-1 within the MC/9 cell conditioned media. Cytokine production was not altered, in contrast, when MC/9 cells were incubated with an equivalent concentration of rabbit anti-OVA IgG alone. These data demonstrate that MC/9 cells respond to immune complexes by the production of inflammatory cytokines.

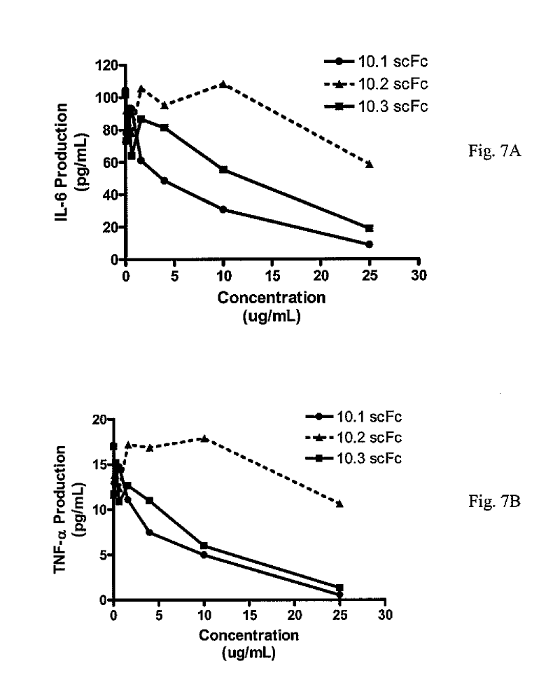

Incubation of MC/9 cells with anti-OVA/OVA immune complexes in the presence of increasing amounts of single chain Fc10.1 produced dose-dependent reductions in the accumulation of IL-6 (FIG. 7A) and TNF.alpha. (FIG. 7B). A similar reduction in the accumulation of IL-13 and MCP-1 by single chain Fc 10.1 was also observed. Single chain Fc 10.3 was less potent at blocking immune complex-mediated cytokine secretion than single chain Fc 10.1 while single chain Fc 10.2 showed little or no inhibition of IL-6 and TNF.alpha. secretion (FIG. 7). Similarly, single chain Fc 10.2 had no effect on IL-13 and MCP-1 accumulation in mast cell conditioned media, while single chain Fc 10.3 was less potent than single chain Fc 10.1. These data demonstrate that single chain Fc 10.1 and to a lesser extent 10.3 can block the binding and signaling of immune complexes in mouse mast cells. These data suggest that the single chain Fc 10.1 and 10.3 bound to cell surface Fc receptors and blocked their interaction with extracellular immune complexes, thus preventing cytokine release.

None of these molecules interacted directly with immune complexes but scFc10.1 (SEQ ID NO:4) and scFc10.3 (SEQ ID NO:31) did interfere with the interaction of immune complexes and mast cells, implying that there is an interaction with Fc receptors on mast cells. Specifically, scFc10.1 competitively blocked immune complex mediated secretion of IL-6, TNF-alpha, MCP-1, and IL-13 from murine MC/9 mast cells (scFc10.3 also showed some inhibitory activity, but was less active than scFc10.1). In contrast, the scFc10.2, containing the mutated hinge region described in Example 2, had little or no activity. These data suggested that the scFc10.1 bound to cell surface Fc receptors and blocked their interaction with extracellular immune complexes, thus preventing cytokine release.

Additionally, the scFc of the invention (namely, scFc10.1, scFc10.2 and scFc10.3) do not affect immune complex precipitation. Neither scFc10.1, scFc10.2, nor scFc 10.3 produced any significant effects on the in vitro precipitation of OVA/anti-OVA immune complexes. These data suggested that these scFc do not interact with either the OVA or anti-OVA antibodies. The inhibition of cytokine secretion described above is thus likely due to blockade of cell surface Fc gamma receptors. Accordingly, the scFc molecules of the invention can act alone as a therapeutic to treat immune diseases or may be used as a fusion partner with one or more target-specific binding entities, such as scFv molecules or tandem pairs of scFv molecules to form potent multispecific antibody fragment drug candidates

### EXAMPLE 6

### Stimulation of NK cells with the scFc Molecules and Binding of the Same to Human NK Cells

Two wells each of a 24-well flat bottom tissue culture plate were coated with 10 .micro.g/ml of scFc10.1 SEQ ID NO:4), scFc10.2 (SEQ ID NO:22), scFc10.3 (SEQ ID NO:31), Human Fc10 (SEQ ID NO:10) or HuIgG (Calbiochem, San Diego, Calif.) diluted into phosphate buffered saline, and incubated at 4.deg.C. overnight to coat plates. Following overnight incubation, plates were washed one time with PBS and then one time with RPMI 1640 prior to plating cells. Human NK cells were isolated from whole peripheral blood mononuclear cells using the NK Cell Isolation Kit II and protocol (Miltenyi Biotec #130-091-152, Auburn, Calif.). Freshly isolated NK cells were then added to the coated plates at 1×10.sup.6 cells per milliliter in RPMI Complete (RPMI 1640 supplemented with 10% Hu AB Serum, 1 mM Sodium Pyruvate, 2 mM L-Glutamine, 10 mM HEPES, and 50 .micro.M beta.-mercaptoethanol (Invitrogen, Carsbad, Calif.).) Human IL-21 (SEQ ID NO:61) was added to one of each of the duplicate coated wells to a final concentration of 20 ng/ml. NK cells were then incubated for 4 days at 37.deg.C., 5% CO.sub.2. Plates were then spun and 0.5 mL of each supernatant transferred to eppendorf tubes and frozen at −20.deg.C. until analysis. The levels of Human IFN-.gamma. were determined using a Beadmate Human IFN-.gamma. kit (Upstate #46-131, Temecula, Calif.) and Bio-Plex 200 Instrument (Biorad, Hercules, Calif.). Data was then transferred into Excel (Microsoft, Redmond, Wash.) for further analysis and graphing.

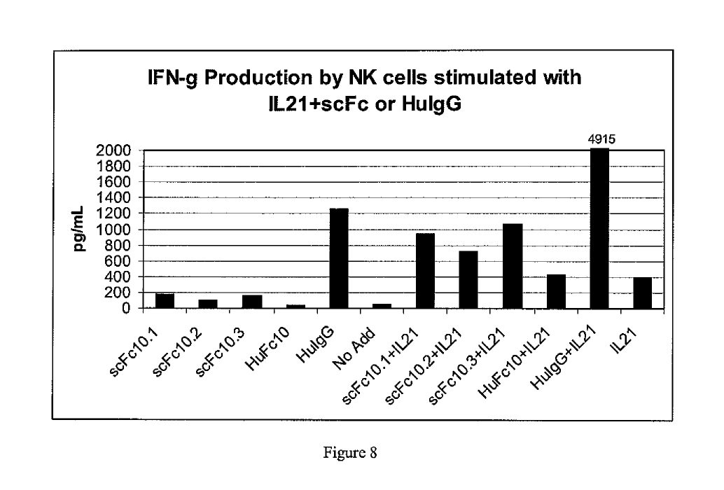

Results: Co-stimulation of human NK cells with IL-21 and plate-bound Human IgG causes a synergistic increase in IFN-.gamma. production by these cells. In order to test whether the scFc molecules of the invention were able to co-stimulate NK cells in this context, human NK cells were stimulated with plate-bound scFc in the presence of IL-21. In this experiment, human NK cells stimulated with human IL-21 in combination with scFc10.1, scFc10.2, or scFc10.3 produced 2-3 times more IFN-.gamma. than NK cells stimulated with IL-21 alone (FIG. 8). These results indicate that the scFc molecules of the invention are able to co-stimulate NK cells via Fc receptors on the surface of these cells.

Staining of Human NK cells with scFc-biotin: Human NK cells were isolated from peripheral blood as described previously. Three different scFc constructs (scFc10.1, scFc10.2, and scFc10.3) as well as control HuFc10 proteins were biotinylated using the Sulfo-NHS-LC-Biotin Ezlink kit and protocol (#21335 Pierce, Rockford, Ill.). For staining, freshly isolated NK cells were washed one time with facs wash buffer (FWB: Hanks Buffered Salt Solution+2% normal goat serum+2% bovine serum albumen+0.02% sodium azide). NK cells were then plated into a 96-well round bottom plate at a concentration of 2×10.sup.5 cells per well. Cells were spun down at 1200 rpm and then resuspended in 5 .micro.g/ml biotinylated scFc or HuFc10 in 50 .micro.L. Control wells were also included with NK cells pre-blocked with unlabeled scFc or HuFc10 at a concentration of 50.micro.g/ml. Cells were then incubated for 30 minutes at 4.deg.C. and then washed twice with 200.micro.L FWB. Following washes, cells were resuspended in 50 .micro.L of phycoerythrin-labeled streptavidin diluted into FWB at 1:200 (Jackson Immunoresearch #016-084-110 West Grove, Pa.). Cells were incubated for 30 minutes at 4.deg.C., washed twice with FWB and then resuspended in 200.micro.L FWB following final wash. Cells were immediately collected and analyzed on a Facscalibur flow cytometer using Cellquest software. (BD Biosciences).

Results: Human NK cells bind scFc10.1 and scFc10.3 and this staining is partially blockable with a 10-fold excess of unlabeled protein. The scFc10.2 appears to stain NK cells weakly and this staining is also partially blockable with unlabeled protein. These results indicate that Fc receptors on the surface of human NK cells are able to recognize and bind scFc10.1, scFc10.2, and cFc10.3.

### EXAMPLE 7

### scFc Molecules and scFv Fusions bind FcgammaR1A and FCRN

A. FcgammaR1A: The ability of scFc10.1 (SEQ ID NO:4), scFc10.2 (SEQ ID NO:22), scFc10.3 (SEQ ID NO:31), and each fused with an scFv Herceptin binding entity (SEQ ID NOs:60, 48 and 64, respectively) to bind to FcgammaR1a was assessed using a direct ELISA. In this assay, wells of 96 well polystyrene ELISA plates were first coated with 50 .micro.L/well of the extracellular domain of an FcgammaR1A (FcgR1a. See, e.g. GenBank Accession No.: P12314.2; GI:50403717) at 500 ng/mL in Coating Buffer (0.1M Na.sub.2CO.sub.3, pH 9.6). Plates were incubated overnight at 4.deg.C. after which unbound protein was aspirated and the plates washed twice with 300 .micro.L/well of Wash Buffer (PBS-Tween defined as 0.137M NaCl, 0.0027M KCl, 0.0072M Na.sub.2HPO.sub.4, 0.0015M KH.sub.2PO.sub.4, 0.05% v/v polysorbate 20, pH 7.2). Wells were blocked with two sets of 100 .micro.L/well of SuperBlock (Pierce, Rockford, Ill.) for a minimum of 5 minutes each, after each set the plate was poured out to empty. Serial 10-fold dilutions in Blocking Buffer (PBS-Tween plus 1% w/v bovine serum albumin (BSA)) of the purified protein were prepared beginning with an initial dilution of 500 ng/mL and ranged to 0.5 ng/mL. Triplicate samples of each dilution were then transferred to the assay plate, 50 .micro.L/well, in order to bind specific scFc protein to the assay plate. An scFv mouse anti-human PDGFR.beta. -Fc5 served as a negative control (SEQ ID NOs:71 and 72). Fc5 is a mutated IgG1 Fc and is effector function is negative. Commercial Herceptin (Dubin Medical, San Diego, Calif.), was added as a positive assay control. Following a 2-hour incubation at 37.deg.C. with agitation, the wells were aspirated and the plates washed twice as described above. Horseradish peroxidase labeled goat anti-human IgG, Fc specific antibody (Jackson ImmunoResearch, West Grove, Pa.) at a concentration of 1:2000 was then added to the wells, 50 .micro.L/well. Following a 1-hour incubation at 37.deg.C. with agitation, unbound antibody was aspirated from the wells and the plates washed five times with 500 .micro.L/well of Wash Buffer. Tetra methyl benzidine (TMB) (BioFX Laboratories, Owings Mills, Md.), 50 .micro.L/well, was added to each well and the plates incubated for 5 minutes at room temperature. Color development was stopped by the addition of 50 .micro.L/well of 450 nm TMB Stop Reagent (BioFX Laboratories, Owings Mills, Md.) and the absorbance values of the wells read on a Bio-Tex EL808 instrument at 450 nm.

Conclusion: The direct ELISA assay indicate that scFc10.1, scFc10.2, scFc10.3 alone and the Herceptin scFv fusions bound to FcgammaR1A to similar levels as the positive control Herceptin whole immunoglobulin.

B. FCRN binding assay for measuring binding of Herceptin-scFv-scFc10.1 and Herceptin-scFv-Fc10 to FCRN at pH 6.0.

Materials and Methods: Day 1: A Nunc Maxisorp 96 well elisa plate (cat #44-2404) was coated with 300 ng/well NeutrAvidin (Pierce Chemical Co. cat. #31000) made up in 100 mM NaHCO.sub.3, pH 9.3. Plate was incubated at 4.deg.C. overnight. Day 2: The plate was washed 5 times with 0.1% Tween-20/PBS (PBST). The plate was then blocked with 250.micro.1/well of blocking buffer containing 0.8% NaCl, 0.02% KCL, 0.102% Na.sub.2HPO.sub.4, 0.02% KH.sub.2PO.sub.4, 1% BSA, 0.05% Polysorbate, 0.05% Proclin 300 pH 7.2, for one hour at room temperature. The plate was then washed 2 times with PBST. Each well was then coated with 150 ng of biotinylated FCRN protein (See, e.g., GenBank Accession No.: P55899.1 GI:2497331) diluted in PBST+1% BSA. The plate was incubated at room temperature for one hour. Herceptin fusion proteins (Herceptin-scFv-scFc10.1 (SEQ ID NO:48) and Herceptin-scFv-Fc10(SEQ ID NO:60)) and control antibodies (Herceptin, Dublin Medical, San Diego, Calif., for example) were diluted in 100 mM NaPO.sub.4, 0.05% Tween 20 (v/v), +0. 1% BSA adjusted to pH 6.0 (pH 6.0 buffer) at concentrations ranging from 150 mM to 0.0185 mM. Samples were tested in duplicate at a volume of 50.micro.1/well of each concentration. A pH 6.0 buffer only was run as a control to determine the background levels on each plate. The plate was incubated at room temperature for two hours. After the binding step, the plate was washed with 250 .micro.l/well of pH 6.0 buffer. The plate was incubated in wash buffer at room temperature for a total of one hour with a wash step performed every twenty minutes. Following the wash steps, the bound antibody was detected with 100 .micro.l/well of HRP goat anti-human IgG F(ab)2 fragment FC gamma specific secondary antibody (Jackson Immunoresearch Cat. #109-036-098). The secondary antibody was diluted 1:5,000 in the pH 6.0 buffer, and the incubation was done for one hour at room temperature. The plate was then washed 5 times with PBST. Finally, 100 .micro.l of TMB (TMBW-1000-01, BioFX Laboratories) was added to each well, and the plate was developed at room temperature for approximately three minutes. At this point, 100 .micro.l/well of stop buffer (STPR-100-01, BioFX Laboratories) was added to quench the reaction. The plate was read on a spectrophotometer at a wave length of 450/570nm. OD values were examined to compare binding patterns at pH 6.0 of the various constructs. Figure

Results: All three molecules tested (SEQ ID NO:48, SEQ ID NO:60 and control antibody Herceptin) showed similar binding to FcRn at pH6.0 indicating that the monovalent scFc molecules retain antibody binding properties significant for enhanced half-life in vivo when compared to the bivalent molecules.

### EXAMPLE 8

### Construction of a scFc with a Single Chain Fv that Binds the HER2/c-erb-2 Gene Product

The scFc molecules of the present invention were cloned into two expression vectors, pZMP3 1-Puro and pZMP42.

A) pZMP3 1-Puro Expression Vector:

1) Construction of cDNA in Vector:

Plasmid pZMP3 1-Puro is a mammalian expression vector containing an expression cassette having the chimeric CMV enhancer/MPSV promoter, an EcoRT site for linearization prior to yeast recombination, an internal ribosome entry element from poliovirus, an E. coli origin of replication; a mammalian selectable marker expression unit comprising an SV40 promoter, enhancer and origin of replication, a Puromycin gene, and the SV40 terminator; and URA3 and CEN-ARS sequences required for selection and replication in S. cerevisiae.

An expression construct containing a scFc with a HER2/c-erb-2-binding scFv (wild-type Fc of IgG1), with a 25 mer Gly-Ser linker linking the variable heavy and light chains of the scFv, and a 5 mer Gly-Ser linker linking the scFv and Fc region was constructed via a four step-PCR and homologous recombination using a DNA fragment encoding the HER2/c-erb-2-binding scFv-Fc and the expression vector pZMP3 1-Puro. The cDNA sequence of the HER2/c-erb-2-binding scFv-scFc MCV14 construct is shown in SEQ ID NO:47. The encoded polypeptide has the amino acid sequence shown in SEQ ID NO:48.

The PCR fragment encoding HER2/c-erb-2-binding scFv-scFc was constructed to contain a 5′ overlap with the pZMP31-Puro vector sequence in the 5′ non-translated region, the HER2/c-erb-2-binding scFv coding region (nucleotides 58-813), the Fc coding sequence (nucleotides 829-1527), and a 3′ overlap with the pZMP31-Puro vector in the poliovirus internal ribosome entry site region. The signal sequence was the murine 26-10 VL signal sequence (nucleotides 1-57). The first PCR amplification reaction used the 5′ oligonucleotide “zc56623” (SEQ ID NO:39) and the 3′ oligonucleotide “zc56624” (SEQ ID NO: 40). The second PCR amplification reaction used the 5′ oligonucleotide “zc56609” (SEQ ID NO:41), and the 3′ oligonucleotide “zc56610” (SEQ ID NO:42), and a previously generated DNA clone of the HER2/c-erb-2-binding scFv as the template (SEQ ID NO:43).

The third PCR amplification reaction used the 5′ oligonucleotide “zc56614” (SEQ ID NO: 45), and the 3′ oligonucleotide “zc56625” (SEQ ID NO:46), and a previously generated DNA clone of the wild-type human Fc from IgG1 as the template. The fourth PCR amplification reaction used the 5′ oligonucleotide “zc56623” (SEQ ID NO:39), and the 3′ oligonucleotide “zc56625” (SEQ ID NO:46), and the first three previously generated PCR templates in an overlap PCR reaction.

The PCR amplification reaction conditions were as follows: 1 cycle, 95 .deg.C., 2 minutes; 30 cycles, 95 .deg.C., 15 seconds, followed by 55 .deg.C., 30 seconds, followed by 68 .deg.C., 1 minute 45 seconds. The PCR reaction mixture was run on a 1% agarose gel and the DNA fragment corresponding to the expected size was extracted from the gel using the GE Healthcare illustra GFX™ PCR DNA and Gel Band Purification Kit (Cat. No. 27-9602-01)

The plasmid pZMP3 1-Puro was digested with EcoRI prior to recombination in yeast with the gel extracted Herceptin scFv-Fc PCR fragment. One hundred .micro.l of competent yeast (S. cerevisiae) cells were combined with 25 .micro.l of the Herceptin scFv-Fc insert DNA and approximately 100 ng of EcoRI digested pZMP3 1-Puro vector, and the mix was transferred to a 0.2 cm electroporation cuvette. The yeast/DNA mixture was electropulsed using power supply (BioRad Laboratories, Hercules, Calif.) settings of 0.75 kV (5 kV/cm),infinity ohms, and 25 .micro.F. Six hundred .micro.l of 1.2 M sorbitol was added to the cuvette, and the yeast was plated in 300 .micro.l aliquots onto two URA-D plates and incubated at 30.deg.C. After about 72 hours, the Ura+yeast transformants from a single plate were resuspended in 1 ml H.sub.20 and spun briefly to pellet the yeast cells. The cell pellet was resuspended in 0.5 ml of lysis buffer (2% Triton X-100, 1% SDS, 100 mM NaCl, 10 mM Tris, pH 8.0, 1 mM EDTA). The five hundred .micro.l of the lysis mixture was added to an Eppendorf tube containing 250 .micro.l acid-washed glass beads and 300 .micro.l phenol-chloroform, was vortexed for 3 minutes, and spun for 5 minutes in an Eppendorf centrifuge at maximum speed. Three hundred .micro.l of the aqueous phase was transferred to a fresh tube, and the DNA was precipitated with 600 .micro.l ethanol, followed by centrifugation for 30 minutes at maximum speed. The tube was decanted and the pellet was washed with 1 mL of 70% ethanol. The tube was decanted and the DNA pellet was resuspended in 10 .micro.l water.

Transformation of electrocompetent E. coli host cells (DH10B) was done using 1 .micro.l of the yeast DNA preparation and 20 .micro.l of E. coli cells. The cells were electropulsed at 2.0 kV, 25 .micro.F, and 400 ohms. Following electroporation, 1 ml SOC (2% Bacto™ Tryptone (Difco, Detroit, Mich.), 0.5% yeast extract (Difco), 10 mM NaCl, 2.5 mM KC1, 10 mM MgCl.sub.2, 10 mM MgSO.sub.4, 20 mM glucose) was added and the cells were plated in 50 .micro.l and 200 .micro.l aliquots on two LB AMP plates (LB broth (Lennox), 1.8% Bacto.sup.™ Agar (Difco), 100 mg/L Ampicillin).

The inserts of six DNA clones for the construct were subjected to sequence analysis and one clone containing the correct sequence was selected. Large-scale plasmid DNA was isolated using a commercially available kit (QIAGEN Plasmid Mega Kit, Qiagen, Valencia, Calif.) according to manufacturer's instructions. The sequence of the insert DNA was the same as the cDNA sequence of the HER2/c-erb-2-binding scFv-scFc above.

2) Transfection and Expression in the pZMP3 1-Puro Vector:

The HER2/c-erb-2-binding scFv-Fc fusion construct in the pZMO31-Puro vector was produced transiently in 293F cells (Invitrogen, Carlsbad, Calif. Cat#R790-07). Briefly, 293F suspension cells were cultured in 293 Freestyle medium (Invitrogen, Carlsbad, Calif. Cat#12338-018) at 37.deg. C., 6% CO.sub.2 in three 3L spinners at 95 RPM. Fresh medium was added immediately prior to transfection to each of the spinners to obtain a 1.5 liter working volume at a final density of 1×10.sup.6 cells/ml. For each spinner, 2.0 mL of Lipofectamine 2000 (Invitrogen, Carlsbad, Calif. Cat#11668-019) was added to 20 mL Opti-MEM medium (Invitrogen, Carlsbad, Calif. Cat#31985-070) and 1.5 mg of construct DNA was diluted in a separate tube of 20 ml Opti-MEM. Each tube was incubated separately at room temperature for 5 minutes, then combined and incubated together for an additional 30 minutes at room temperature with occasional gentle mixing. The lipid-DNA mixture was then added to each spinner of 293F cells which were returned to 37.deg. C., 6% CO.sub.2 at 75 RPM. After approximately 96 hours, the conditioned medium was harvested and 0.2 .micro.M filtered. Protein expression was confirmed by Western blot, and the 293F cell pool was scaled-up for harvests for protein purification.

B) pZMP42 Expression Vector:

1) Construction of cDNA in Vector:

Plasmid pZMP42 is a mammalian expression vector containing an expression cassette having the chimeric CMV enhancer/MPSV promoter, an EcoRI site for linearization prior to yeast recombination, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

An expression construct containing a scFc comprising a HER2/c-erb-2-binding entity in an scFv-scFc configuration (previously described) with a 25 mer Gly-Ser linker linking the variable heavy and light chains, and a 5 mer Gly-Ser linker linking the scFv and scFc region was constructed via a three step-PCR and homologous recombination using a DNA fragment encoding the HER2/c-erb-2-binding scFv-scFc and the expression vector pZMP42. The CDNA sequence of the HER2/c-erb-2-binding scFv-scFc MCV23 is SEQ ID NO:47.

The PCR fragment encoding HER2/c-erb-2-binding scFv-scFc was constructed to contain a 5′ overlap with the pZMP42 vector sequence in the 5′ non-translated region, the HER2/c-erb-2-binding scFv coding region (nucleotides 58-813), the scFc coding sequence (nucleotides 829-2343), and a 3′ overlap with the pZMP42 vector in the poliovirus internal ribosome entry site region. The leader used was murine 26-10 VL signal sequence (nucleotides 1-57).The first PCR amplification reaction used the 5′ oligonucleotide “zc56738” (SEQ ID NO:49), the 3′ oligonucleotide “zc56624” (SEQ ID NO: 40). The second PCR amplification reaction used the 5′ oligonucleotide “zc56739” (SEQ ID NO: 50), and the 3′ oligonucleotide “zc56740” (SEQ ID NO: 51), and a previously generated DNA clone of the HER2/c-erb-2-binding scFv as the template with the cDNA sequence shown in SEQ ID NO:43. The encoded HER2/c-erb-2-binding scFv protein has the amino acid sequence shown in SEQ ID NO:44.

The third PCR amplification reaction used the 5′ oligonucleotide “zc56738” (SEQ ID NO: 49), and the 3′ oligonucleotide “zc56740” (SEQ ID NO: 51), and the first two previously generated PCR templates in an overlap PCR reaction.

The PCR amplification reaction conditions were as follows: 1 cycle, 95 .deg.C., 2 minutes; 30 cycles, 95 .deg.C., 15 seconds, followed by 55 .deg.C., 30 seconds, followed by 68 .deg.C., 1 minute 45 seconds. The PCR reaction mixture was run on a 1% agarose gel and the DNA fragment corresponding to the expected size was extracted from the gel using the GE Healthcare illustra GFXTM PCR DNA and Gel Band Purification Kit (Cat. No. 27-9602-01).

The plasmid pZMP42 (containing the scFc) was digested with EcoRI prior to recombination in yeast with the gel extracted Herceptin scFv PCR fragment. One hundred .micro.l of competent yeast (S. cerevisiae) cells were combined with 25 .micro.l of the Herceptin scFv insert DNA and approximately 100 ng of EcoRI digested pZMP42 vector, and the mix was transferred to a 0.2 cm electroporation cuvette. The yeast/DNA mixture was electropulsed using power supply (BioRad Laboratories, Hercules, Calif.) settings of 0.75 kV (5 kV/cm), infinity ohms, and 25 .micro.F. Six hundred .micro.l of 1.2 M sorbitol was added to the cuvette, and the yeast was plated in 300 .micro.l aliquots onto two URA-D plates and incubated at 30.deg.C. After about 72 hours, the Ura+yeast transformants from a single plate were resuspended in 1 ml H.sub.20 and spun briefly to pellet the yeast cells. The cell pellet was resuspended in 0.5 ml of lysis buffer (2% Triton X-100, 1% SDS, 100 mM NaCl, 10 mM Tris, pH 8.0, 1 mM EDTA). The five hundred .micro.l of the lysis mixture was added to an Eppendorf tube containing 250 .micro.l acid-washed glass beads and 300 .micro.l phenol-chloroform, was vortexed for 3 minutes, and spun for 5 minutes in an Eppendorf centrifuge at maximum speed. Three hundred .micro.l of the aqueous phase was transferred to a fresh tube, and the DNA was precipitated with 600 .micro.l ethanol, followed by centrifugation for 30 minutes at maximum speed. The tube was decanted and the pellet was washed with 1 mL of 70% ethanol. The tube was decanted and the DNA pellet was resuspended in 10 .micro.l water.

Transformation of electrocompetent E. coli host cells (DHIOB) was done using 1 .micro.l of the yeast DNA preparation and 20 .micro.l of E. coli cells. The cells were electropulsed at 2.0 kV, 25 .micro.F, and 400 ohms. Following electroporation, 1 ml SOC (2% Bacto.sup.™ Tryptone (Difco, Detroit, Mich.), 0.5% yeast extract (Difco), 10 mM NaCl, 2.5 mM KCl, 10 mM MgCl.sub.2, 10 mM MgSO.sub.4, 20 mM glucose) was added and the cells were plated in 50 .micro.l and 200 .micro.l aliquots on two LB AMP plates (LB broth (Lennox), 1.8% Bacto.sup.™ Agar (Difco), 100 mg/L Ampicillin).

The inserts of six DNA clones for the construct were subjected to sequence analysis and one clone containing the correct sequence was selected. Large-scale plasmid DNA was isolated using a commercially available kit (QIAGEN Plasmid Mega Kit, Qiagen, Valencia, Calif.) according to manufacturer's instructions. The sequence of the insert DNA is the same as the HER2/c-erb-2-binding scFv-scFc cDNA sequence described above (SEQ ID NOs:47 and 48).

2) Transfection and Expression in the pZMP42 Vector:

The HER2/c-erb-2-binding scFv-scFc was produced transiently in 293F cells (Invitrogen, Carlsbad, Calif. Cat#R790-07). Briefly, 293F suspension cells were cultured in 293 Freestyle medium (Invitrogen, Carlsbad, Calif. Cat#12338-018) at 37.deg. C., 6% CO.sub.2 in three 3 L spinners at 95 RPM.Fresh medium was added immediately prior to transfection to each of the spinners to obtain a 1.5 liter working volume at a final density of 1×10E6 cells/ml. For each spinner, 2.0 mL of Lipofectamine 2000 (Invitrogen, Carlsbad, Calif. Cat#11668-019) was added to 20 mL Opti-MEM medium (Invitrogen, Carlsbad, Calif. Cat#31985-070) and 1.5 mg of construct DNA was diluted in a separate tube of 20 ml Opti-MEM. Each tube was incubated separately at room temperature for 5 minutes, then combined and incubated together for an additional 30 minutes at room temperature with occasional gentle mixing. The lipid-DNA mixture was then added to each spinner of 293F cells which were returned to 37.deg. C., 6% CO.sub.2 at 75 RPM. After approximately 96 hours, the conditioned medium was harvested and 0.2 .micro.M filtered. Protein expression was confirmed by Western blot, and the 293F cell pool was scaled up for harvest and protein purification, as is described in example 4.

### EXAMPLE 9

### Stimulation of NK ADCC Activity Against SK-BR-3 with scFc Molecules

Method: Leukopheresed blood was obtained from an in-house donor program. Mononuclear cells (MNC) were prepared by ficoll centrifugation. Natural killer (NK) cells were purified from the MNC population by negative enrichment, utilizing a human NK cell negative enrichment kit (Stem Cell Technologies #14055, Vancouver, BC). Briefly, MNC were labeled with lineage specific antibodies (excluding the NK lineage) and were in turn magnetically labeled. The labeled MNC were then run over a magnetic column where the labeled cells were retained and the non-labeled NK cells flowed through.

NK cells were plated at a density of 1×10.sup.6/mL and cultured for 6 days in .alpha.MEM/10% FBS/50.micro.M .beta.mercaptoethanol (Invitrogen, Carlsbad, Calif.), in the presence or absence of 20 ng/mL hIL-21 (in-house produced) at 37.deg.C., 5% CO.sub.2. At the end of the culture period, NK cells were harvested, washed into Hanks Buffered Saline Solution (Invitrogen, Carlsbad, Calif.) containing 5% FBS (HBSSF), counted, and placed into an antibody dependent cellular cytotoxicity (ADCC) assay, utilizing the human breast cancer cell line SK-BR-3 (ATCC, Manassas, Va. cat no HTB-30), which overexpress the HER2/c-erb-2 gene product, as the cytolytic target. NK cells (effectors) were added to round bottom 96 well plates at a concentration of 50,000/well in the top row, then serially diluted 1:3 five times, leaving cells in a volume of 100.micro.l. SK-BR-3 cells were labeled prior to the assay by incubating 60 minutes at 37.deg.C. in HBSSF with 10 .micro.M calcein AM (Molecular Probes, cat no C1430). The targets took up the fluorescent dye (calcein AM) and cytoplasmically converted it into the active fluorochrome, which is only released from the cell upon lysis. Calcein-loaded SK-BR-3 cells were then washed, pelleted, and resuspended to a concentration of 50,000 cells/ml in HBSSF. Antibody was added to yield final concentrations of 20, 6.7, 2.2, 0.74, and 0.25 .micro.g/ml when 100 .micro.l (5000 cells) of SK-BR-3 were added to an equal volume of NK cells. Duplicate wells were plated at each effector:target ratio and antibody concentration. Additionally, targets were plated into sextuplicate wells of 0.2% Triton X-100 to yield a “total lysis” value, and sextuplicate wells of HBSSF to yield a “non-specific release” value. Plates were spun at 600 rpm for 2 minutes to bring effectors and targets together in the bottom of the wells, and incubated at 37.deg.C., 5%CO.sub.2 for 3 hrs. After the incubation, plates were spun for 8 minutes at 1000 rpm to pellet cells. Lysed cells released the fluorochrome into the supernatant, 100 .micro.l of which was then harvested, transferred to a new flat bottom 96 well plate, and the amount of fluorescence quantitated in a fluorometer. The % cell lysis was calculated from the amount of fluorescence present in the supernatant after the 3-hour incubation in the presence or absence of varying amounts of NK cells (effectors) using the following formula: % Lysis=((Average sample RFU-non specific release RFU)/(total lysis RFU-non specific release RFU))X100. For the ADCC assay, targets were used with 20, 6.7, 2.2, 0.74, or 0.25 .micro.g/ml of a test agent (1=scFc10.1 with HER2/c-erb-2-binding scFv; 2=scFc10 with HER2/c-erb-2-binding scFv; 3=control Fc10; 4=control scFc10.1; or 5=Herceptin (Trastuzumab) (Genentech, Palo Alto, Calif.).

Result: IL-21-stimulated NK cells lyse antibody coated targets via Fc.gamma.;RIII binding. In order to test whether the molecules of the invention are capable of mediating this activity, IL-21-stimulated NK cells were exposed to SK-BR-3 cells in the presence of the test agent in an ADCC assay. The two control proteins, Fc10 and scFc10.1, did not stimulate any detectable NK lytic activity against SK-BR-3 targets at any concentration tested at any Effector:Target (E:T) ratio. The scFc proteins with the HER2/c-erb-2-binding scFvs (e.g., scFc10.1 with HER2/c-erb-2-binding scFv (SEQ ID NO:48) and Fc10 with HER2/c-erb-2-binding scFv (SEQ ID NO:60)), as well as the Herceptin, stimulated NK lytic activity against SK-BR-3 targets up to 35-50% at the highest tested E:T of 10. This activity did not decrease until the antibody concentration was below 0.74 .micro.g/ml indicating that, at 0.74 .micro.g/ml and above, the antibody concentration was saturating. The lytic activity stimulated by Herceptin and the scFc10.1 with HER2/c-erb-2-binding scFv appeared virtually identical at these saturating antibody concentrations, with the activity stimulated by the scFc10 with HER2/c-erb-2-binding scFv 5-10% lower at every E:T tested.

### EXAMPLE 10

### Complement Dependent Lysis Activity HER2/c-erb-2-Binding scFv/scFc1 on SK-BR-3 and MCF7

### Breast Cancer Cell Lines

Materials/Methods: The SK-BR-3 and MCF7 breast cancer cell lines (Cat #HTB-30 and HTB-22, respectively, ATCC Manassas, Va.), were grown to 80% confluency, and then harvested using Versene (Invitrogen, Carlsbad, Calif.). Cells were washed with Assay Buffer, (Hanks Balanced Salt Solution containing 1% Bovine Serum Albumen (Invitrogen) counted, and then resuspended at a concentration of 1-2×10.sup.6 cells per mL in assay buffer. Calcein AM (Invitrogen) was then added to cells at a final concentration of 10.micro.M. Cells were mixed and then placed at 37.deg.C. for 1 hour for labeling. Following labeling, cells were then washed in assay buffer and resuspended at a concentration of 4×10.sup.5 cells per mL in assay buffer.

Freshly thawed aliquots of Herceptin (Genentech, South San Francisco, Calif.), HER2/c-erb-2-binding scFv/scFc10.1 SEQ ID NO:48), HER2/c-erb-2-binding scFv/Fc10 (SEQ ID NO:60), Human Fc10 (SEQ ID NO:10), Human scFc molecule (SEQ ID NO:4), were diluted to a concentration of 40 or 50 .micro.g/ml in assay buffer, and then plated and serially diluted in duplicate into a 96-well round bottom microtiter plate. Calcein-labeled SK-BR-3 or MCF7 cells were then added to all wells (2×10.sup.4 cells in 50.micro.L giving a final volume of 100.micro.L per well. Cells and test proteins were then incubated for 30 minutes at 4.deg.C. before addition of complement.

Freshly isolated human serum was used as the complement source. Briefly, 20 mL of whole human blood was collected into untreated glass tubes and kept on ice until processing. Blood was allowed to clot on ice and then was spun down at 3000 rpm for 20 minutes at 4.deg.C. Serum was then pipetted off and either kept at 4.deg.C. for less than 1 hour before using in the assay or stored at −80.deg.C. to preserve complement activity. Freshly isolated or thawed serum was then diluted to 10% in assay buffer and 100.micro.L was added to all wells. Control wells were also included containing complement alone (non-specific lysis), no complement, or 100.micro.L 1% Triton X-100 (for 100% lysis). Plates were tapped gently to mix and then incubated for 2 hours at 37.deg.C.

Following incubation, plates were spun down at 300×g for 5 minutes and 100.micro.L of supernatant from each well was transferred to a 96-well flat-bottom mitrotiter plate. Plates were then analyzed for Calcein AM release using a Victor Wallac fluorescent plate reader. Data was then transferred into Excel for analysis and percent specific lysis was calculated for each experimental sample.

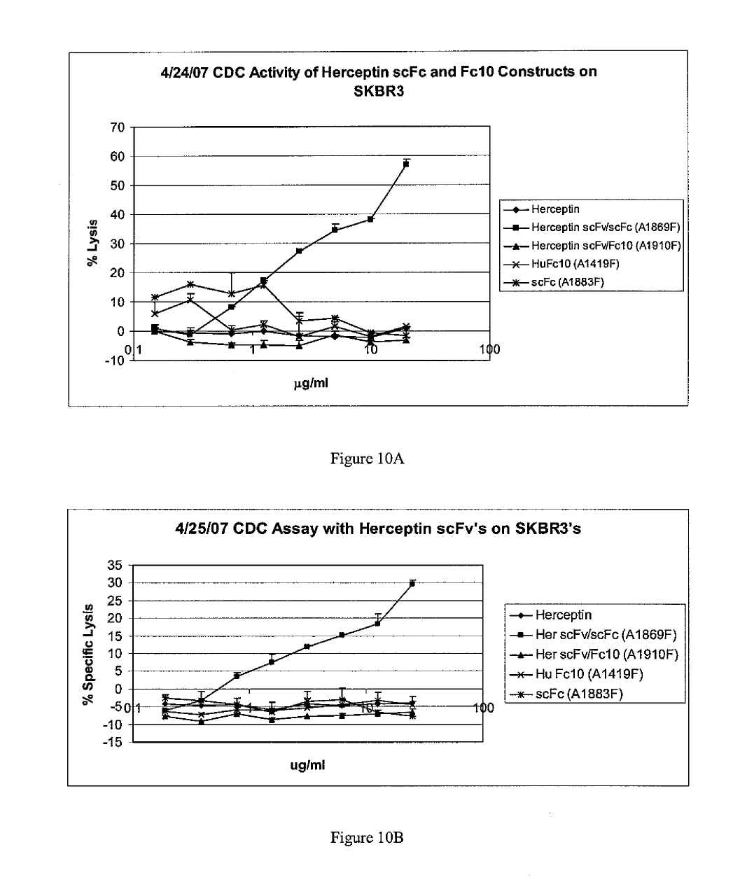

Results: HER2/c-erb-2-binding scFv/scFc1 was shown to mediate complement dependent lysis of SK-BR-3 breast cancer cells in a dose-dependent manner. In one experiment, using freshly isolated human serum as the complement source, the maximal lysis was at 20 .micro.g/ml was 57% (see FIG. 10A and Table 3). In a subsequent experiment, using freeze/thawed human serum as the complement source, maximal lysis at 25 .micro.g/ml was 30% (see FIG. 10B and Table 4). Both assays were set up in an identical manner, so the difference in maximal lysis was likely due to the complement source.

In contrast Herceptin, HER2/c-erb-2-binding scFv/Fc10 and the corresponding Fc control proteins were unable to mediate complement dependent lysis of SK-BR-3 breast cancer cells. The results with Herceptin and HER2/c-erb-2-binding scFv/Fc 10 are consistent with literature findings that suggest Herceptin is unable to mediate complement dependent lysis of breast cancer cell lines. (Prang, et al., Br J Cancer, 2005, Jan 31; 92(2):342-9) The enhanced CDC activity of the HER2/c-erb-2-binding scFv/scFc1 protein indicates that the structure of our HER2/c-erb-2-binding scFv/scFc1 may have a unique effector activity on breast cancer cell lines as compared to Herceptin.

In addition to SK-BR-3, the CDC activity of Herceptin, HER2/c-erb-2-binding scFv /scFc, and HER2/c-erb-2-binding scFv/Fc10 was also tested on a Her-2 low breast cancer cell line, MCF7. None of the test proteins showed CDC activity on this line, which is likely due to the low level of Her-2 antigen expressed on this cell line.

TABLE 3
CDC assay with SK-BR-3 targets and fresh complement.

| | | | Her- | Fc10 with | scFc10.1 with |
|---|---|---|---|---|---|
| Conc. | | | ceptin | HER2-binding | HER2-binding |
| (· micro · | Control | Control | anti- | scFv SEQ | scFv SEQ |
| g/mL) | (Fc10) | (scFc) | body | ID NO: 60 | ID NO: 48 |
| 20 | 1 ± 0 | −2 ± 1 | 1 ± 0 | −3 ± 1 | 57 ± 2 |
| 10 | −2 ± 1 | −1 ± 0 | −2 ± 0 | −4 ± 1 | 38 ± 0 |
| 5 | 2 ± 1 | 4 ± 0 | −2 ± 0 | −1 ± 1 | 34 ± 2 |
| 2.5 | −2 ± 1 | 3 ± 2 | −1 ± 8 | −5 ± 2 | 27 ± 0 |
| 1.25 | 2 ± 1 | 16 ± 2 | 0 ± 3 | −5 ± 1 | 17 ± 1 |
| 0.62 | 0 ± 2 | 13 ± 7 | −1 ± 1 | −5 ± 1 | 8 ± 0 |
| 0.31 | 10 ± 2 | 16 ± 0 | −1 ± 2 | −4 ± 1 | −1 ± 2 |
| 0.15 | 6 ± 4 | 12 ± 0 | 0 ± 2 | 0 ± 0 | 1 ± 0 |

TABLE 4
CDC assay with SK-BR-3 targets and thawed complement.

| | | | Her- | Fc10 with | scFc1 with |
|---|---|---|---|---|---|
| Conc. | | | ceptin | HER2-binding | HER2-binding |
| (· micro · | Control | Control | anti- | scFv SEQ | scFv SEQ |
| g/mL) | (Fc10) | (scFc1) | body | ID NO: 60 | ID NO: 48 |
| 25 | −5 ± 1 | −8 ± 0 | −4 ± 2 | −6 ± 1 | 30 ± 1 |
| 12.5 | −3 ± 2 | −7 ± 1 | −4 ± 1 | −7 ± 0 | 18 ± 3 |
| 6.2 | −5 ± 1 | −3 ± 3 | −5 ± 2 | −7 ± 0 | 15 ± 0 |
| 3.1 | −5 ± 1 | −3 ± 1 | −4 ± 3 | −8 ± 0 | 12 ± 0 |
| 1.6 | −6 ± 0 | −7 ± 1 | −6 ± 2 | −9 ± 1 | 8 ± 2 |
| 0.8 | −6 ± 1 | −5 ± 2 | −4 ± 0 | −7 ± 0 | 3 ± 1 |
| 0.4 | −7 ± 0 | −3 ± 0 | −5 ± 4 | −9 ± 0 | −3 ± 0 |
| 0.2 | −6 ± 0 | −3 ± 1 | −4 ± 2 | −8 ± 0 | −6 ± 1 |

### EXAMPLE 11

### Sialylated scFc Polypeptides

The glycosylation content of the described single chain Fc can be manipulated. A sialylated scFc will be obtained by expressing an scFc polypeptide in a production cells line such as CHO, NSO or other cell line transfected with alpha-2,3-sialyltransferase or alpha -2,6-sialyltransferase to either introduce a missing activity or enhance the endogenous levels of sialylation (See e.g., Ujita-Lee, et al., J. Biological Chemistry, 264:13845 (1989); Minch, et al., Biotechnol. Prog., 11:348 (1995)). Sialylation of polypeptides has been enhanced by modifying the growth conditions, for example, by adding 10 mM ManNac to the growth media. ManNac is a limiting precursor in the sialylation process (Bork, et al., FEBS letters 579:5079 (2005)). A production cell line could also be engineered to express a mutated GNE enzyme that leads to excessive sialylation due to lack of feed-back control (Bork, et al., FEBS letters 579:5079 (2005)). Sialylation of scFc could be further enhanced by introducing a point mutation (FA243) that facilitates sialylation (Lund, et al., J. Immunol., 157:4963 (1996)). A sialylated scFc could also be purified or enriched through affinity chromatography to a lectin that binds preferentially to alpha-2,6 sialic acid (Sambucus nigra, e.g., Shibuya, et al., Archives of Biochemistry and Biophysics, 254 (1): 1 (1987)). In order to optimize sialylation of an scFc polypeptide any of the processes described above could be used alone or in different combinations.

A non-fucosylated form of an scFc molecule can also be generated by expressing an scFc molecule in a cell line unable to add fucose. Alpha 1,6 fucosyltransferase and GDP-mannose 4,6-dehydratase are two of the enzymes known to play a role in adding fucose residues to sugar chains. In this example, fucosylation enzyme expression will be knocked-down by introducing shRNA expression vectors as has been done in CHO cells. (See e.g., Imai-Nishiya, et al., BMC Biotechnology 7:84 (2007)). An scFc molecule of the current invention will be expressed in these engineered CHO cell lines, and thus will lack fucose residues. In turn, expressed scFc molecules will have increased sialylation compared to a plurality of scFc molecules expressed in non-engineered cells.

The activity of a sialylated scFc molecule can be tested in a mouse model of anti-collagen Ab-induced arthritis with 5 to 10 mice per group. Sialylated and desialylalted scFc polypeptide preparations will be administered at 1 mg, 0.3 and 0.1 mg/mouse intravenously. Control mice will receive 20 mg/ml human IgG which is known to significantly reduce disease in this model. Approximately one hour after the administration of a sialylated scFc polypeptide or IgG the mice would receive anti-collagen Antibodies (Chondrex, Redmond, Wash.). Three days later mice will receive 50 .micro.l of LPS intaperitoneally. Paw thickness will be scored from the beginning of the experiment and registered daily for up to three weeks. The group treated with sialylated scFc will then be compared to the group treated with human IgG to determine efficacy of the sialylated molecules.

### EXAMPLE 12

### Stimulation of NK ADCC Activity Against MRC-5 Cells with scFc Molecules

Materials/Methods: Leukopheresed blood was obtained from an in-house donor program. Mononuclear cells (MNCs) were prepared by ficoll centrifugation. Natural killer (NK) cells were purified from the MNC population by negative enrichment, utilizing a human NK cell negative enrichment kit (Miltenyi Biotec, Auburn, Calif., #130-092-657). Briefly, MNCs were labeled with lineage specific antibodies (excluding the NK lineage) and were in turn magnetically labeled. The labeled MNCs were then run over a magnetic column where the labeled cells were retained and the non-labeled NK cells flowed through.

NK cells were plated at a density of 2×106/mL and cultured for 2 days in RPMI 1640/10% human AB serum or 10% FBS/2 mM GlutaMAX/1 mM sodium pyruvate/50 .micro.M beta mercaptoethanol (Invitrogen, Carlsbad, Calif.), in the presence of 10 ng/mL hIL-21 (SEQ ID NO:61) at 37.deg.C., 5% CO.sub.2. At the end of the culture period, NK cells were harvested, washed into Hanks Buffered Saline Solution (Invitrogen, Carlsbad, Calif.) containing 5% FBS (HBSSF), counted, and placed into an antibody dependent cellular cytotoxicity (ADCC) assay, utilizing the human lung fibroblast cell line MRC-5 (ATCC, Manassas, Va. #CCL-171), which express PDGFR.beta., as the cytolytic target. NK cells (effectors) were added to round-bottom 96 well plates at a concentration of 20,000/well in the top row, then serially diluted 1:3 five times, leaving cells in a volume of 100 .micro.L. MRC-5 cells were labeled prior to the assay by incubating 60 minutes at 37.deg.C., 5% CO.sub.2 in DMEM-F12 with 1× insulin/transferring/selenium (Invitrogen, Carlsbad, Calif.) with 2.5 .micro.M calcein AM (Invitrogen, Carlsbad, Calif., #C1430). The targets took up the fluorescent dye (calcein AM) and cytoplasmically converted it into the active fluorochrome, which is only released from the cell upon lysis. Calcein-loaded MRC-5 cells were then trypsinized, washed, pelleted, and resuspended to a concentration of 20,000 cells/mL in HBSSF. Test agents were added to yield final concentrations of 180, 60, and 15 nM when 100 .micro.L (2000 cells) of MRC-5 cells were added to an equal volume of NK cells. Duplicate wells were plated at each effector:target ratio and antibody concentration. Additionally, targets were plated into sextuplicate wells of 1% IGEPAL to yield a “total lysis” value, and sextuplicate wells of HBSSF to yield a “non-specific release” value. Plates were spun at 600 rpm for 2 minutes to bring effectors and targets together in the bottom of the wells, and incubated at 37.deg.C., 5% CO.sub.2 for 3 hrs. After the incubation, plates were spun for 8 minutes at 1000 rpm to pellet cells. Lysed cells released the fluorochrome into the supernatant, 100 .micro.L of which was then harvested, transferred to a new flat-bottom 96-well plate, and the amount of fluorescence quantitated using a Wallac fluorescent plate reader. The % cell lysis was calculated from the amount of fluorescence present in the supernatant after the 3-hour incubation in the presence or absence of varying amounts of NK cells (effectors) using the following formula: % Lysis=((Average sample RFU-non specific release RFU)/(total lysis RFU-non specific release RFU))×100. For the ADCC assay, targets were used with 60 nM of a test agent. For MRC-5 targets, 1=control (no test agent added); 2=anti-PDGFR.beta. monoclonal antibody; 3=Fc10 with PDGFR.beta.-binding scFv (SEQ ID NO:70); 4=scFc10.1 with PDGFR.beta.-binding scFv (SEQ ID NO:68).

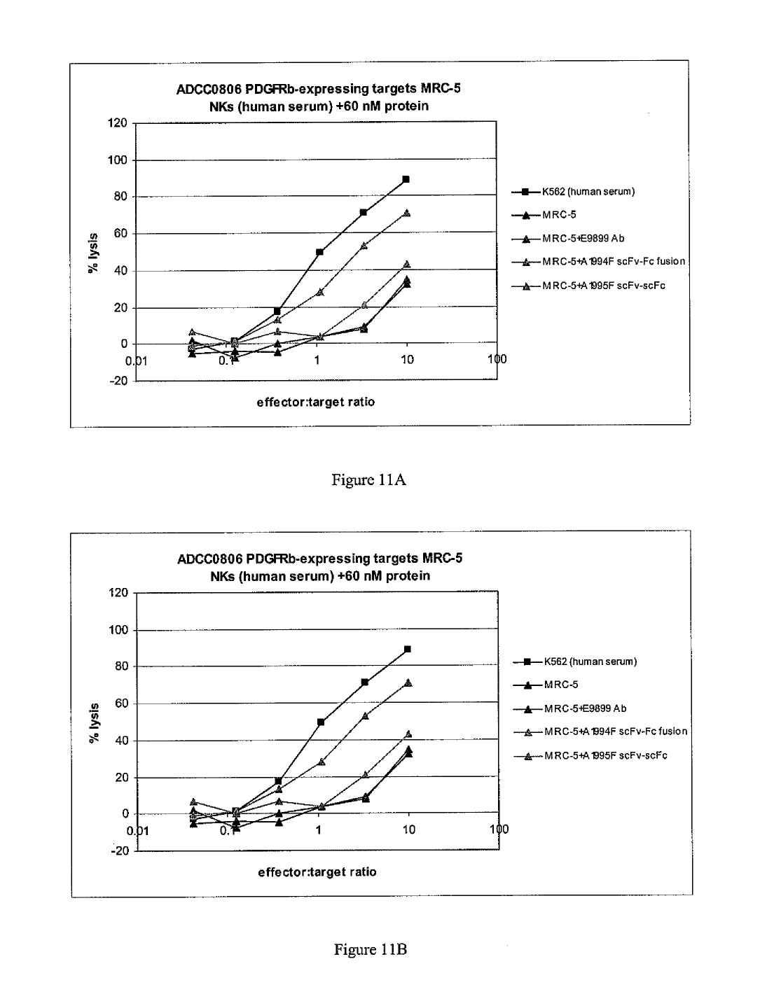

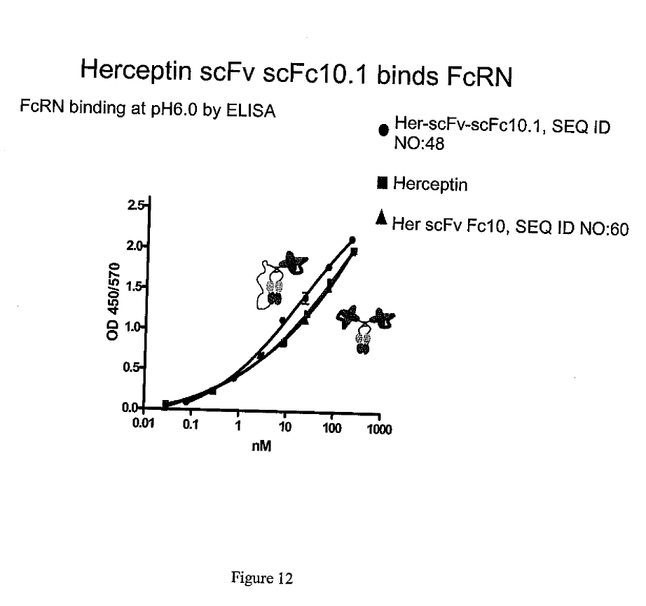

Results: As in Example 9, the molecules of the invention were tested in an ADCC assay to determine if they are capable of mediating ADCC activity. IL-2 1-stimulated NK cells were exposed to MRC-5 cells in the presence of the test agent in an ADCC assay. The results were different, depending on the source of serum used to stimulate the NK cells. For NKs grown in human serum, there was a small increase in cytolysis of targets comparing control (with no test agent; 60% killing at the highest E:T of 10) to the addition of anti-PDGFR.beta. antibody (70% killing at an E:T of 10). Table 5 and FIG. 11A. There was a greater increase in cytolysis when the NKs were grown in FBS (40% killing in the control compared to 60% killing with the anti-PDGFR.beta. antibody). Cytolysis by the scFc10.1 with PDGFR.beta.-binding scFv was ≧100% with both types of serum. However, the Fc10 with PDGFR.beta.-binding scFv showed an 80% cytolytic activity in human serum, but only 30% in FBS. Table 6 and FIG. 11B.

TABLE 5
ADCC with NKs cells grown in human serum,
MRC-5 targets, and 60 nM test agents

| | | | Fc10 with | scFc10.1 with |
|---|---|---|---|---|
| | | anti- | PDGFR.beta.- | PDGFR.beta.- |
| | | PDGFR.beta. | binding scFv | binding scFv |
| E:T | control | antibody | SEQ ID NO: 70 | SEQ ID NO: 68 |
| 10:1 | 61 ± 2 | 69 ± 12 | 80 ± 4 | 111 ± 15 |
| 3:1 | 33 ± 8 | 41 ± 3 | 33 ± 10 | 83 ± 14 |
| 1:1 | 2 ± 1 | 17 ± 5 | 21 ± 7 | 59 ± 1 |

TABLE 6
ADCC with NKs cells grown in FBS, MRC-5
targets, and 60 nM test agents.

| | | | Fc10 with | scFc10.1 with |
|---|---|---|---|---|
| | | anti- | PDGFR.beta.- | PDGFR.beta.- |
| | | PDGFR.beta. | binding scFv | binding scFv |
| E:T | control | antibody | SEQ ID NO: 70 | SEQ ID NO: 68 |
| 10:1 | 43 ± 3 | 62 ± 20 | 28 ± 4 | 102 ± 2 |
| 3:1 | 16 ± 5 | 24 ± 11 | 9 ± 0 | 59 ± 8 |
| 1:1 | 0 ± 4 | 8 ± 3 | 0 ± 4 | 37 ± 10 |

### EXAMPLE 13

### Immuno-Fluoresence Based Internalization Assay for Measuring the Effect of PDGFR.beta./VEGFA

### Antagonists on Receptor Internalization Over Time: A Comparison of an Fc10 with Anti-PDGFRbeta-Binding

### scFv Dimer, scFc10.1 with Anti-PDGFRbeta-Binding scFv Monomer and the Parent Murine Monoclonal PDGFRbeta Aantibody

Material and Methods:

Low passage Human Brain Vascular Pericytes (HBVP) (ScienCell Research, San Diego, Calif.) are plated at sub-confluency on 4 chamber glass Lab-TekII chamber slides (catalog #154917 Nalgene Nunc, Naperville, Ill.) at volume of 500 .micro.l/chamber in complete media (ScienCell Pericyte Media (PM) plus ScienCell supplements Fctal Bovine Serum, Pericyte Growth Supplement, and Penicillin-Streptomycin). Chamber slides are incubated at 37.deg.C. and 5% CO.sub.2 for 1-2 days until they reach approximately 75% confluency. The binding and internalization profiles of three PDGFR.beta./VEGFA antagonist antibodies are compared at time 0, 30 minutes, 60 minutes, 120 minutes and 180 minutes. Initial binding is done at 4.deg.C. (T0), so all slides are placed on ice and washed one time with cold DMEM +0.1% BSA. The PDGFR.beta./VEGFA antagonists are then diluted to 1.micro.g/ml in binding buffer consisting of DMEM+3% BSA and Hepes buffer. Each slide is configured so that three antagonists and one well with no treatment is designated for each chamber slide. A separate slide is set up with secondary antibody only controls. Five-hundred .micro.l/well of antagonists or media only is added to each chamber slide. Following a one hour incubation, the T0 slide is fixed by washing with cold PBS one time and adding 1 ml/well paraformaldehyde solution. This T0 slide measures receptor expression on the cell surface and the slides incubated at 37.deg.C. measure receptor internalization over time. The remaining slides are put in the 37.deg.C. incubator and removed and fixed in a similar fashion at thirty minutes, sixty minutes, 2 hour and three hour time points. All slides are kept on ice after fixation. Once all of the slides have been fixed, they are washed one time with PBS and permeabilized for two minutes with −20.deg.C. MetOH. The slides are washed again with cold PBS. From now on the staining is done at room temperature. The slides are incubated at room temperature for five minutes in 50 mM Glycine made up in PBS. The glycine is removed and washed off with PBS, and the slides are blocked in 10% normal goat serum in PBS (#S-1000, Vector Labs, Inc. Burlingame, Calif.), 500.micro.1/well for thirty minutes. Following the blocking step, 500.micro.1/well of the secondary antibodies is added to every well. Alexafluor 488 goat anti-mouse (Cat. #A11029, Molecular Probes, Eugene, Oreg.) is added to the wells containing the parent PDGFR.beta. monoclonal antibody. Alexafluor 488 goat anti-human (Cat. #A11013, Molecular Probes, Eugene, Oreg.) is added to the wells containing Fc10 with anti-PDGFRbeta-binding scFv (SEQ ID NO:70) or containing scFc10.1 with anti-PDGFRbeta-binding scFv (SEQ ID NO:68). Both secondary antibodies are diluted 1:150 in wash buffer consisting of PBS +0.1% Tween 20 and 0.1% BSA. The slides are incubated in the dark at room temperature for forty-five minutes. Each slide is washed three times by soaking in PBS for 5 minutes at room temperature. One drop of Vectashield mounting medium with DAPI stain is added to each chamber (Cat. #H-1200, Vector Labs, Inc., Burlingame Calif.) and then the slides are coverslipped and examined under the fluorescent microscope. Metavue software is used to visualize the two-color staining profile.

Results: Cell surface plasma membrane staining is apparent with all three molecules at the T0 time point. A clear pattern of internalization is apparent as early as 30 minutes post 37.deg.C. incubation with the dimer Fc10 with anti-PDGFRbeta-binding scFv (SEQ ID NO:70) and the PDGFR.beta. monoclonal antibody parental antibody. The scFc10.1 molecule with an anti-PDGFRbeta-binding scFv (SEQ ID NO:68), on the other hand, is very poorly internalized in human brain vascular pericytes.

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

What is claimed is:

1. An scFc polypeptide comprising at least two Fc monomers and at least one linker.

2. The scFc polypeptide of claim 1, wherein said scFc polypeptide comprises a first Fc monomer comprising a CH2 domain and a CH3 domain and a second Fc monomer comprising a CH2 domain and a CH3 domain.

3. The scFc polypeptide of claim 2, wherein said first Fc monomer and said second Fc monomer are arranged in an amino to carboxyl order selected from:
a) Hinge-CH2-CH3-linker-Hinge-CH2-CH3;
b) Hinge-CH2-CH3-linker-CH2-CH3;
c) Hinge-CH2-linker-Hinge-CH2-CH3-linker-CH3;
d) Hinge-CH2-linker-CH2-CH3-linker-CH3;
e) linker-CH2-CH3-linker-CH2-CH3; and
f) CH2-linker-CH2-CH3-linker-CH3.

4. The scFc polypeptide of claim 1, wherein said linker is selected from the group consisting of: SEQ ID NO:52, SEQ ID NO:53, SEQ ID NO:54, SEQ ID NO:55, SEQ ID NO:56, SEQ ID NO:11 and SEQ ID NO:33.

5. The scFc polypeptide of claim 1, wherein said scFc polypeptide further comprises one or more binding entities.

6. The scFc polypeptide of claim 5, wherein said binding entity is selected from:
a) a scFv;
b) a Fab;
c) a diabody;
d) a triabody;
e) a single-domain antibody;
f) a recombinant antibody fragment;
g) a tascFv; and
h) a biscFv.

7. The scFc polypeptide of claim 5, wherein said binding entity is a soluble receptor or a ligand-binding fragment thereof.

8. The scFc polypeptide of claim 5, wherein said one or more binding entities are connected to the scFc by one or more polypeptide linkers.

9. The scFc polypeptide of claim 5, wherein said scFc polypeptide comprises two Fc monomers and the amino to carboxyl order of said Fc monomers and said one or more binding entities is selected from:
a) Fc monomer-binding entity-Fc monomer-binding entity;
b) binding entity-Fc monomer-Fc monomer-binding entity;
c) binding entity-binding entity-Fc monomer-Fc monomer;
d) Fc monomer-Fc monomer-binding entity-binding entity;
e) Fc monomer-binding entity-Fc monomer;
b) binding entity-Fc monomer-Fc monomer; and
c) Fc monomer-Fc monomer-binding entity.

10. The scFc polypeptide of claim 1, wherein said scFc polypeptide further comprises at least one functional molecule selected from: a) a therapeutic agent, b) a molecule that increases solubility of said scFc polypeptide compared to an scFc polypeptide without said molecule, c) a molecule that improves stability of said scFc polypeptide compared to an scFc polypeptide without said molecule, d) a molecule that extends the half life of said scFc polypeptide compared to an scFc polypeptide without said molecule, a sialylic acid and a combination thereof.

11. The scFc polypeptide of claim 10, wherein said molecule that extends the half life of the scFc polypeptide is PEG.

12. The scFc polypeptide of claim 1, wherein each of said at least two Fc monomers have a polypeptide sequence that is at least 90% identical to an Fc monomer polypeptide sequence of an Fc molecule selected from the group consisting of: SEQ ID NO:4; SEQ ID NO:6; SEQ ID NO:8; SEQ ID NO:10; SEQ ID NO:22; SEQ ID NO:31, SEQ ID NO:57 and SEQ ID NO:58.

13. The scFc polypeptide of claim 1, wherein said scFc polypeptide comprises two Fc monomers, a linker and further comprises at least one binding entity.

14. The scFc polypeptide of claim 13, wherein said two Fc monomers have a polypeptide sequence that is at least 90% identical to the Fc monomers of SEQ ID NO:4, SEQ ID NO:22 or SEQ ID NO: 31.

15. The scFc polypeptide of claim 14, wherein said scFc polypeptide comprises one binding entity.

16. The scFc polypeptide of claim 15, wherein said binding entity binds a PDGFR.beta. antigen.

17. The scFc polypeptide of claim 15, wherein said scFc polypeptide is SEQ ID NO:68.

18. The scFc polypeptide of claim 13, wherein said two Fc monomers have a polypeptide sequence that is at least 90% identical to the Fc monomers of SEQ ID NO: 4, SEQ ID NO:22 or SEQ ID NO: 31.

19. The scFc polypeptide of claim 18, wherein said scFc polypeptide comprises one binding entity.

20. The scFc polypeptide of claim 19, wherein said binding entity binds a HER2/c-erb-2 antigen.

21. The scFc polypeptide of claim 19, wherein said scFc polypeptide is selected from the group: SEQ ID NO:48.

22. The scFc polypeptide of claim 14, wherein said scFc polypeptide comprises two binding entities.

23. The scFc polypeptide of claim 22, wherein said scFc polypeptide is tascFv-scFc; BiscFv-scFc; or bispecific-scFc.

24. The scFc polypeptide of claim 23, wherein said two binding entities each bind a separate antigen.

25. The scFc polypeptide of claim 24, wherein said separate antigens are present on the same cell and binding said separate antigens increases avidity relative to when only one of said separate antigens is present on a cell.

26. The scFc polypeptide of claim 24, wherein a first binding entity binds a PDGFR.beta. antigen and a second binding entity binds a VEGF-A antigen.

27. The scFc polypeptide of claim 1, wherein said scFc polypeptide sequence is at least 95% identical to SEQ ID NO:4, and wherein said binding entity is at least 90% identical to a polypeptide sequence selected from the group consisting of SEQ ID NO:44 and SEQ ID NO:38.

28. The scFc polypeptide of claim 27, wherein said scFc polypeptide sequence is SEQ ID NO:4.

29. The scFc polypeptide of claim 28, wherein said binding entity is at least 95% identical to a polypeptide sequence selected from the group consisting of SEQ ID NO:44 and SEQ ID NO:38.

30. The scFc polypeptide of claim 27, wherein said binding entity is SEQ ID NO:38.

31. The scFc polypeptide of claim 27, wherein said scFc polypeptide is SEQ ID NO:48.

32. A polynucleotide molecule comprising a polynucleotide sequence encoding the scFc polypeptide of claim 1.

33. The polynucleotide molecule of claim 32, wherein said polynucleotide molecule is an expression vector further comprising the following operably linked elements:
(a) a transcription promoter; and
(b) a transcription terminator.

34. The polynucleotide molecule of claim 33, wherein said polynucleotide encodes a polypeptide with at least 90% sequence identity to a polypeptide selected from the group consisting of: SEQ ID NO:4; SEQ ID NO:6; SEQ ID NO:8; SEQ ID NO:10; SEQ ID NO:22; SEQ ID NO:31; SEQ ID NO:57 and SEQ ID NO:58.

35. The polynucleotide molecule of claim 33, wherein said polynucleotide encodes a polypeptide with at least 95% sequence identity to SEQ ID NO:4.

36. The polynucleotide molecule of claim 34, wherein said encoded polypeptide is SEQ ID NO:4.

37. The polynucleotide molecule of claim 33, further comprising a polynucleotide sequence encoding at least one binding entity.

38. The polynucleotide molecule of claim 37, wherein a binding entity binds an antigen selected from the group consisting of: PDGFR.beta., and HER2/c-erb-2.

39. The polynucleotide molecule of claim 38, wherein said polynucleotide encodes a polypeptide that is 95% identical to SEQ ID NO:48.

40. The polynucleotide molecule of claim 38, wherein said polynucleotide encodes a polypeptide that is SEQ ID NO:48.

41. The polynucleotide molecule of claim 38, wherein said polynucleotide encodes a polypeptide that is 95% identical to SEQ ID NO:68.

42. The polynucleotide molecule of claim 38, wherein said polynucleotide encodes a polypeptide that is SEQ ID NO:68.

43. A cultured cell comprising the scFc polypeptide expression vector according claim 33.

44. The cultured cell of claim 43, wherein said cell further expresses a sialyltransferase gene.

45. The cultured cell of claim 44, wherein said cell is a yeast cell.

46. The cultured cell of claim 44, wherein said cell is a mammalian cell.

47. The cultured cell of claim 44, wherein said cell is a Chinese Hamster Ovary cell engineered to express an alpha-2,6-sialyltransferase gene.

48. The cultured cell of claim 47, wherein said scFc polypeptide expression vector comprises a polynucleotide sequence that is 90% identical to SEQ ID NO:47.

49. The cultured cell of claim 48, wherein said scFc polypeptide expression vector comprises a polynucleotide sequence that is SEQ ID NO:47.

50. The cultured cell of claim 47, wherein said scFc polypeptide expression vector comprises a polynucleotide sequence that is 90% identical to SEQ ID NO:67.

51. The cultured cell of claim 50, wherein said scFc polypeptide expression vector comprises a polynucleotide sequence that is SEQ ID NO:67.

52. A method of producing an scFc polypeptide comprising:
culturing a cell according to claim 43 under conditions wherein an scFc polynucleotide is expressed from said scFc polypeptide expression vector; and
recovering said expressed scFc polypeptide.

53. The method of claim 52, wherein said cell expresses a sialyltransferase gene.

54. The method of claim 53, wherein said cell is a Chinese Hamster Ovary cell engineered to express an alpha-2,6-sialyltransferase gene.

55. An isolated polypeptide comprising a polypeptide sequence that is 90% identical to SEQ ID NO: 48.

56. The isolated polypeptide of claim 55, wherein said polypeptide sequence is SEQ ID NO: 48.

57. The isolated polypeptide of claim 55, wherein said polypeptide further comprises a sialylic acid sugar residue.

58. An isolated polypeptide comprising a polypeptide sequence that is 90% identical to SEQ ID NO: 68.

59. The isolated polypeptide of claim 58, wherein said polypeptide sequence is SEQ ID NO:68.

60. The isolated polypeptide of claim 58, wherein said polypeptide further comprises a sialylic acid sugar residue.

61. A method for treating an immune system disorder in a mammal suspected of suffering from such a disorder comprising administering to said mammal an scFc polypeptide of claim 1.

62. The method of claim 61, wherein said scFc polypeptide comprises two Fc monomers, and wherein said two Fc monomers have polypeptide sequences with at least 90% identity to the respective Fc monomer sequences in the group consisting of: SEQ ID NO:4; SEQ ID NO:6; SEQ ID NO:8; SEQ ID NO:10; SEQ ID NO:22; SEQ ID NO:31; SEQ ID NO:57; and SEQ ID NO:58.

63. The method of claim 61, wherein said scFc polypeptide further comprises at least one binding entity that entity binds an antigen selected from the group consisting of: IL-17A and IL-23.

64. A method for treating a cancer in a mammal suspected of suffering from such a disorder comprising administering to said mammal an scFc polypeptide of claim 1.

65. The method of claim 64, wherein said scFc polypeptide comprises two Fc monomers, and wherein said two Fc monomers have polypeptide sequences with at least 90% identity to the respective Fc monomer sequences in the group consisting of: SEQ ID NO:4; SEQ ID NO:6; SEQ ID NO:8; SEQ ID NO:10; SEQ ID NO:22; SEQ ID NO:31; SEQ ID NO:57; and SEQ ID NO:58.

66. The method of claim 64, wherein said scFc polypeptide further comprises at least one binding entity.

67. The method of claim 66, wherein a binding entity binds an antigen selected from the group consisting of: PDGFR.beta., VEGF-A, and HER2/c-erb-2.

68. The method of claim 67, wherein said scFc polypeptide is at least 95% identical to SEQ ID NO:48.

69. The method of claim 68, wherein said scFc polypeptide is SEQ ID NO:48.

70. The method of claim 67, wherein said scFc polypeptide is at least 95% identical to SEQ ID NO:68.

71. The method of claim 70, wherein said scFc polypeptide is SEQ ID NO:68.

72. A method of stimulating NK cells in a mammal comprising admixing an scFc polypeptide of claim 1 with cells or tissues of said mammal.

73. The method of claim 72, wherein said scFc polypeptide further comprises a binding entity and is monovalent.

74. A method of stimulating CDC in a mammal comprising admixing an scFc polypeptide of claim 1 with cells or tissues of said mammal.

75. The method of claim 74, wherein said scFc polypeptide further comprises a binding entity and is monovalent.

76. A method of stimulating ADCC in a mammal comprising admixing an scFc polypeptide of claim 1 with cells or tissues of said mammal.

77. The method of claim 76, wherein said scFc polypeptide further comprises a binding entity and is monovalent.

78. The method of claim 77, wherein said scFc polypeptide stimulates an enhanced ADCC response relative to a monoclonal antibody targeting the same antigen.

79. The method of claim 78, wherein said antigen is HER2/c-erb-2.


SEQUENCE LISTING (72 sequences)

SEQ ID NO: 1 - DNA, 974 bp, Homo sapiens
atggatgcaatgaagagagggctctgctgtgtgctgctgctgtgtggcgccgtcttcgtttcgctcagccaggaaatccatgccgagttgagacgcttccgtagatctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaccgggtaaataaagcccgggcaatcttaaactagaggcgcgccgccagccccctgatggggg

SEQ ID NO: 2 - protein, 307 aa, Homo sapiens
MDAMKRGLCCVLLLCGAVFVSLSQEIHAELRRFRRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGPGK

SEQ ID NO: 3 - DNA, 1623 bp, Homo sapiens
atggatgcaatgaagagagggctctgctgtgtgctgctgctgtgtggcgccgtcttcgtttcgctcagccaggaaatccatgccgagttgagacgcttccgtagatctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaggcgggggaagcggggagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - protein, 541 aa, Homo sapiens
MDAMKRGLCCVLLLCGAVFVSLSQEIHAELRRFRRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 696 bp, Homo sapiens
gagcccagatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 232 aa, Homo sapiens
EPRSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 696 bp, Homo sapiens
gagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 232 aa, Homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 696 bp, Homo sapiens
gagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 41 aa, Artificial Sequence
  artificial
GGGGSGGGGSGGGGSGGGGSGGGGSGGGGSGGGGSGGGGSG

SEQ ID NO: 12 - DNA, 70 bp, Artificial Sequence
  oligonucleotide primer member
cagccaggaaatccatgccgagttgagacgcttccgtagatctgagcccaaatcttcagacaaaactcac

SEQ ID NO: 13 - DNA, 63 bp, Artificial Sequence
  oligonucleotide primer member
gccaccgcctccacttccgccaccgccggatcccccaccacctttacccggagacagggagag

SEQ ID NO: 14 - DNA, 57 bp, Artificial Sequence
  Oligonucleotide
ggaggcagcggcggaggtgggtctgggggtggaggctccggaccgggtaaataaagc

SEQ ID NO: 15 - DNA, 71 bp, Artificial Sequence
  Oligonucleotide
cccccatcagggggctggcggcgcgcctctagtttaagattgcccgggctttatttacccggtccggagcc

SEQ ID NO: 16 - DNA, 60 bp, Artificial Sequence
  Oligonucleotide
ggaagtggaggcggtggctctggtggtggcggatctggcggaggaggcagcggcggaggt

SEQ ID NO: 17 - DNA, 48 bp, Artificial Sequence
  oligonucleotide primer member
ggaggctccggaggcgggggaagcggggagcccaaatcttcagacaaa

SEQ ID NO: 18 - DNA, 40 bp, Artificial Sequence
  oligonucleotide primer member
gtttaagattgcccgggctttatttacccggagacaggga

SEQ ID NO: 19 - DNA, 924 bp, Homo sapiens
atggatgcaatgaagagagggctctgctgtgtgctgctgctgtgtggcgccgtcttcgtttcgctcagccaggaaatccatgccgagttgagacgcttccgtagatctgagcccaaatcttcagacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaccgggtaaataa

SEQ ID NO: 20 - protein, 307 aa, Homo sapiens
MDAMKRGLCCVLLLCGAVFVSLSQEIHAELRRFRRSEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGPGK

SEQ ID NO: 21 - DNA, 1578 bp, Homo sapiens
atggatgcaatgaagagagggctctgctgtgtgctgctgctgtgtggcgccgtcttcgtttcgctcagccaggaaatccatgccgagttgagacgcttccgtagatctgagcccaaatcttcagacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaggcgggggaagcggggcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 526 aa, Homo sapiens [sequence omitted]

SEQ ID NO: 23 - DNA, 70 bp, Artificial Sequence; identical to SEQ ID NO: 12
  oligonucleotide primer member

SEQ ID NO: 24 - DNA, 52 bp, Artificial Sequence
  oligonucleotide primer member
tcttcagacaaaactcacacatccccaccgtccccagcacctgaactcctgg

SEQ ID NO: 25 - DNA, 63 bp, Artificial Sequence; identical to SEQ ID NO: 13
  oligonucleotide primer member.

SEQ ID NO: 26 - DNA, 48 bp, Artificial Sequence; identical to SEQ ID NO: 17
  oligonucleotide primer member

SEQ ID NO: 27 - DNA, 40 bp, Artificial Sequence; identical to SEQ ID NO: 18
  oligonucleotide primer member

SEQ ID NO: 28 - DNA, 927 bp, Homo sapiens
atggatgcaatgaagagagggctctgctgtgtgctgctgctgtgtggcgccgtcttcgtttcgctcagccaggaaatccatgccgagttgagacgcttccgtagatctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaacctactaccactcccgcacctaggcctcctactcctgcaccaactattgcttcacaacctctaagtttaagacctgaagctagtcgacctgcagcaggtggctccggaccgggtaaataa

SEQ ID NO: 29 - protein, 308 aa, Homo sapiens
MDAMKRGLCCVLLLCGAVFVSLSQEIHAELRRFRRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKPTTTPAPRPPTPAPTIASQPLSLRPEASRPAAGGSGPGK

SEQ ID NO: 30 - DNA, 1626 bp, Homo sapiens
atggatgcaatgaagagagggctctgctgtgtgctgctgctgtgtggcgccgtcttcgtttcgctcagccaggaaatccatgccgagttgagacgcttccgtagatctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaacctactaccactcccgcacctaggcctcctactcctgcaccaactattgcttcacaacctctaagtttaagacctgaagctagtcgacctgcagcaggtggctccggaggcgggggaagcggggagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 31 - protein, 542 aa, Homo sapiens
MDAMKRGLCCVLLLCGAVFVSLSQEIHAELRRFRRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKPTTTPAPRPPTPAPTIASQPLSLRPEASRPAAGGSGGGGSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 102 bp, Homo sapiens
aaacctactaccactcccgcacctaggcctcctactcctgcaccaactattgcttcacaacctctaagtttaagacctgaagctagtcgacctgcagcaggt

SEQ ID NO: 33 - protein, 34 aa, Homo sapiens
KPTTTPAPRPPTPAPTIASQPLSLRPEASRPAAG

SEQ ID NO: 34 - DNA, 63 bp, Artificial Sequence
  oligonucleotide primer member
tgcaggagtaggaggcctaggtgcgggagtggtagtaggttttttacccggagacagggagag

SEQ ID NO: 35 - DNA, 62 bp, Artificial Sequence
  oligonucleotide
ctaagtttaagacctgaagctagtcgacctgcagcaggtggctccggaccgggtaaataaag

SEQ ID NO: 36 - DNA, 63 bp, Artificial Sequence
  oligonucleotide
cctaggcctcctactcctgcaccaactattgcttcacaacctctaagtttaagacctgaagct

SEQ ID NO: 37 - DNA, 1527 bp, Homo sapiens
atgaagttgcctgttaggctgttggtgctgatgttctggattcctgcttccagcagtgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggtggtggttctggcggcggcggctccggtggaggtggttctggtggtggtggttctggtggtggtggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgggtggaggtggttctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 38 - protein, 508 aa, Homo sapiens
MKLPVRLLVLMFWIPASSSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 70 bp, Artificial Sequence
  oligonucleotide primer member
actttgcctttctctccacaggtgtccagggaattcatataggccggccaccatgaagttgcctgttagg

SEQ ID NO: 40 - DNA, 57 bp, Artificial Sequence
  oligonucleotide primer member
actgctggaagcaggaatccagaacatcagcaccaacagcctaacaggcaacttcat

SEQ ID NO: 41 - DNA, 36 bp, Artificial Sequence
  oligonucleotide primer member
attcctgcttccagcagtgatatccagatgacccag

SEQ ID NO: 42 - DNA, 42 bp, Artificial Sequence
  oligonucleotide primer member
agaaccacctccacccgaggagacggtgaccagggttccttg

SEQ ID NO: 43 - DNA, 909 bp, Homo sapiens
atgaaaaagaatatcgcatttcttcttgcatctatgttcgttttttctattgctacaaacgcgtacgctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggtggtggttctggcggcggcggctccggtggaggtggttctggtggtggtggttctggtggtggtggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgcgggctgatgcttcggccgctggatccgaacaaaagctgatctcagaagaagacctaaactcacatcaccatcaccatcactaa

SEQ ID NO: 44 - protein, 302 aa, Homo sapiens
MKKNIAFLLASMFVFSIATNAYADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSRADASAAGSEQKLISEEDLNSHHHHHH

SEQ ID NO: 45 - DNA, 47 bp, Artificial Sequence
  oligonucleotide primer member
ggtggaggtggttctgagcccaaatcttcagacaaaactcacacatg

SEQ ID NO: 46 - DNA, 49 bp, Artificial Sequence
  oligonucleotide primer member
cccagagctgttttaaggcgcgcctctagatttatttacccggagacag

SEQ ID NO: 47 - DNA, 2346 bp, Homo sapiens
atgaagttgcctgttaggctgttggtgctgatgttctggattcctgcttccagcagtgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggtggtggttctggcggcggcggctccggtggaggtggttctggtggtggtggttctggtggtggtggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgggtggaggtggttctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaggcgggggaagcggggagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 48 - protein, 781 aa, Homo sapiens
MKLPVRLLVLMFWIPASSSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 71 bp, Artificial Sequence
  oligonucleotide primer member
actttgcctttctctccacaggtgtcctcgagaattcatataggccggccaccatgaagttgcctgttagg

SEQ ID NO: 50 - DNA, 51 bp, Artificial Sequence
  oligonucleotide primer member
attcctgcttccagcagtgatatccagatgacccagtccccgagctccctg

SEQ ID NO: 51 - DNA, 63 bp, Artificial Sequence
  oligonucleotide primer member
tgtgtgagttttgtctgaagatttgggctcagaaccacctccacccgaggagacggtgaccag

SEQ ID NO: 52 - protein, 15 aa, Artificial Sequence
  linker sequence
GGGGSGGGGSGGGGS

SEQ ID NO: 53 - protein, 17 aa, Artificial Sequence
  linker
GXXXXXGXXXXXGXXXX

SEQ ID NO: 54 - protein, 11 aa, Artificial Sequence
  Linker
GGGXGGGXGGG

SEQ ID NO: 55 - protein, 25 aa, Artificial Sequence
  linker
GXXXXXXXXXXXCGXXXXXXXXXXX

SEQ ID NO: 56 - protein, 11 aa, Artificial Sequence
  linker
GGGGGCGGGGG

SEQ ID NO: 57 - protein, 231 aa, Homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 223 aa, Homo sapiens
TCPPCPAPELLGGPSVFLFPPKPKDTLMISRTPEVTCVVVDVSHEDPEVKFNWYVDGVEVHNAKTKPREEQYESTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 1497 bp, Homo sapiens
atgggatggagctggatctttctctttcttctgtcaggaactgcaggtgtcctctctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgggcggcggcggctccggcgggggtggaagtggtggtggtggttctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggaggtggttctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 60 - protein, 498 aa, Homo sapiens
MGWSWIFLFLLSGTAGVLSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 134 aa, Homo sapiens
MQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 62 - DNA, 596 bp, Homo sapiens
ttctgaaatgagctgttgacaattaatcatcggctcgtataatgtgtggaattgtgagcggataacaatttcacacagaattcattaaagaggagaaattaactatgcaaggtcaagatcgccacatgattagaatgcgtcaacttatagatattgttgatcagctgaaaaattatgtgaatgacctggttccggaattcctgccggctccggaagatgttgagaccaactgtgagtggtccgctttctcctgtttccagaaagcccagctgaaatccgcaaacaccggtaacaacgaacgtatcatcaacgtttccattaaaaaactgaaacgtaaaccgccgtccaccaacgcaggtcgtcgtcagaaacaccgtctgacctgcccgtcctgtgattcttatgagaaaaaaccgccgaaagaattcctggaacgtttcaaatccctgctgcagaaaatgattcaccagcacctgtcctctcgtacccacggttccgaagattcctgatgttttggcggatgagataagattttcagcctgatacagattaaatcagaacgcagaagcggtctgataaaacagaatttgcctggc

SEQ ID NO: 63 - DNA, 2301 bp, Homo sapiens
atgaagttgcctgttaggctgttggtgctgatgttctggattcctgcttccagcagtgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggtggtggttctggcggcggcggctccggtggaggtggttctggtggtggtggttctggtggtggtggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgggtggaggtggttctgagcccaaatcttcagacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaggcgggggaagcggggcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 64 - protein, 766 aa, Homo sapiens
MKLPVRLLVLMFWIPASSSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 2349 bp, Homo sapiens
atgaagttgcctgttaggctgttggtgctgatgttctggattcctgcttccagcagtgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggtggtggttctggcggcggcggctccggtggaggtggttctggtggtggtggttctggtggtggtggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgggtggaggtggttctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaacctactaccactcccgcacctaggcctcctactcctgcaccaactattgcttcacaacctctaagtttaagacctgaagctagtcgacctgcagcaggtggctccggaggcgggggaagcggggagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 66 - protein, 782 aa, Homo sapiens [sequence omitted]

SEQ ID NO: 67 - DNA, 2334 bp, Homo sapiens
atgaactttgtgctcagcttgattttccttgccctcattttaaaaggtgtccagtgtgaagtgcagctggtggagtctgggggaggcttagtgaagcctggagggtccctgaaactctcctgtgcagcctctggattcgctttcagtagctatgccatgtcttgggttcgccagagtccggaaaagaggctggagtgggtcgcaaccattagcagtggtggtcattacaccttctatccagacagtgtgaagggtcgcttcaccatctccagagacaatgccaagaacaccctgtacctgcaaatgagcagtctgaggtctgaggacacggccatttattactgtgcaagacgttactatgctctggactactggggtcaaggaacctcagtcaccgtctcctcaggtggtggtggttctggcggcggcggctccggcgggggtggaagtggtggtggtggttctggtggtggtggttctgacattgtgatgacccagtctcaaaaattcatgtccacatcactaggagacagggtcagcgtctcctgcaaggccagtcagaatgtgcttactaatgtagcctggtatcaacaaaaaccagggcaatctcctaaaactctgatttattcggcatcctaccggtacagtggagtccctgatcgcttcacaggcagtggatctgggacagatttcactctcaccatcagcattgttcagtctgaagacttggcagagtatttctgtcaacaatataacatctatccgtggacgttcggtggaggcaccaagctggaaatcaaaggtggaggtggttctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggtgggggatccggcggtggcggaagtggaggcggtggctctggtggtggcggatctggcggaggaggcagcggcggaggtgggtctgggggtggaggctccggaggcgggggaagcggggagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 68 - protein, 777 aa, Homo sapiens
MNFVLSLIFLALILKGVQCEVQLVESGGGLVKPGGSLKLSCAASGFAFSSYAMSWVRQSPEKRLEWVATISSGGHYTFYPDSVKGRFTISRDNAKNTLYLQMSSLRSEDTAIYYCARRYYALDYWGQGTSVTVSSGGGGSGGGGSGGGGSGGGGSGGGGSDIVMTQSQKFMSTSLGDRVSVSCKASQNVLTNVAWYQQKPGQSPKTLIYSASYRYSGVPDRFTGSGSGTDFTLTISIVQSEDLAEYFCQQYNIYPWTFGGGTKLEIKGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 1515 bp, Homo sapiens
atgaactttgtgctcagcttgattttccttgccctcattttaaaaggtgtccagtgtgaagtgcagctggtggagtctgggggaggcttagtgaagcctggagggtccctgaaactctcctgtgcagcctctggattcgctttcagtagctatgccatgtcttgggttcgccagagtccggaaaagaggctggagtgggtcgcaaccattagcagtggtggtcattacaccttctatccagacagtgtgaagggtcgcttcaccatctccagagacaatgccaagaacaccctgtacctgcaaatgagcagtctgaggtctgaggacacggccatttattactgtgcaagacgttactatgctctggactactggggtcaaggaacctcagtcaccgtctcctcaggtggtggtggttctggcggcggcggctccggcgggggtggaagtggtggtggtggttctggtggtggtggttctgacattgtgatgacccagtctcaaaaattcatgtccacatcactaggagacagggtcagcgtctcctgcaaggccagtcagaatgtgcttactaatgtagcctggtatcaacaaaaaccagggcaatctcctaaaactctgatttattcggcatcctaccggtacagtggagtccctgatcgcttcacaggcagtggatctgggacagatttcactctcaccatcagcattgttcagtctgaagacttggcagagtatttctgtcaacaatataacatctatccgtggacgttcggtggaggcaccaagctggaaatcaaaggtggaggtggttctgagcccaaatcttcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 70 - protein, 504 aa, Homo sapiens
MNFVLSLIFLALILKGVQCEVQLVESGGGLVKPGGSLKLSCAASGFAFSSYAMSWVRQSPEKRLEWVATISSGGHYTFYPDSVKGRFTISRDNAKNTLYLQMSSLRSEDTAIYYCARRYYALDYWGQGTSVTVSSGGGGSGGGGSGGGGSGGGGSGGGGSDIVMTQSQKFMSTSLGDRVSVSCKASQNVLTNVAWYQQKPGQSPKTLIYSASYRYSGVPDRFTGSGSGTDFTLTISIVQSEDLAEYFCQQYNIYPWTFGGGTKLEIKGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1485 bp, Homo sapiens
atgaactttgtgctcagcttgattttccttgccctcattttaaaaggtgtccagtgtgaagtgcagctggtggagtctgggggaggcttagtgaagcctggagggtccctgaaactctcctgtgcagcctctggattcgctttcagtagctatgccatgtcttgggttcgccagagtccggaaaagaggctggagtgggtcgcaaccattagcagtggtggtcattacaccttctatccagacagtgtgaagggtcgcttcaccatctccagagacaatgccaagaacaccctgtacctgcaaatgagcagtctgaggtctgaggacacggccatttattactgtgcaagacgttactatgctctggactactggggtcaaggaacctcagtcaccgtctcctcaggcggcggcggctccggcgggggtggaagtggtggtggtggttctgacattgtgatgacccagtctcaaaaattcatgtccacatcactaggagacagggtcagcgtctcctgcaaggccagtcagaatgtgcttactaatgtagcctggtatcaacaaaaaccagggcaatctcctaaaactctgatttattcggcatcctaccggtacagtggagtccctgatcgcttcacaggcagtggatctgggacagatttcactctcaccatcagcattgttcagtctgaagacttggcagagtatttctgtcaacaatataacatctatccgtggacgttcggtggaggcaccaagctggaaatcaaaggtggaggtggttctgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 72 - protein, 494 aa, Homo sapiens
MNFVLSLIFLALILKGVQCEVQLVESGGGLVKPGGSLKLSCAASGFAFSSYAMSWVRQSPEKRLEWVATISSGGHYTFYPDSVKGRFTISRDNAKNTLYLQMSSLRSEDTAIYYCARRYYALDYWGQGTSVTVSSGGGGSGGGGSGGGGSDIVMTQSQKFMSTSLGDRVSVSCKASQNVLTNVAWYQQKPGQSPKTLIYSASYRYSGVPDRFTGSGSGTDFTLTISIVQSEDLAEYFCQQYNIYPWTFGGGTKLEIKGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK